(19) United States
(12) Patent Application Publication
(10) Pub. No.: US 2023/0398124 A1
(43) Pub. Date: Dec. 14, 2023

(54) SEVEN-MEMBERED RING COMPOUNDS AND METHOD OF USE THEREOF

(71) Applicant: Invea Therapeutics, Inc., Guilford, CT (US)

(72) Inventors: Krishnan Nandabalan, Guilford, CT (US); Sameer Sharma, Palampur (IN)

(21) Appl. No.: 18/208,241

(22) Filed: Jun. 9, 2023

Related U.S. Application Data
(60) Provisional application No. 63/429,004, filed on Nov. 30, 2022.
(60) Provisional application No. 63/350,961, filed on Jun. 10, 2022.

(51) Int. Cl. A61K 31/551; A61P 1/16

(52) U.S. Cl. CPC A61K 31/551; A61P 1/16

(57) ABSTRACT

The present disclosure relates to uses of compounds of formula (I) in treating or preventing variety of diseases, or conditions in a subject particularly hepatobiliary disease, gastrointestinal diseases associated with hepatobiliary disease and/or eosinophilic diseases using therapeutic effective amounts of compound of general formula (I):

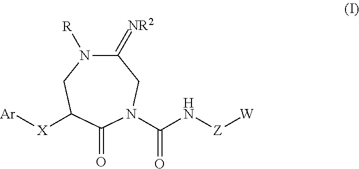

or a pharmaceutically acceptable salt or solvate thereof, wherein the variables R¹, R², Ar, X, Z and W have the meaning as described herein.

### CROSS REFERENCE

This application claims priority to U.S. Provisional Application No. 63/350,961 filed Jun. 10, 2022 and U.S. Provisional Application No. 63/429,004 filed Nov. 30, 2022, the contents of each of which are herein incorporated by reference in their entirety.

### FIELD

The present disclosure provides 7-membered nitrogen-containing ring compound having a chymase inhibitory activity and uses thereof in treating hepatobiliary disease.

### BACKGROUND

Hepatobiliary diseases include a heterogeneous group of diseases of the liver and biliary system. They result from the impairment in bile formation or flow. Clinical consequence may include jaundice, pruritis, and fatigue. If left untreated may lead to fibrosis, biliary cirrhosis and end stage liver disease. Hepatobiliary diseases and hepatobiliary-related diseases include acute hepatitis, alcoholic hepatitis, alcoholic liver disease, extrahepatic cholestasis, colitis hepatic fibrosis, liver abscess, liver cirrhosis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, nonalcoholic steatohepatitis, polycystic liver disease, primary biliary cirrhosis, veno-occlusive disease, Wilson's disease. The two most common Hepatobiliary diseases are primary sclerosing cholangitis (PSC) and Primary biliary cholangitis (PBC).

PSC is estimated to affect approximately 53,000 people in the United States and approximately 126,000 people in 7MM countries. The market size for PSC was estimated to be $46 million in the United States and $126 million globally in 2020 and is expected to grow to approximately $157 million in the United States and approximately $157 million by 2026. PSC is characterized by a progressive course of cholestasis with inflammation and fibrosis of the intrahepatic and extrahepatic bile ducts. Symptoms associated with PSC include fatigue, pruritus, jaundice, dark urine, light-colored stools, abdominal pain, and/or nausea, hepatomegaly, and cirrhosis. Mast cells are known to play an active role in the pathogenesis of PSC. Chymase positive mast cells have been detected in portal areas and sinusoidal walls, in sections from diseased livers, coinciding with zones of fibrosis which in turn relate to extensive hepatic fibrosis. The 80% of patients with PSC have underlying inflammatory bowel disease (IBD) and shows an increased risk of colorectal cancer compared to non PSC-IBD.

PBC is characterized with inflammation and scarring of the small bile ducts. In severe cases PBC can lead to jaundice, cirrhosis and eventually liver failure. PBC is divided into four stages from stage 1 being early disease, without significant scarring of the liver and stage 4 being cirrhosis. The exact prevalence of PBC is estimated to be approximately 131,000 people in the United States and approximately 304,000 people globally.

Colitis is a chronic digestive disease characterized by inflammation of the inner lining of the colon. Infection, loss of blood supply in the colon, Inflammatory Bowel Disease (IBD) and invasion of the colon wall with collagen or lymphocytic white blood cells are all possible causes of an inflamed colon. Common tests for colitis include X-rays of the colon, testing the stool for blood and pus, sigmoidoscopy and colonoscopy. Additional tests include stool cultures and blood tests, including blood chemistry tests. A high erythrocyte sedimentation rate (ESR)—a measure of how long it takes for red blood cells to settle in a blood sample—is typical of acute colitis. Colitis encompasses both Ulcerative Colitis (UC) and Crohn's disease (CD). Ulcerative colitis (UC), an inflammatory bowel disease (IBD), is characterized by chronic inflammation of the colon and rectum. It is a lifelong condition of the digestive tract with a high unmet medical need. The pathogenesis of IBD is multifactorial and includes genetic predisposition, epithelial barrier defects, dysregulated immune responses, and environmental factors.

While there are many treatments and therapies for these conditions, none are ideal. The current therapies can provide only supportive and symptomatic improvement. For example, OCALIVA, Obeticholic acid, a semisynthetic bile acid analogue chemical structure: 6α-ethyl-chenodeoxycholic acid is approved drug for treating chronic hepatobiliary disease but has black box warning. Liver transplantation is the only proven long-term treatment of hepatobiliary disease.

Thus, there is a high unmet need in hepatobiliary disease for the development of an economical and orally available pharmacotherapy that exerts a disease modifying effect.

### SUMMARY

The present disclosure encompasses a method for treating or preventing hepatobiliary disease in a subject in need thereof, said method comprising administering to said subject a therapeutic effective amount of a compound of Formula (I), or a pharmaceutically acceptable salt or solvate thereof,

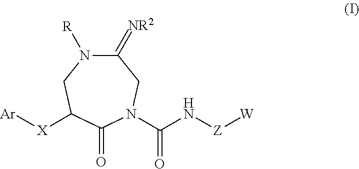

wherein,
Ar is (1) a C6 to C14 aromatic hydrocarbon group, (2) a 5 to 8-membered aromatic heterocyclic group having 1 to 4 hetero atoms selected from the group consisting of a nitrogen atom, sulfur atom, and oxygen atom, in place of a carbon atom, or (3) a bicyclic or tricyclic aromatic group formed by condensation of the above aromatic heterocyclic group and a C6 to C14 aromatic hydrocarbon ring,
where the above groups (1) to (3) of Ar are optionally substituted with 1 to 5 groups selected from the group consisting of:
(i) a halogen atom,
(ii) nitro,
(iii) cyano,
(iv) C1 to C6 alkyl optionally substituted with 1 to 3 halogen atoms,
(v) C2 to C6 alkenyl optionally substituted with 1 to 3 halogen atoms,
(vi) C2 to C6 alkynyl optionally substituted with 1 to 3 halogen atoms,
(vii) C3 to C6 cycloalkyl,
(viii) hydroxyl,
(ix) C1 to C6 alkoxy optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(x) C1 to C5 alkylenedioxy,
(xi) C1 to C6 alkylthio optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(xii) amino,
(xiii) mono-C1 to C6 alkylamino,
(xiv) di-C1 to C6 alkylamino,
(xv) 5- to 6-membered cyclic amino,
(xvi) C1 to C6 alkylcarbonyl,
(xvii)carboxyl,
(xviii) C1 to C6 alkoxycarbonyl,
(xix) carbamoyl,
(xx) thiocarbamoyl,
(xxi) mono-C1 to C6 alkylcarbamoyl,
(xxii) di-C1 to C6 alkylcarbamoyl,
(xxiii) 5- to 6-membered cyclic aminocarbonyl,
(xxiv) sulfo,
(xxv) C1 to C6 alkylsulfonyl,
(xxvi) C1 to C6 alkoxycarbonylamino,
(xxvii) C1 to C6 alkylcarbonylamino,
(xxviii) mono- or di-C1 to C6 alkylaminocarbonylamino,
(xxix) aminosulfonyl, and
(xxx) mono- or di-C1 to C6 alkylaminosulfonyl, or substituted with 1 to 9 deuterium atoms,
X is (1) a connecting bond, (2) linear or branched C1 to C6 alkylene optionally substituted with 1 to 12 deuterium atoms, (3) an oxygen atom, (4) NR³, where R³ is a hydrogen atom or a C1 to C6 alkyl group, or (5) —S(O)m-, where m is an integer of 0 to 2,
Z is (1) a connecting bond or (2) CR⁴R⁵ where R⁴ and R⁵ are, independently,
(A) a hydrogen atom,
(B) a deuterium atom,
(C) C1 to C6 alkyl optionally substituted with 1 to 5 groups selected from the group consisting of (i) carboxyl, (ii) C1 to C6 alkoxycarbonyl, (iii) phenyl, (iv) hydroxyl, (v) C1 to C6 alkoxy, (vi) halogen atom, and (vii) C3 to C6 cycloalkyl, or substituted with 1 to 13 deuterium atoms,
(D) C3 to C6 cycloalkyl optionally substituted with 1 to 5 groups selected from the group consisting of (i) a halogen atom and (ii) an alkyl group optionally substituted with 1 to 3 halogen atoms or optionally substituted, with 1 to 11 deuterium atoms,
(E) COOR⁶, where R⁶ is a hydrogen atom or C1 to C6 alkyl, or
(F) CONR⁷R⁸, where R⁷ and R⁸ are, independently,
(a) hydrogen atom,
(b) C1 to C6 alkyl optionally substituted with 1 to 3 groups selected from the group consisting of (i) a halogen atom, (ii) C3 to C6 cycloalkyl, (iii) carboxyl, (iv) C1 to C6 alkoxycarbonyl, (v) C1 to C6 alkylcarbonyl, (vi) carbamoyl, (vii) mono-C1 to C6 alkylcarbamoyl, (viii) di-C1 to C6 alkylcarbamoyl, (ix) C6 to C12 aryl, and (x) C1 to C10 heteroaryl,
(c) C6 to C14 aromatic hydrocarbon group,
(d) a 5- to 8-membered aromatic heterocyclic group having 1 to 4 hetero atoms selected from the group consisting of a nitrogen atom, sulfur atom, and oxygen atom, in place of a carbon atom, or
(e) a bicyclic or tricyclic aromatic group formed by condensation of the above aromatic heterocyclic, group and a C6 to C14 aromatic hydrocarbon ring,
where each of the groups (c) to (e) is optionally substituted with 1 to 5 groups selected from the group consisting of (i) a halogen atom, (ii) nitro, (iii) cyano, (iv) C1 to C6 alkyl optionally substituted with 1 to 3 halogen atoms, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 halogen atoms, (x) C1 to C5 alkylenedioxy, (xi) C1 to C6 alkylthio optionally substituted with 1 to 3 halogen atoms, (xii) amino, (xiii) mono-C1 to C6 alkylamino, (xiv) di-C1 to C6 alkylamino, (xv) 5- to 6-membered cyclic amino, (xvi) C1 to C6 alkylcarbonyl, (xvii) carboxyl, (xviii) C1 to C6 alkoxycarbonyl, (xix) carbamoyl, (xx) thiocarbamoyl, (xxi) mono-C1 to C6 alkylcarbamoyl, (xxii) di-C1 to C6 alkylcarbamoyl, (xxiii) C6 to C10 arylcarbamoyl, (xxiv) C1 to C10 heteroarylcarbamoyl, (xxv) sulfo, (xxvi) C1 to C6 alkylsulfonyl, (xxvii) aminosulfonyl, and (xxviii) mono- or di-C1 to C6 alkylaminosulfonylamino, or substituted with 1 to 9 deuterium atoms,
W is (1) a hydrogen atom, (2) a C6 to C14 aromatic hydrocarbon group, (3) a 5- to 8-membered aromatic heterocyclic group having 1 to 4 hetero atoms selected from a nitrogen atom, a sulfur atom, and an oxygen atom, in place of a carbon atom, (4) a bicyclic, or tricyclic aromatic group formed by condensation of the above aromatic heterocyclic group and a C6 to C14 aromatic hydrocarbon ring, or a deuterium atom,
where, each of the groups (2) to (4) of the above W is optionally substituted with 1 to 5 groups selected from the group consisting of (i) a halogen atom, (ii) nitro, (iii) cyano, (iv) C1 to C6 alkyl optionally substituted with 1 to 3 groups selected from the group consisting of a halogen atom, amino, C1 to C6 alkoxycarbonyl, C1 to C6 alkoxycarbonylamino, and carboxyl, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 groups selected from the group consisting of a halogen atom, hydroxyl, C1 to C6 alkoxy, amino and mono- or di-C1 to C6 alkylamino, (x) C1 to C5 alkylenedioxy, (xi) C1 to C6 alkylthio optionally substituted with 1 to 3 groups selected from the group consisting of a halogen atom, hydroxyl, C1 to C6 alkoxy, amino, and mono- or di-C1 to C6 alkylamino, (xii) amino, (xiii) mono-C1 to C6 alkylamino, (xiv) di-C1 to C6 alkylamino, (xv) 5- to 6-membered cyclic amino, (xvi) C1 to C6 alkyl-carbonyl, (xvii) carboxyl, (xviii) C1 to C6 alkoxycarbonyl optionally substituted with a halogen atom, (xix) C7 to C16 aralkyloxycarbonyl optionally substituted with a halogen atom, (xx) carbamoyl, (xxi) mono-C1 to C6 alkylcarbamoyl optionally substituted with 1 to 3 groups which are selected from the group consisting of a halogen atom, hydroxyl, carboxyl, C1 to C6 alkoxy, amino, and mono- or di-C1 to C6 alkylamino, (xxii) di-C1 to C6 alkylcarbamoyl optionally substituted with hydroxyl, (xxiii) 5- to 6-membered cyclic aminocarbonyl optionally substituted with C1 to C6 alkoxycarbonyl, (xxiv) C6 to C10 arylcarbamoyl, (xxv) C1 to C10 heteroarylcarbamoyl, (xxvi) C7 to C16 aralkylcarbamoyl, (xxvii) C1 to C10 heteroaryl-C1 to C6 alkylcarbamoyl, (xxviii) N C1 to C6 alkyl-N—C6 to C12 arylcarbamoyl, (xxix) C3 to C6 cycloalkylcarbamoyl, (xxx) sulfo, (xxxi) C1 to C6 alkylsulfonyl, (xxxii) C1 to C6 alkylsulfonylamino, (xxxiii) C6 to C12 arylsulfonylamino optionally substituted with C1 to C6 alkyl, (xxxiv) C1 to C10 heteroarylsulfonylamino, (xxxv) C1 to C6 alkoxycarbonylamino, (xxxvi) C1 to C6 alkylcarbonylamino, (xxxvii) mono- or di-C1 to C6 alkylaminocarbonylamino, (xxxviii) C6 to C12 aryl, (xxxix) C1 to C10 heteroaryl, (xl) C6 to C10 aryloxy, (xli) C1 to C10 heteroaryloxy, (xlii) C7 to C16 aralkyloxy, (xliii) C1 to C10 heteroaryl-C1 to C6 alkyloxy, (xliv) aminosulfonyl, (xlv) mono- or di-C1 to C6 alkylaminosulfonyl, (xlvi) C7 to C16 aralkyloxycarbamoyl, and (xlvii) C1 to C10 heteroaryl-C1 to C6 alkyloxycarbamoyl, or substituted with 1 to 9 deuterium atoms,
R1 is
(1) a hydrogen atom,
(2) C1 to C6 alkyl optionally substituted with 1 to 3 of (i) a halogen atom or (ii) C3 to C6 cycloalkyl or substituted with (iii) 1 to 13 deuterium atoms,
(3) C2 to C6 alkenyl optionally substituted with 1 to 3 of (i) a halogen atom or (ii) C3 to C6 cycloalkyl or substituted with (iii) 1 to 11 deuterium atoms,
(4) C2 to C6 alkynyl optionally substituted with 1 to 3 of (i) a halogen atom or (ii) C3 to C6 cycloalkyl, or substituted with (iii) 1 to 9 deuterium atoms, or
(5) C3 to C6 cycloalkyl optionally substituted with 1 to 3 of (i) a halogen atom or (ii) C3 to C6 cycloalkyl, or substituted with (iii) 1 to 11 deuterium atoms,
R2 is (1) OR⁹ or (2) NR¹⁰R¹¹
where, R⁹, R¹⁰, and R¹¹ independently indicate
(A) a hydrogen atom,
(B) C1 to C6 alkyl,
(C) C2 to C6 alkenyl,
(D) C3 to C6 alkynyl,
(E) C3 to C6 cycloalkyl
(F) C6 to C14 aromatic hydrocarbon group,
(G) a 5- to 8-membered aromatic heterocyclic group having 1 to 4 hetero atoms selected from the group consisting of a nitrogen atom, sulfur atom, and oxygen atom, in place of a carbon atom, or
(H) a bicyclic or tricyclic aromatic group formed by condensation of the above aromatic heterocyclic group and a C6 to C14 aromatic hydrocarbon ring,
where each of the groups of the above (B) to (E) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5 to 6-membered cyclic amino, (ix) carboxyl, (x) C1 to C6 alkoxycarbonyl, (xi) C1 to C6 alkylcarbonyl, (xii) carbamoyl, (xiii) mono-C1 to C6 alkylcarbamoyl, (xiv) di-C1 to C6 alkylcarbamoyl, (xv) C6 to C12 aryl, and (xvi) C1 to C10 heteroaryl, or substituted with 1 to 13 deuterium atoms,
further, each of the groups of the above (F) to (H) are optionally substituted with 1 to 5 groups which are selected from the group consisting of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms, or R¹ and R² may form 5- or 6-member heterocyclic ring together with the atoms they are bonded with, where the above 5- or 6-member heterocyclic ring is optionally substituted with 1 to 3 groups selected from the group consisting of
(A) a halogen atom,
(B) oxo,
(C) hydroxyl
(D) C1 to C6 alkyl,
(E) C2 to C6 alkenyl,
(F) C2 to C6 alkynyl,
(G) C3 to C6 cycloalkyl
(H) C6 to C14 aromatic hydrocarbon group,
(I) 5- to 8-membered aromatic heterocyclic group having 1 to 4 hetero atoms selected from the group consisting of a nitrogen atom, sulfur atom, and oxygen atom, in place of a carbon atom, and
(J) a bicyclic or tricyclic aromatic group formed by condensation of the above aromatic heterocyclic group and a C6 to C14 aromatic hydrocarbon ring, or substituted with
(K) 1 to 6 deuterium atoms,
where each of the groups of the above (D) to (G) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5- to 6-membered cyclic amino, (ix) carboxyl, (x) C1 to C6 alkoxycarbonyl, (xi) C1 to C6 alkyl-carbonyl, (xii) carbamoyl, (xiii) mono-C1 to C6 alkylcarbamoyl, (xiv) di-C1 to C6 alkylcarbamoyl, (xv) C6 to C12 aryl and (xvi) C1 to C10 heteroaryl, or substituted with 1 to 13 deuterium atoms, each of the groups of the above (H) to (J) is optionally substituted with 1 to 5 groups which are selected from the group consisting, of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms.

In some embodiments, the compound of Formula (I) or a pharmaceutically acceptable salt or solvate thereof, is selected from the group consisting of:
(1) 4-{(1R)-1-[({(6R)-6-(5-chloro-2-methoxybenzyl)-7-oxo-3-[(pyridin-2-yloxy)imino]-1,4-diazepan-1-yl}carbonyl)amino]propyl}benzoic acid,
(2) 2-amino-4-[(1R)-1-({[(6S)-6-(5-chloro-2-methoxybenzyl)-3-(ethoxyimino)-7-oxo-1,4-diazepan-1-yl]carbonyl}amino)butyl]benzoic acid,
(3) 2-amino-4-{(1R)-1-[({(6S)-6-(5-chloro-2-methoxybenzyl)-7-oxo-3-[(2,2,2-trifluoroethoxy)imino]-1,4-diazepan-1-yl}carbonyl)amino]butyl}benzoic acid,
(4) 2-amino-4-{(1R)-1-[({(6R)-6-(5-chloro-2-methoxybenzyl)-3-[(3,5-difluorophenoxy)imino]-7-oxo-1,4-diazepan-1-yl}carbonyl)amino]ethyl}benzoic acid, (5) 2-amino-4-[(1R)-1-({[(6S)-6-(5-chloro-2-methoxybenzyl)-3-(methoxyimino)-4-methyl-7-oxo-1,4-diazepan-1-yl]carbonyl}amino)butyl]benzoic acid,
(6) (4-{(1R)-1-[({(6R)-6-(5-chloro-2-methoxybenzyl)-3-[(3,5-difluorophenoxy)imino]-7-oxo-1,4-diazepan-1-yl}carbonyl)amino]ethyl}phenyl) acetic acid,
(7) 2-amino-4-{[({(6R)-6-(5-chloro-2-methoxybenzyl)-3-[(3,5-difluorophenoxy)imino]-7-oxo-1,4-diazepan-1-yl}carbonyl)amino]methyl}benzoic acid,
(8) {4-[(1R)-1-({[(6R)-6-(5-chloro-2-methoxybenzyl)-7-oxo-3-(phenoxyimino)-1,4-diazepan-1-yl]carbonyl}amino)propyl]phenyl}acetic acid,
(9) 2-amino-4-[(1R)-1-({[(6R)-6-(5-chloro-2-methoxybenzyl)-7-oxo-3-(phenoxyimino)-1,4-diazepan-1-yl]carbonyl}amino)propyl]benzoic acid, or
(10) 2-amino-4-[(1R)-1({[(6R)-6-(5-chloro-2-methoxybenzyl)-3-(ethoxyimino)-7-oxo-1,4-diazepan-1-yl]carbonyl}amino)butyl]benzoic acid.

In some embodiments, the compound of Formula (I) or a pharmaceutically acceptable salt or solvate thereof is 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid (herein referred as “compound 1”).

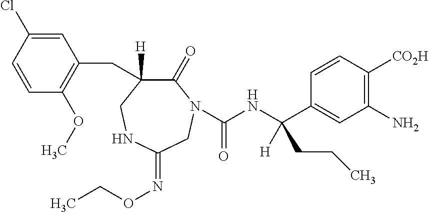 [SMILES: CCC[C@@H](NC(=O)N1C/C(=N/OCC)NC[C@H](Cc2cc(Cl)ccc2OC)C1=O)c1ccc(C(=O)O)c(N)c1]

In a preferred embodiment, the compound 1 is a monoacetic acid solvate known as 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid monoacetic acid solvate (herein referred as “Compound 1 monoacetic acid solvate”)

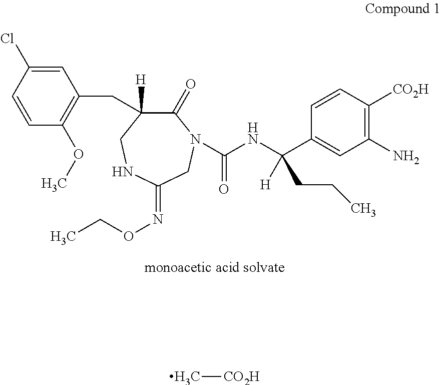

In some embodiments, the disclosure encompasses compounds of Formula I or Compound 1 or pharmaceutically acceptable salts or solvates thereof as highly selective chymase inhibitors.

In some embodiments, the disclosure encompasses a method of treating hepatobiliary diseases in a subject in need thereof, wherein the administration of a therapeutic effective amount of a compound of formula (I), including Compound 1 or pharmaceutically acceptable salts or solvates thereof, treat said disease.

In some embodiments, the disclosure encompasses a method of treating gastrointestinal diseases in a subject in need thereof, wherein the administration of a therapeutic effective amount of a compound of formula (I), including Compound 1 or pharmaceutically acceptable salts or solvates thereof, treat said disease.

In some embodiments, the disclosure encompasses a method of treating eosinophilic diseases, including Eosinophilic Esophagitis (EoE) in a subject in need thereof, wherein the administration of a therapeutic effective amount of compound of formula (I), including Compound 1 or pharmaceutically acceptable salts or solvates thereof, treat said disease.

In some embodiments, the disclosure encompasses a method of treating hepatobiliary diseases in a subject, wherein the administration of a therapeutic effective amount of a compound of formula (I), including Compound 1 or pharmaceutically acceptable salts or solvates thereof treat said disease.

In some embodiments, the disclosure encompasses a method of treating hepatobiliary diseases in a subject, wherein the administration of a therapeutic effective amount of a compound of formula (I), including Compound 1 or pharmaceutically acceptable salts or solvates thereof ameliorate pruritis.

In some embodiments, the disclosure encompasses a method of treating hepatobiliary diseases in a subject, wherein the administration of therapeutic effective amount of compound of formula (I), including Compound 1 or a pharmaceutically acceptable salt or solvate thereof ameliorates one or more of occult blood in stool, fecal incontinence, fever, fatigue, weight loss, mast cell and eosinophil counts, dysphagia, gastroesophageal reflux disease (GERD), bloating, early satiety, loss of appetite, jaundice, pruritis, hepatomegaly, cirrhosis, biliary obstruction, abdominal pain, nausea, diarrhea, spasms, and inflammation in said subject suffering from hepatobiliary disease.

In some embodiments, the disclosure encompasses a method of treating hepatobiliary diseases in a subject, wherein the administration of therapeutic effective amount of compound of formula (I) or a pharmaceutically acceptable salt or solvate thereof ameliorates inflammation.

In various embodiments, the disclosure encompasses a method of treating or preventing hepatobiliary disease in a subject in need thereof, comprising administering to the subject a therapeutic effective amount of Compound 1 or a pharmaceutically acceptable salt or solvate thereof.

In some embodiments, the present disclosure encompasses a method for reducing infiltration of mast cells in a subject suffering from hepatobiliary disease, said method comprising administering to said subject a therapeutic effective amount of compound 1 or a pharmaceutically acceptable salt or solvate thereof.

In some embodiments, the present disclosure encompasses a method for treating or preventing jaundice in a subject suffering from hepatobiliary disease, said method comprising administering to said subject a therapeutic effective amount of compound 1 or a pharmaceutically acceptable salt or solvate thereof.

In some embodiments, the present disclosure encompasses a method for treating or preventing pruritis in a subject suffering from hepatobiliary disease, said method comprising administering to said subject a therapeutic effective amount of compound 1 or a pharmaceutically acceptable salt or solvate thereof.

In some embodiments, the present disclosure encompasses a method for reducing inflammation in a subject suffering from hepatobiliary disease, said method comprising administering to said subject a therapeutic effective amount of compound 1 or a pharmaceutically acceptable salt or solvate thereof.

In some embodiments, the present disclosure encompasses a method for reducing bile acid accumulation in a subject suffering from hepatobiliary disease, said method comprising administering to said subject a therapeutic effective amount of compound 1 or a pharmaceutically acceptable salt or solvate thereof.

In some embodiments, the method treats gastrointestinal disease that may be associated with hepatobiliary disease. Such gastrointestinal disease is selected from the group consisting of acute hepatitis, alcoholic hepatitis, alcoholic liver disease, colitis, extrahepatic cholestasis, hepatic fibrosis, liver abscess, liver cirrhosis, Ulcerative colitis, Crohn's disease, Irritable bowel syndrome (IBS), Proctitis, Celiac disease, ileitis, duodenitis, diverticulitis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, non-alcoholic steatohepatitis, polycystic liver disease, veno-occlusive disease, and Wilson's disease. In embodiments, the hepatobiliary disease is PSC. In other embodiments, the hepatobiliary disease is PBC. In still other embodiments, the gastrointestinal disease is colitis.

In an embodiment, the therapeutic effective amount of compound 1 or a pharmaceutically acceptable salt or solvate thereof is from about 10 mg to about 1000 mg per unit dose (e.g., about 50 mg to about 950 mg per unit dose, about 50 mg to about 900 mg per unit dose, about 50 mg to about 850 mg per unit dose, about 50 mg to about 800 mg per unit dose, about 50 mg to about 750 mg per unit dose, about 50 mg to about 700 mg per unit dose, about 50 mg to about 650 mg per unit dose, about 50 mg to about 600 mg per unit dose, about 50 mg to about 550 mg per unit dose, about 50 mg to about 500 mg per unit dose, about 50 mg to about 450 mg per unit dose, about 50 mg to about 400 mg per unit dose, about 50 mg to about 350 mg per unit dose, about 50 mg to about 300 mg per unit dose, about 50 mg to about 250 mg per unit dose, about 50 mg to about 200 mg per unit dose, about 50 mg to about 150 mg per unit dose, about 50 mg to about 100 mg per unit dose). In embodiments, the therapeutic effective amount of compound 1 or a pharmaceutically acceptable salt or solvate thereof are administered once a day, twice a day, thrice a day, once in two days, or once in three days or once in a week.

In some embodiments, the therapeutic effective amount of compound 1 or a pharmaceutically acceptable salt thereof is administered orally to the subject in unit dosage form formulated as a pellet, multiunit particulate system (MUPS), tablets, troches, lozenges, dispersible powders or granules, or a hard or soft capsule, powders, sustained-release formulations, microcapsules.

In some embodiments, a method for treating or preventing PSC in a subject in need thereof, said method comprising administering to said subject a therapeutic effective amount of compound 1 monoacetic acid solvate.

In some embodiments, a method for treating or preventing PSC in a subject in need thereof, said method comprising administering to said subject compound 1 monoacetic acid solvate in a range of about 10 mg to 1000 mg per unit dose.

In some embodiments, the present disclosure encompasses a method for reducing angiogenesis in a subject suffering with PSC in a subject in need thereof, said method comprising administering to said subject a therapeutic effective amount of compound 1 monoacetic acid solvate.

In some embodiments, the present disclosure encompasses a method for reducing biliary senescence in a subject suffering with PSC in a subject in need thereof, said method comprising administering to said subject a therapeutic effective amount of compound 1 monoacetic acid solvate.

In some embodiments, the present disclosure encompasses a method for reducing proliferation of ductular hepatocytes in a subject suffering with PSC in a subject in need thereof, said method comprising administering to said subject a therapeutic effective amount of compound 1 monoacetic acid solvate.

In some embodiments, a method for treating or preventing PBC in a subject in need thereof, said method comprising administering to said subject a therapeutic effective amount of compound 1 monoacetic acid solvate.

In some embodiments, the present disclosure encompasses a method for treating or preventing Eosinophilic Gastro-Intestinal diseases (EGID) and/or EoE in a subject in need thereof, said method comprising administering to said subject a therapeutic effective amount of a compound of Formula (I), or a pharmaceutically acceptable salt or solvate thereof,

In some embodiments, the present disclosure encompasses a method for treating or preventing colitis in a subject in need thereof, said method comprising administering to said subject a therapeutic effective amount of compounds of the present invention, including compound 1 monoacetic acid solvate.

In some embodiments, it further encompasses a method for treating or preventing colitis in a subject in need thereof, said method comprising administering to said subject compound 1 monoacetic acid solvate in a range of about 10 mg to 1000 mg per unit dose.

In embodiments, the present disclosure encompasses a method for reducing one or more of body weight loss, diarrhea, rectal bleeding in a subject suffering with colitis in a subject in need thereof, said method comprising administering to said subject a therapeutic effective amount of compounds of the present invention, including compound 1 monoacetic acid solvate.

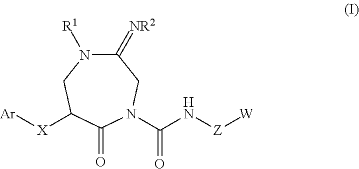

wherein,
Ar, R¹, R² Z, X, W are same as defined above.

In embodiments, the present disclosure encompasses a method for treating or preventing EGID a subject in need thereof, said method comprising administering to said subject a therapeutic effective amount of a compound 1, or a pharmaceutically acceptable salt or solvate thereof. In preferable embodiments, the compound 1 is monoacetic acid solvate.

In embodiments, the EGID is selected from the group consisting of eosinophilic gastroenteritis, eosinophilic esophagitis (EoE), eosinophilic duodenitis (EoD), eosinophilic gastritis (EoG).

In one aspect, the disclosure provides a composition comprising the compound having a structure as follows:

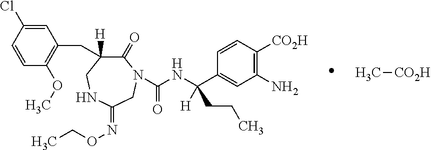

In another aspect, the disclosure provides a composition comprising 2-amino-4-[(1R-1-({[(3Z,6S)-6-(5-chloro-2-methyoxybenzyl)-3-(ethoxyimino)-7-oxo-1,4-diazepan-1-yl]carbonyl}amino)butyl]benzoic acid (compound 1) or a pharmaceutically acceptable salt or solvate thereof.

In another aspect, the disclosure provides a method for treating hepatobiliary disease in a subject in need thereof, said method comprising administering to the subject a therapeutically effective amount of 2-amino-4-[(1R-1-({[(3Z,6S)-6-(5-chloro-2-methyoxybenzyl)-3-(ethoxyimino)-7-oxo-1,4-diazepan-1-yl]carbonyl}amino)butyl]benzoic acid (compound 1) or a pharmaceutically acceptable salt or solvate thereof.

In some embodiments of any of the aforesaid methods, administration of said therapeutically effective amount of said compound 1 or a pharmaceutically acceptable salt or solvate thereof ameliorates one or more of infiltration of at least one of mast cells or macrophages, bile acid accumulation, cholangiocyte proliferation, biliary senescence, fibrosis, collagen deposition in said subject suffering from hepatobiliary disease.

In some embodiments of any of the aforesaid methods, administration of said therapeutically effective amount of said compound 1 or a pharmaceutically acceptable salt or solvate thereof ameliorates one or more of jaundice, pruritis, hepatomegaly, cirrhosis, biliary obstruction, abdominal pain, nausea, diarrhea, spasms, and inflammation in said subject suffering from hepatobiliary disease.

In some embodiments of any of the aforesaid methods, the method for treating comprises preventing hepatobiliary disease in said subject.

In another aspect, the disclosure provides a method for treating primary sclerosing cholangitis in a subject in need thereof, said method comprising administering to said subject a therapeutically effective amount of 2-amino-4-[(1R-1-({[(3Z,6S)-6(5-chloro-2-methyoxybenzyl)-3-(ethoxyimino)-7-oxo-1,4-diazepan-1-yl]carbonyl}amino)butyl]benzoic acid (compound 1) or a pharmaceutically acceptable salt or solvate thereof.

In some embodiments of any of the aforesaid methods, administration of said therapeutically effective amount of said compound 1 or a pharmaceutically acceptable salt or solvate thereof ameliorates one or more of infiltration of at least one of mast cells or macrophages, bile acid accumulation, cholangiocyte proliferation, biliary senescence, fibrosis, and collagen deposition in said subject suffering from primary sclerosing cholangitis.

In some embodiments of any of the aforesaid methods, administration of said therapeutically effective amount of said compound 1 or a pharmaceutically acceptable salt or solvate thereof ameliorates one or more of occult blood in stool, fecal incontinence, fever, fatigue, weight loss, mast cell and eosinophil counts, dysphagia, gastroesophageal reflux disease (GERD), bloating, early satiety, loss of appetite, jaundice, pruritis, hepatomegaly, cirrhosis, biliary obstruction, abdominal pain, nausea, diarrhea, spasms, and inflammation in said subject suffering from PSC.

In some embodiments of any of the aforesaid methods, the method for treating comprises preventing PSC in said subject.

In some embodiments of any of the aforesaid methods, said administering of said therapeutically effective amount of said compound 1 or a pharmaceutically acceptable salt or solvate thereof treats at least one of inflammatory bowel disease and colitis in said subject.

In another aspect, the disclosure provides a method for treating PBC in a subject in need thereof, said method comprising administering to the subject a therapeutically effective amount of 2-amino-4-[(1R-1-({[(3Z,6S)-6(5-chloro-2-methyoxybenzyl)-3-(ethoxyimino)-7-oxo-1,4-diazepan-1-yl]carbonyl}amino)butyl]benzoic acid (compound 1) or a pharmaceutically acceptable salt or solvate thereof.

In some embodiments of any of the aforesaid methods, administration of said therapeutically effective amount of said compound 1 or a pharmaceutically acceptable salt or solvate thereof ameliorates one or more of infiltration of at least one of mast cells or macrophages, bile acid accumulation, cholangiocyte proliferation, biliary senescence, fibrosis, and collagen deposition in said subject suffering from primary biliary cholangitis.

In some embodiments of any of the aforesaid methods, administration of said therapeutically effective amount of said compound 1 or a pharmaceutically acceptable salt or solvate thereof ameliorates one or more of occult blood in stool, fecal incontinence, fever, fatigue, weight loss, mast cell and eosinophil counts, dysphagia, gastroesophageal reflux disease (GERD), bloating, early satiety, loss of appetite, jaundice, pruritis, hepatomegaly, cirrhosis, biliary obstruction, abdominal pain, nausea, diarrhea, Ulcerative colitis, Crohn's disease, Irritable bowel syndrome (IBS), Proctitis, Celiac disease, ileitis, duodenitis, diverticulitis, spasms, and inflammation in said subject suffering from primary biliary cholangitis.

In some embodiments of any of the aforesaid methods, the method for treating comprises preventing PBC in said subject.

In some embodiments of any of the aforesaid methods, said administering of said therapeutically effective amount of said compound 1 or a pharmaceutically acceptable salt or solvate thereof treats at least one of inflammatory bowel disease and colitis in said subject.

In some embodiments of any of the aforesaid methods, the compound of formula (I)1 or a pharmaceutically acceptable salt or solvate thereof is a selective for chymase over cathepsin G. The compound of Formula I has an IC50 value of 0.029 μM with respect to human chymase whereas the IC50 value is 32 μM for Cathepsin. Hence the compound of Formula I is extremely selective and specific for inhibition of chymase.

In some embodiments of any of the aforesaid methods, the hepatobiliary disease is selected from the group consisting of acute hepatitis, alcoholic hepatitis, alcoholic liver disease, extrahepatic cholestasis, hepatic fibrosis, liver abscess, liver cirrhosis, colitis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, non-alcoholic steatohepatitis, polycystic liver disease, veno-occlusive disease, and Wilson's disease.

In other embodiments, a gastrointestinal disease is selected from the group consisting of Ulcerative colitis, Crohn's disease, Irritable bowel syndrome (IBS), Proctitis, Celiac disease, ileitis, duodenitis, and diverticulitis.

In some embodiments of any of the aforesaid methods, therapeutically effective amount of said compound 1 or a pharmaceutically acceptable salt or solvate thereof is from about 20 mg to about 400 mg per unit dose. In some embodiments, therapeutically effective amount of said compound 1 or a pharmaceutically acceptable salt or solvate thereof is from about 20 mg, 25 mg, 30 mg, 40 mg, 50 mg, 75 mg, 100 mg, 125 mg, 150 mg, 175 mg, 200 mg, 225 mg, 250 mg, 275 mg, 300 mg, 325 mg, 350 mg, 375 mg and 400 mg.

In some embodiments of any of the aforesaid methods, the therapeutically effective amount of said compound 1 or a pharmaceutically acceptable salt or solvate thereof is administered orally to the subject once a day, twice a day, thrice a day, once in two days, once in three days, or once a week.

In some embodiments of any of the aforesaid methods, said compound 1 is:

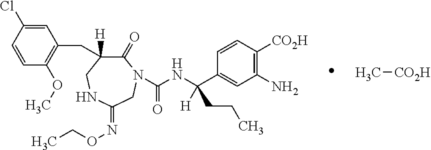

In one aspect, the disclosure provides a method for treating at least one of primary sclerosing cholangitis and primary biliary cholangitis in a subject in need thereof, said method comprising administering to the subject a therapeutically effective amount of a compound of Formula (I), or a pharmaceutically acceptable salt or solvate thereof:

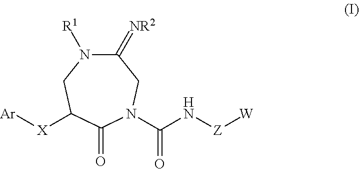

wherein,
Ar is (1) a C6 to C14 aromatic hydrocarbon group, (2) a 5 to 8-membered aromatic heterocyclic group having 1 to 4 hetero atoms selected from the group consisting of a nitrogen atom, sulfur atom, and oxygen atom, in place of a carbon atom, or (3) a bicyclic or tricyclic aromatic group formed by condensation of the above aromatic heterocyclic group and a C6 to C14 aromatic hydrocarbon ring,
where the above groups (1) to (3) of Ar are optionally substituted with 1 to 5 groups selected from the group consisting of:
(viii) a halogen atom,
(ix) nitro,
(x) cyano,
(xi) C1 to C6 alkyl optionally substituted with 1 to 3 halogen atoms,
(xii) C2 to C6 alkenyl optionally substituted with 1 to 3 halogen atoms,
(xiii) C2 to C6 alkynyl optionally substituted with 1 to 3 halogen atoms,
(xiv) C3 to C6 cycloalkyl,
(ix) hydroxyl,
(xiii) C1 to C6 alkoxy optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(xiv) C1 to C5 alkylenedioxy,
(xv) C1 to C6 alkylthio optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(xvi) amino,
(xix) mono-C1 to C6 alkylamino,
(xx) di-C1 to C6 alkylamino,
(xxi) 5- to 6-membered cyclic amino,
(xxii) C1 to C6 alkylcarbonyl,
(xxiii) carboxyl,
(xxiv) C1 to C6 alkoxycarbonyl,
(xxviii)carbamoyl,
(xxix) thiocarbamoyl,
(xxx) mono-C1 to C6 alkylcarbamoyl,
(xxxi) di-C1 to C6 alkylcarbamoyl,
(xxxii) 5- to 6-membered cyclic aminocarbonyl,
(xxxiii) sulfo,
(xxxiv) C1 to C6 alkylsulfonyl,
(xxxv) C1 to C6 alkoxycarbonylamino,
(xxxvi) C1 to C6 alkylcarbonylamino,
(xxxi) mono- or di-C1 to C6 alkylaminocarbonylamino,
(xxxii) aminosulfonyl, and
(xxxiii) mono- or di-C1 to C6 alkylaminosulfonyl, or substituted with 1 to 9 deuterium atoms,
X is (1) a connecting bond, (2) linear or branched C1 to C6 alkylene optionally substituted with
1 to 12 deuterium atoms, (3) an oxygen atom, (4) NR3, where R3 is a hydrogen atom or a C1 to
C6 alkyl group, or (5) —S(O)m-, where m is an integer of 0 to 2,
Z is (1) a connecting bond or (2) CR4R5 where R4 and R5 are, independently,
(G) a hydrogen atom,
(H) a deuterium atom,
(I) C1 to C6 alkyl optionally substituted with 1 to 5 groups selected from the group consisting of (i) carboxyl, (ii) C1 to C6 alkoxycarbonyl, (iii) phenyl, (iv) hydroxyl, (v) C1 to C6 alkoxy, (vi) halogen atom, and (vii) C3 to C6 cycloalkyl, or substituted with 1 to 13 deuterium atoms,
(J) C3 to C6 cycloalkyl optionally substituted with 1 to 5 groups selected from the group consisting of (i) a halogen atom and (ii) an alkyl group optionally substituted with 1 to 3 halogen atoms or optionally substituted, with 1 to 11 deuterium atoms,
(K) COOR6, where R6 is a hydrogen atom or C1 to C6 alkyl, or
(L) CONR7R8, where R7 and R8 are, independently,
(d) hydrogen atom,
(e) C1 to C6 alkyl optionally substituted with 1 to 3 groups selected from the group consisting of (i) a halogen atom, (ii) C3 to C6 cycloalkyl, (iii) carboxyl, (iv) C1 to C6 alkoxycarbonyl, (v) C1 to C6 alkylcarbonyl, (vi) carbamoyl, (vii) mono-C1 to C6 alkylcarbamoyl, (viii) di-C1 to C6 alkylcarbamoyl, (ix) C6 to C12 aryl, and (x) C1 to C10 heteroaryl,
(f) C6 to C14 aromatic hydrocarbon group,
(f) a 5- to 8-membered aromatic heterocyclic group having 1 to 4 hetero atoms selected from the group consisting of a nitrogen atom, sulfur atom, and oxygen atom, in place of a carbon atom, or
(g) a bicyclic or tricyclic aromatic group formed by condensation of the above aromatic heterocyclic, group and a C6 to C14 aromatic hydrocarbon ring,
where each of the groups (c) to (e) is optionally substituted with 1 to 5 groups selected from the group consisting of (i) a halogen atom, (ii) nitro, (iii) cyano, (iv) C1 to C6 alkyl optionally substituted with 1 to 3 halogen atoms, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 halogen atoms, (x) C1 to C5 alkylenedioxy, (xi) C1 to C6 alkylthio optionally substituted with 1 to 3 halogen atoms, (xii) amino, (xiii) mono-C1 to C6 alkylamino, (xiv) di-C1 to C6 alkylamino, (xv) 5- to 6-membered cyclic amino, (xvi) C1 to C6 alkylcarbonyl, (xvii) carboxyl, (xviii) C1 to C6 alkoxycarbonyl, (xix) carbamoyl, (xx) thiocarbamoyl, (xxi) mono-C1 to C6 alkylcarbamoyl, (xxii) di-C1 to C6 alkylcarbamoyl, (xxiii) C6 to C10 arylcarbamoyl, (xxiv) C1 to C10 heteroarylcarbamoyl, (xxv) sulfo, (xxvi) C1 to C6 alkylsulfonyl, (xxvii) aminosulfonyl, and (xxviii) mono- or di-C1 to C6 alkylaminosulfonylamino, or substituted with 1 to 9 deuterium atoms, W is (1) a hydrogen atom, (2) a C6 to C14 aromatic hydrocarbon group, (3) a 5- to 8-membered aromatic heterocyclic group having 1 to 4 hetero atoms selected from a nitrogen atom, a sulfur atom, and an oxygen atom, in place of a carbon atom, (4) a bicyclic, or tricyclic aromatic group formed by condensation of the above aromatic heterocyclic group and a C6 to C14 aromatic hydrocarbon ring, or a deuterium atom,
where, each of the groups (2) to (4) of the above W is optionally substituted with 1 to 5 groups selected from the group consisting of (i) a halogen atom, (ii) nitro, (iii) cyano, (iv) C1 to C6 alkyl optionally substituted with 1 to 3 groups selected from the group consisting of a halogen atom, amino, C1 to C6 alkoxycarbonyl, C1 to C6 alkoxycarbonylamino, and carboxyl, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 groups selected from the group consisting of a halogen atom, hydroxyl, C1 to C6 alkoxy, amino and mono- or di-C1 to C6 alkylamino, (x) C1 to C5 alkylenedioxy, (xi) C1 to C6 alkylthio optionally substituted with 1 to 3 groups selected from the group consisting of a halogen atom, hydroxyl, C1 to C6 alkoxy, amino, and mono- or di-C1 to C6 alkylamino, (xii) amino, (xiii) mono-C1 to C6 alkylamino, (xiv) di-C1 to C6 alkylamino,
(xv) 5- to 6-membered cyclic amino, (xvi) C1 to C6 alkyl-carbonyl, (xvii) carboxyl, (xviii) C1 to C6 alkoxycarbonyl optionally substituted with a halogen atom, (xix) C7 to C16 aralkyloxycarbonyl optionally substituted with a halogen atom, (xx) carbamoyl, (xxi) mono-C1 to C6 alkylcarbamoyl optionally substituted with 1 to 3 groups which are selected from the group consisting of a halogen atom, hydroxyl, carboxyl, C1 to C6 alkoxy, amino, and mono- or di-C1 to C6 alkylamino, (xxii) di-C1 to C6 alkylcarbamoyl optionally substituted with hydroxyl, (xxiii) 5- to 6-membered cyclic aminocarbonyl optionally substituted with C1 to C6 alkoxycarbonyl, (xxiv) C6 to C10 arylcarbamoyl, (xxv) C1 to C10 heteroarylcarbamoyl, (xxvi) C7 to C16 aralkylcarbamoyl, (xxvii) C1 to C10 heteroaryl-C1 to C6 alkylcarbamoyl, (xxviii) N C1 to C6 alkyl-N—C6 to C12 arylcarbamoyl, (xxix) C3 to C6 cycloalkylcarbamoyl, (xxx) sulfo, (xxxi) C1 to C6 alkylsulfonyl, (xxxii) C1 to C6 alkylsulfonylamino, (xxxiii) C6 to C12 arylsulfonylamino optionally substituted with C1 to C6 alkyl, (xxxiv) C1 to C10 heteroarylsulfonylamino, (xxxv) C1 to C6 alkoxycarbonylamino, (xxxvi) C1 to C6 alkylcarbonylamino, (xxxvii) mono- or di-C1 to C6 alkylaminocarbonylamino, (xxxviii) C6 to C12 aryl, (xxxix) C1 to C10 heteroaryl, (xl) C6 to C10 aryloxy, (xli) C1 to C10 heteroaryloxy, (xlii) C7 to C16 aralkyloxy, (xliii) C1 to C10 heteroaryl-C1 to C6 alkyloxy, (xliv) aminosulfonyl, (xlv) mono- or di-C1 to C6 alkylaminosulfonyl, (xlvi) C7 to C16 aralkyloxycarbamoyl, and (xlvii) C1 to C10 heteroaryl-C1 to C6 alkyloxycarbamoyl, or substituted with 1 to 9 deuterium atoms,
R1 is
(6) a hydrogen atom,
(7) C1 to C6 alkyl optionally substituted with 1 to 3 of (i) a halogen atom or (ii) C3 to C6 cycloalkyl or substituted with (iii) 1 to 13 deuterium atoms,
(8) C2 to C6 alkenyl optionally substituted with 1 to 3 of (i) a halogen atom or (ii) C3 to C6 cycloalkyl or substituted with (iii) 1 to 11 deuterium atoms,
(9) C2 to C6 alkynyl optionally substituted with 1 to 3 of (i) a halogen atom or (ii) C3 to C6 cycloalkyl, or substituted with (iii) 1 to 9 deuterium atoms, or
(10) C3 to C6 cycloalkyl optionally substituted with 1 to 3 of (i) a halogen atom or (ii) C3 to C6 cycloalkyl, or substituted with (iii) 1 to 11 deuterium atoms, R² is (1) OR9 or (2) NR10R11
where, R9, R10, and R11 independently indicate
(E) a hydrogen atom,
(F) C1 to C6 alkyl,
(G) C2 to C6 alkenyl,
(H) C3 to C6 alkynyl,
(I) C3 to C6 cycloalkyl
(J) C6 to C14 aromatic hydrocarbon group,
(K) a 5- to 8-membered aromatic heterocyclic group having 1 to 4 hetero atoms selected from the group consisting of a nitrogen atom, sulfur atom, and oxygen atom, in place of a carbon atom, or
(L) a bicyclic or tricyclic aromatic group formed by condensation of the above aromatic heterocyclic group and a C6 to C14 aromatic hydrocarbon ring,
where each of the groups of the above (B) to (E) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5 to 6-membered cyclic amino, (ix) carboxyl, (x) C1 to C6 alkoxycarbonyl, (xi) C1 to C6 alkylcarbonyl, (xii) carbamoyl, (xiii) mono-C1 to C6 alkylcarbamoyl, (xiv) di-C1 to C6 alkylcarbamoyl, (xv) C6 to C12 aryl, and (xvi) C1 to C10 heteroaryl, or substituted with 1 to 13 deuterium atoms, further, each of the groups of the above (F) to (H) are optionally substituted with 1 to 5 groups which are selected from the group consisting of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms, or R1 and R2 may form 5- or 6-member heterocyclic ring together with the atoms they are bonded with, where the above 5- or 6-member heterocyclic ring is optionally substituted with 1 to 3 groups selected from the group consisting of
(I) a halogen atom,
(J) oxo,
(K) hydroxyl
(L) C1 to C6 alkyl,
(M) C2 to C6 alkenyl,
(N) C2 to C6 alkynyl,
(O) C3 to C6 cycloalkyl
(P) C6 to C14 aromatic hydrocarbon group,
(L) 5- to 8-membered aromatic heterocyclic group having 1 to 4 hetero atoms selected from the group consisting of a nitrogen atom, sulfur atom, and oxygen atom, in place of a carbon atom, and
(M) a bicyclic or tricyclic aromatic group formed by condensation of the above aromatic heterocyclic group and a C6 to C14 aromatic hydrocarbon ring, or substituted with
(N) 1 to 6 deuterium atoms,
where each of the groups of the above (D) to (G) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5- to 6-membered cyclic amino, (ix) carboxyl, (x) C1 to C6 alkoxycarbonyl, (xi) C1 to C6 alkyl-carbonyl, (xii) carbamoyl, (xiii) mono-C1 to C6 alkylcarbamoyl, (xiv) di-C1 to C6 alkylcarbamoyl, (xv) C6 to C12 aryl and (xvi) C1 to C10 heteroaryl, or substituted with 1 to 13 deuterium atoms, each of the groups of the above (H) to (J) is optionally substituted with 1 to 5 groups which are selected from the group consisting, of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms.

In some embodiments of any of aforesaid methods, administration of said therapeutically effective amount of compound of Formula I or a pharmaceutically acceptable salt or solvate thereof ameliorates one or more of infiltration of at least one of mast cells or macrophages, bile acid accumulation, cholangiocyte proliferation, biliary senescence, fibrosis, collagen deposition in said subject suffering from hepatobiliary disease.

In some embodiments of any of aforesaid methods, administration of said therapeutically effective amount of compound of Formula I or a pharmaceutically acceptable salt or solvate thereof ameliorates one or more of occult blood in stool, fecal incontinence, fever, fatigue, weight loss, mast cell and eosinophil counts, dysphagia, gastroesophageal reflux disease (GERD), bloating, early satiety, loss of appetite, jaundice, pruritis, hepatomegaly, cirrhosis, biliary obstruction, abdominal pain, nausea, diarrhea, spasms, Ulcerative colitis, Crohn's disease, Irritable bowel syndrome (IBS), Proctitis, Celiac disease, ileitis, duodenitis, diverticulitis, and inflammation in said subject suffering from hepatobiliary disease.

In some embodiments of any of aforesaid methods, the compound of formula (I) or a pharmaceutically acceptable salt or solvate thereof is a selective for chymase over cathepsin G.

In some embodiments of any of aforesaid methods, the hepatobiliary disease is selected from the group consisting of acute hepatitis, alcoholic hepatitis, alcoholic liver disease, extrahepatic cholestasis, hepatic fibrosis, liver abscess, liver cirrhosis, colitis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, non-alcoholic steatohepatitis, polycystic liver disease, veno-occlusive disease, and Wilson's disease.

In some embodiments of any of aforesaid methods, said therapeutically effective amount of compound of Formula I or a pharmaceutically acceptable salt or solvate thereof is from about 20 mg to about 400 mg per unit dose.

### BRIEF DESCRIPTION OF THE DRAWINGS

FIG. 1: illustrates the decrease Chymase and Tryptase positive mast cells with 20 mg/kg compound 1 monoacetic acid solvate in Mdr2 Knock Out (Mdr2 KO) mice.

FIG. 2: illustrates the decrease in Liver and Serum TBA with 20 mg/kg compound 1 monoacetic acid solvate in Mdr2 Knock Out (Mdr2 KO) mice.

FIG. 3: illustrates the decrease in the levels of mast cell markers (Mcpt1, Fcer1a, Tpsb2) with compound 1 monoacetic acid solvate in Mdr2 Knock Out (Mdr2 KO) mice.

FIG. 4: illustrates the decrease in Macrophage marker (F4/80) with compound 1 monoacetic acid solvate in Mdr2 Knock Out (Mdr2 KO) mice.

FIG. 5: illustrates the decrease in Cholangiocyte activation marker (Desmin) with compound 1 monoacetic acid solvate in Mdr2 Knock Out (Mdr2 KO) mice.

FIG. 6: illustrates the decrease in collagen deposition with compound 1 monoacetic acid solvate in Mdr2 Knock Out (Mdr2 KO) mice.

FIG. 7: illustrates the decrease in Hydroxyproline levels with compound 1 monoacetic acid solvate in Mdr2 Knock Out (Mdr2 KO) mice.

FIG. 8: illustrates the decrease in the levels of Fibrosis markers (Cola1, Timp2, Tgfb1) with compound 1 monoacetic acid solvate in Mdr2 Knock Out (Mdr2 KO) mice

FIG. 9: illustrates the decrease in CK-19 levels with compound 1 monoacetic acid solvate in Mdr2 Knock Out (Mdr2 KO) mice

FIG. 10: illustrates the decrease in levels of Biliary senescence markers (Cdkn2a, Cdkn2c, Tgfb1, Glb1l) with compound 1 monoacetic acid solvate in Mdr2 Knock Out (Mdr2 KO) mice

FIG. 11: illustrates the decrease in Body weight loss score with 20 mg/kg compound 1 monoacetic acid solvate in a DSS-induced colitis mouse model.

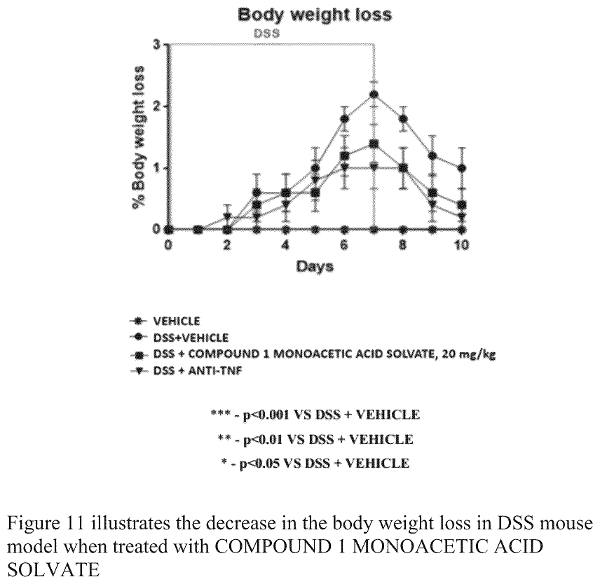

FIG. 12: illustrates the decrease in Occult blood score with 20 mg/kg compound 1 monoacetic acid solvate in a DSS-induced colitis mouse model.

FIG. 13: illustrates the decrease in Stool consistency score with 20 mg/kg compound 1 monoacetic acid solvate in a DSS-induced colitis mouse model.

FIG. 14: illustrates the decrease in DAI score with 20 mg/kg compound 1 monoacetic acid solvate in a DSS-induced colitis mouse model.

FIG. 15: illustrates the improvement in stool consistency score, DAI, Occult blood score percentage body weight loss in the DSS-induced colitis mouse model. Administration of compound 1 has attenuated the symptoms of DSS-induced colitis in mice.

### DETAILED DESCRIPTION OF THE INVENTION

In the following passages, different embodiments of the disclosure are defined in more detail. Each embodiment so defined may be combined with any other aspect or aspects unless clearly indicated to the contrary. In particular, any feature indicated as being preferred or advantageous may be combined with any other feature or features indicated as being preferred or advantageous.

### I. Abbreviations

As used herein, the following abbreviations have the following meanings:
mg: milligram
i.p.: intraperitoneal
OD: once daily
PBC: Primary Biliary cholangitis/primary biliary cirrhosis
PSC: Primary sclerosing cholangitis
MC: Mast cells
Mdr2-Multidrug resistance 2 gene, also known as adenosine triphosphate-binding cassette B4 (ABCB4)
MUPS: multiunit particulate system
IBD: Inflammatory bowel disease
UC: Ulcerative Colitis
CD: Crohn's disease
GI: Gastro-intestinal
GBA: Gut Brain Axis
MMP-9: matrix metalloproteinase 9
TGF: transforming growth factor
SCF: Stem Cell Factor
EGID: Eosinophilic Gastrointestinal disease
BDL: Bile duct ligated
TBA: total bile acid
NAD: Nicotinamide adenine dinucleotide
EIA: enzyme immunoassay
KO: knock out
WT: Without treatment
TNF: tumor necrosis factor
IL-1b: Interleukin 1b
IL-18: Interleukin 18
IL-6: Interleukin 6
MCP-1: Mast cell protease-
IL-33: Interleukin 33

### II. Definitions

It will be understood that the terminology used herein is for the purpose of describing embodiments only and is not intended to be limiting. As used in this specification, the singular forms “a”, “an” and “the” include plural referents unless the context clearly dictates otherwise.

As used herein, the term “about” refers to ±10% of the indicated numerical value unless otherwise stated or otherwise evident by the context, and except where such a range would exceed 100% of a possible value, or fall below 0% of a possible value, such as less than 0% content of an ingredient, or more than 100% of the total contents of a composition.

As used herein, the term “solvate” refers to a crystal form containing the compound of formula (I) or a pharmaceutically acceptable salt thereof and either a stoichiometric or a non-stoichiometric amount of a solvent. Solvents, by way of example, include water, methanol, ethanol, 1-propanol, 2-propanol, formic acid, ethyl formate, acetic acid, methyl acetate, ethyl acetate, propyl acetate and isobutyl acetate, etc. Hereinafter, reference to a compound of formula (I) is to any physical form of that compound, unless a particular form, salt or solvate thereof is specified.

As used herein, the term “active agent” or “therapeutic agent” refers to a chemical agent capable of activity.

As used herein, the terms “comprises”, “comprising”, “includes”, “including”, “having” means “including but not limited to”.

As used herein, the terms “formulation” and “composition” are used interchangeably, except where otherwise clearly intended to have different meanings.

As used herein, the term “Hepatobiliary disease” refers to a medical condition in which bile excretion from the liver is impaired or blocked. Clinical symptoms and signs include fatigue, jaundice, itching (pruritis) fibrosis and inability to digest certain foods, nausea, vomiting, dark and pale urine. Hepatobiliary disease according to the present teachings refers to any stage or severity of Hepatobiliary disease (e.g., disease remission or acute disease).

As used herein, the term “Eosinophilic Gastrointestinal disease” refers to a medical condition in which eosinophilic count increases in any part of the gastro-intestinal tract from esophagus to colon. EGID according to the present teachings refers to any stage or severity of Hepatobiliary disease (e.g., disease remission or acute disease).

The disclosure also includes all stereoisomers of the compounds, including diastereomers and enantiomers. The disclosure also includes mixtures of stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

As used herein, the term “inhibit,” refers to the ability of a compound or any agent to reduce or impede a described function, level, activity, synthesis, release, binding, etc., based on the context in which the term “inhibit” is used. The term “inhibit” is used interchangeably with “reduce,” “block,” and “decrease.”

As used herein, the terms “patient” or “subject” refer to a living organism suffering from or prone to a condition that can be treated by administration of a pharmaceutical composition as provided herein. Non-limiting examples include humans, other mammals and other non-mammalian animals.

As used herein, the term “pharmaceutically acceptable salt” refers to a salt known to be non-toxic and commonly used in the pharmaceutical literature. When the compound having the formula (I) has an amine or other basic group as a substituent group, for example, salts with hydrochloric acid, hydrobromic acid, sulfuric acid, phosphoric acid, and other mineral acids, salts with formic acid, acetic acid, fumaric acid, maleic acid, oxalic acid, citric acid, malic acid, tartaric acid, aspartic acid, glutamic acid, and other organic carboxylic acids or salts with methanesulfonic acid, benzenesulfonic acid, p-toluenesulfonic acid, hydroxybenzenesulfonic acid, and other sulfonic acids, etc. may be mentioned. When the compound having the formula (I) has a carboxylic acid or other acid group as a substituent group, for example, sodium, potassium, or other alkali metal salt, calcium, magnesium or other alkali earth metal salt, a salt with or ammonia, triethylamine, or ethylenediamine, propanediamine, pyrrolidine, piperidine, piperadine, pyridine, lysine, choline, ethanolamine, N,N-dimethylethanolamine, 4-hydroxypiperidine, glucosamine, N-methylglucamine, or other organic bases may be mentioned and droxyl alkandioic acids, aromatic acids, aliphatic and aromatic sulfonic acids may also be used.

As used herein, the terms “pharmaceutically acceptable excipient” and “pharmaceutically acceptable carrier” refer to a substance that aids the administration of an active agent to and absorption by a subject. It also refers to an excipient that can be included in the compositions of the disclosure and that causes no significant adverse toxicological effect on the patient.

As used herein the term “infiltration of mast cells” refers to the increase in the mast cells into the liver resulting in hepatobiliary diseases.

As used herein the term “inflammation” refers to the sign such as redness, swelling, when a body responds to an irritant.

As used herein the term “jaundice” refers to the medical condition in which skin, mucous membrane, white of eyes turns yellow due to increase in the level of bilirubin.

As used herein the term “pruritis” refers to the itchy medical condition

As used herein the term “angiogenesis” refers to the formation of blood vessels. as a vital process for healing wounds.

As used herein the term “biliary senescence” refers to the pathophysiological phenomenon in which proliferative cells enter cell cycle arrest following DNA damage and other stress signals. Natural, permanent DNA damage can occur after repetitive cell division; however, acute stress or other injuries can push cells into premature senescence and eventually a senescence-associated secretory phenotype (SASP).

As used herein the term “bile acid accumulation” refers to the accumulation of bile acid in liver due to impaired blood flow. It refers to the accumulation of toxic bile acids in the hepatobiliary system which damages bile duct epithelial cells and hepatocytes, causing liver injury and inflammation. Excess accumulation of bile acids leads to Chronic cholestasis which then progresses to fibrosis, cirrhosis and eventually liver failure or hepatocellular or cholangiocellular carcinomas.

As used herein the term “proliferation of ductular hepatocytes” refers to the cellular hepatic reactions that occurs in chronic liver disease. resulting in increase in bile duct mass.

As used herein, the term “preventing” or “prevention” means preventing the occurrence of a disease, condition, or associated symptoms or preventing the recurrence of the same, for example after a period of improvement.

As used herein, the term “Fibrosis” refers to the development of fibrous connective tissue as a reparative response to injury or damage. Fibrosis may refer to the connective tissue deposition that occurs as part of normal healing or to the excess tissue deposition that occurs as a pathological process.

As used herein, the term “Cholangiocyte activation” refers to the process by Cholangiocytes are activated which could occur through one or more of infections, cholestasis, ischaemia and xenobiotics. In many cholangiopathies, PSC and PBC included, the activating insult is unknown. Features that characterize the activated cholangiocyte include increased proliferation and pro-fibrotic and pro-inflammatory secretions. Activated cholangiocytes are also involved in recruitment and crosstalk with immune, vascular and mesenchymal cells, and upon chronic activation the development of biliary fibrosis and cholangiocarcinoma. The broad changes in protein expression and the activated cholangiocyte secretome make the cholangiocyte an active participant in ongoing immunological reactions to biliary injury through pleiotropic autocrine and paracrine mechanisms.

As used herein, the term “therapeutic effective amount” refers to the amount of active agent or pharmaceutical agent that elicits the biological or medicinal response in a tissue, system, animal, individual or human that is being sought by a researcher, veterinarian, medical doctor or other clinician or caregiver or by an individual, which includes one or more of the following:
(1) Preventing the disease, for example, preventing a disease, condition or disorder in an individual that may be predisposed to the disease, condition or disorder but does not yet experience or display the pathology or symptomatology of the disease;
(2) Inhibiting the disease, for example, inhibiting a disease, condition or disorder in an individual that is experiencing or displaying the pathology or symptomatology of the disease, condition or disorder (i.e., arresting further development of the pathology and/or symptomatology); and
(3) Ameliorating the disease, for example, ameliorating a disease, condition or disorder in an individual that is experiencing or displaying the pathology or symptomatology of the disease, condition or disorder (i.e., reversing the pathology and/or symptomatology).

As used herein the term “treatment” refers to one or more of the following:
(1) prevention of a disease, for example, prevention of a disease, condition or disorder in an individual that may be predisposed to the disease, condition or disorder but does not yet experience or display the pathology or symptomatology of the disease;
(2) inhibition of a disease, for example, inhibition of a disease, condition or disorder in an individual that is experiencing or displaying the pathology or symptomatology of the disease, condition or disorder (i.e., arresting further development of the pathology and/or symptomatology); and
(3) amelioration of a disease, for example, amelioration of a disease, condition or disorder in an individual that is experiencing or displaying the pathology or symptomatology of the disease, condition or disorder (i.e., reversing the pathology and/or symptomatology).

As used herein, the term “unit dosage form” refers to physically discrete units suitable as unitary dosages for human subjects and other mammals (e.g., dogs), each unit containing a predetermined quantity of active material calculated to produce the desired onset, tolerability, and/or therapeutic effects, in association with a suitable pharmaceutical excipient (e.g., a tablet or capsule).

“C6 to C14 aromatic hydrocarbon group” expressed by Ar, a monocyclic or polycyclic aromatic hydrocarbon group, more specifically, phenyl, biphenyl, naphthyl, indenyl, anthryl, phenanthryl (preferably, phenyl, biphenyl, naphthyl, etc., particularly preferably phenyl etc.), or other 6- to 14-membered monocyclic or polycyclic aromatic hydrocarbon group etc. may be mentioned.

Further, as examples of the “5- to 8-membered aromatic heterocyclic group including 1 to 4 hetero atoms selected from a nitrogen atom, sulfur atom, and oxygen atom, other than a carbon atom” expressed by Ar, for example, a monocyclic group including 1 or more (e.g., 1 to 4, preferably 1 to 3) hetero atoms which preferably consist of 1 or 2 species of hetero atoms selected from, other than a carbon atom, a nitrogen atom, oxygen atom, and sulfur atom, or its condensed aromatic heterocyclic group, more specifically, thienyl, furyl, pyrrolyl, imidazolyl, pyrazolyl, triazolyl, tetrazolyl, thiazolyl, isothiazolyl, oxazolyl, isoxazolyl, pyridyl, pyradinyl, pyrimidinyl, pyridazinyl, naphthylidinyl, purinyl, and other aromatic heterocyclic groups (preferably pyridyl, thienyl, and furyl) etc. may be mentioned.

Further, as the “bicyclic or tricyclic aromatic group formed by condensation of the above aromatic heterocyclic group and a C6 to C14 aromatic hydrocarbon group” represented by Ar, benzothienyl, benzofuryl, indolyl, isoindolyl, benzimidazolyl, benzopyrazolyl, benzotriazolyl, benzothiazolyl, benzisothiazolyl, benzoxazolyl, benzisoxazolyl, benzodioxolyl, quinolyl, isoquinolyl, quinoxalinyl, phthalazinyl (preferably, benzothienyl, benzofuryl, benzodioxolyl, and quinolyl), etc. may be mentioned.

As used herein “compound” meant to include all stereoisomers, geometric isomers, tautomers, and isotopes of the structures depicted. Compounds herein identified by name or structure as one particular tautomeric form are intended to include other tautomeric forms unless otherwise specified.

As used herein “SASP” refers to Senescence-associated secretory phenotype (SASP), which is a phenotype associated with senescent cells wherein those cells secrete high levels of inflammatory cytokines, immune modulators, growth factors, and proteases. SASP factors can be globally divided into the following major categories: soluble signaling factors (interleukins, chemokines, and growth factors), secreted proteases, and secreted insoluble proteins/extracellular matrix (ECM) components.

As used herein, the term “Primary biliary cholangitis” also known as “Primary biliary cirrhosis” (PBC) refers to chronic liver disease resulting from progressive destruction of the bile ducts in the liver. The disease is often discovered incidentally due to abnormal results on routine liver blood tests. Once Primary Biliary Cholangitis (PBC) is suspected, a blood test to check for antimitochondrial antibody (AMA) may be done. This test is positive in nearly all people with Primary Biliary Cholangitis (PBC). A liver biopsy, where a small sample of liver tissue is removed with a small needle can help confirm the diagnosis. Various co-morbidities may be incident to PBC and PSC, including for example, inflammatory bowel disease and colitis.

As used herein, the term “Primary sclerosing cholangitis” (PSC) is a chronic liver disease in which the bile ducts inside and outside the liver become inflamed and scarred, and eventually narrowed or blocked. This results in the buildup of bile in the liver and causing further liver damage. PSC causes progressive fibrosing, inflammatory destruction of intrahepatic and extrahepatic bile ducts and subsequent cholestasis. Patients often progress to cirrhosis needing a liver transplant. Genetic susceptibility, environmental triggers, compromised gut/liver axis, abnormal composition of the gut flora and over-production of bile/bile acids are believed to be some of the causes for PSC. It is often characterized by the presence of overactivated interactions between cholangiocytes and immune cells such as mast cells. (Vesterhus. J Hep. 2017; 67:6, 1298-1323).

As used herein, the term “Ductular reaction” (DR) refers to an array of protein changes in liver tissue in response to either acute or chronic injury. It is characterized by the proliferation of reactive bile ducts induced by liver injuries. DR is pathologically recognized as bile duct hyperplasia and is commonly observed in biliary disorders.

As used herein, the term “Mast cells” (MC) refers to that is found in connective tissues all through the body, especially under the skin, near blood vessels and lymph vessels, in nerves, and in the lungs and intestines. Mast cells play an important role in how the immune system responds to certain bacteria and parasites and they help control other types of immune responses. Mast cells secrete chemokines and other factors (e.g., histamine) promoting an inflammatory response. In human PSC, MCs infiltrate the liver and contribute to liver damage. MCs interact and reside near cholangiocytes. Evidence from the scientific literature suggests an important role of mast cells and chymase in eosinophilia. For example, gastric and duodenal biopsy specimens from patients with EoE and/or EG/EoD exhibited a significant increase in mean mast cell counts compared with biopsy specimens from patients without the disease. The mean mast cell count also correlates very well with the eosinophil counts observed in the esophageal and duodenal biopsy specimens of EoE patients. In addition, the higher mast cell counts has been associated with the presence of several EG/EoD symptoms (e.g., dysphagia, abdominal pain, diarrhea) and endoscopic features (i.e., duodenal erosions and granularity).

Chymase as an important mediator released after mast cell degradation has been shown to promote viability, infiltration and activation of eosinophils in these conditions. Chymase inhibition is thus an attractive target for EoE therapy.

As used herein, the term “Cholangiocyte proliferation” refers to multiplication or reproduction of cholangiocytes, resulting in the expansion of the cholangiocyte population. A cholangiocyte is an epithelial cell that is part of the bile duct. Cholangiocytes contribute to bile secretion via net release of bicarbonate and water. Cholangiocyte proliferation is triggered during extrahepatic bile duct obstruction. The proliferative response of cholangiocytes during cholestasis is regulated by the complex interaction of several factors, including gastrointestinal hormones, neuroendocrine hormones and autocrine or paracrine signaling mechanisms.

As used herein, the term “Fibrosis” refers to the development of fibrous connective tissue as a reparative response to injury or damage. Fibrosis may refer to the connective tissue deposition that occurs as part of normal healing or to the excess tissue deposition that occurs as a pathological process.

As used herein, the term “Biliary obstruction” refers to the blockage of any duct that carries bile from the liver to the gallbladder or from the gallbladder to the small intestine. It can be caused by one or more of inflamed lymph nodes, Gallstones, Cysts, injury due to gallbladder surgery, tumors that have spread to the gallbladder, liver, pancreas, or bile ducts, inflamed bile ducts or liver damage

As used herein, the term “Hepatic fibrosis” refers to the wound healing response of the liver after acute or chronic injury which often results in the formation of scar tissue.

As used herein, the term “Liver abscess” refers to a pus-filled mass in the liver that can develop from injury to the liver or an intraabdominal infection disseminated from the portal circulation.

As used herein, the term “Liver cirrhosis” refers to a late-stage liver disease in which healthy liver tissue is replaced with scar tissue and the liver is permanently damaged.

As used herein, the term “Colitis” refers to inflammation of the large intestine or colon. Common symptoms include abdominal pain and bloating, bloody stools, Constant urge to have a bowel movement (tenesmus), dehydration and diarrhea.

As used herein, the term “Neonatal jaundice” refers to a liver condition that causes yellowing of a newborn baby's skin and eyes. Neonatal jaundice is common in preterm babies. The cause is often an immature liver. Infection, medications, or blood disorders.

As used herein, the term “Obstructive jaundice” refers to a specific type of jaundice, where symptoms develop due to a narrowed or blocked bile duct or pancreatic duct, preventing the normal drainage of bile from the bloodstream into the intestines.

As used herein, the term “Primary bile acid malabsorption” refers to an idiopathic intestinal disorder associated with congenital diarrhea, steatorrhea, interruption of the enterohepatic circulation of bile acids, and reduced plasma cholesterol levels.

As used herein, the term “Acute cholecystitis” refers to inflammation of the gallbladder that occurs due to occlusion of the cystic duct or impaired emptying of the gallbladder

As used herein, the term “Autoimmune hepatitis” refers to inflammation of the gallbladder that occurs due to occlusion of the cystic duct or impaired emptying of the gallbladder.

As used herein, the term “Benign recurrent intrahepatic cholestasis” (BRIC) is a rare genetic disorder characterized by intermittent episodes of jaundice and pruritus during which the liver cells have a reduced ability to secrete bile.

As used herein, the term “Cholestatic liver disease” refers to a group of diseases characterized by jaundice and cholestasis as the main presentation with different complications, which have considerable impact on the liver and can lead to end-stage liver disease, cirrhosis, and liver-related complications

As used herein, the term “Crigler-najjar syndrome” refers to rare autosomal recessive inherited disorder characterized by the absence or decreased activity of UDP-glucuronosyltransferase, an enzyme required for glucuronidation of unconjugated bilirubin in the liver. It is one of the major causes of congenital non-hemolytic jaundice.

As used herein, the term “Drug-induced hepatitis” refers to an acute or chronic liver injury secondary to drugs or herbal compounds. It can be classified based on clinical presentation (hepatocellular, cholestatic, or mixed), mechanism of hepatotoxicity, or histological appearance from a liver biopsy

As used herein, the term “Gilbert's syndrome” refers to an inherited (genetic) liver disorder that affects the body's ability to process bilirubin. Bilirubin is yellow liquid waste that occurs naturally as the body breaks down old red blood cells. Patients with Gilbert's syndrome don't produce enough liver enzymes to keep bilirubin at a normal level. As a result, excess bilirubin builds up in the body. Excess bilirubin is known as hyperbilirubinemia

As used herein, the term “Intrahepatic cholestasis of pregnancy” refers to a liver disorder in the late second and early third trimester of pregnancy. It is also known as obstetric cholestasis (OC) and is characterized by pruritus with increased serum bile acids and other liver function tests.

As used herein, the term “Liver rejection” refers to physiological reaction of the body to a foreign object. When a new liver is placed in a person's body, the body sees the transplanted organ as a threat and tries to attack it. The immune system makes antibodies to try to kill the new organ, not realizing that the transplanted liver is beneficial. Often to allow the organ to successfully live in a new body, medications are given to trick the immune system into accepting the transplant.

As used herein, the term “Non-alcoholic fatty liver disease” (NAFLD) refers to an umbrella term for a range of liver conditions affecting people who drink little to no alcohol. The main characteristic of NAFLD is too much fat stored in liver cells.

As used herein, the term “Non-alcoholic steatohepatitis” (NASH) refers to a type of liver disease in which fat builds up in the liver of people who drink little or no alcohol. This causes inflammation of the liver and damage to the cells in the liver, which may lead to cirrhosis (scarring of the liver) and liver failure. People with nonalcoholic steatohepatitis are at increased risk of developing liver cancer. It is more common in middle-aged adults, especially in those who are overweight or obese or who have diabetes or high levels of cholesterol and triglycerides (a type of fat) in the blood

As used herein, the term “Polycystic liver disease” refers to a rare condition that causes cysts or fluid-filled sacs to grow throughout the liver. The main symptom of polycystic liver disease is abdominal distension due to the large mass of cysts in the liver. Other symptoms that are also caused by the large bulk of cysts within the abdomen include shortness of breath, early postprandial fullness, abdominal discomfort, and back discomfort.

As used herein, the term “Veno-occlusive disease” refers to a condition in which some of the veins in the liver are blocked. This causes a decrease in blood flow inside the liver and may lead to liver damage. Signs and symptoms include weight gain, yellowing of the skin and whites of the eyes, dark-colored urine, and increased liver size. It may occur at some point in time after radiation therapy to the liver and bile ducts or after high-dose anticancer drugs were given before a stem cell transplant. Also called sinusoidal obstruction syndrome.

As used herein, the term “Wilson's disease” refers to an inherited disorder in which excessive amounts of copper accumulate in the body, particularly in the liver, brain, and eyes. The signs and symptoms of Wilson disease usually first appear between the ages of 6 and 45, but they most often begin during the teenage years. The features of this condition include a combination of liver disease and neurological and psychiatric problems.

As used herein the term “Eosinophilic gastrointestinal disorders (EGIDs)” refers to a group of rare gastrointestinal (GI) diseases which include Eosinophilic Gastritis (EoG), Eosinophilic Enteritis (EoN) and Eosinophilic Colitis (EoC). All of which are characterized by the presence of high amounts of eosinophils in the stomach or small intestine or large intestine. They affect different parts of the digestive tract, from the esophagus to the large intestine. Common symptoms include Heartburn, vomiting, stomach pain, weight loss, slow growth in children and diarrhea.

As used herein, the term “Eosinophilic gastritis and/or eosinophilic duodenitis (EG/EoD)” refers to a condition that causes inflammation throughout the digestive system. The symptoms associated with EG/EoD will vary from person to person, but can include vomiting, abdominal pain, nausea, bloating, constipation, diarrhea, difficulty in swallowing (dysphagia), ascites (fluid in the abdomen), heartburn and bloating.

As used herein, the term “Eosinophilic esophagitis (EoE)” refers to a chronic and progressive condition in which immune cells (eosinophils) build up within the esophagus and ends up damaging the esophagus. Common symptoms include difficulty swallowing (dysphagia), food getting stuck in the esophagus (impaction), choking, vomiting, chest pain/heartburn even after taking proton pump inhibitors (PPIs).

### III. Methods of Treatment

In embodiments, the disclosure encompasses methods for treating or preventing a hepatobiliary disease in a subject in need thereof comprising administering to the subject a therapeutic effective amount of compound of formula (I), or a pharmaceutically acceptable salt or solvate thereof.

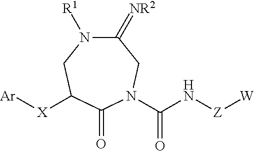

wherein,
Ar is (1) a C6 to C14 aromatic hydrocarbon group, (2) a 5 to 8-membered aromatic heterocyclic group having 1 to 4 hetero atoms selected from the group consisting of a nitrogen atom, sulfur atom, and oxygen atom, in place of a carbon atom, or (3) a bicyclic or tricyclic aromatic group formed by condensation of the above aromatic heterocyclic group and a C6 to C14 aromatic hydrocarbon ring,
where the above groups (1) to (3) of Ar are optionally substituted with 1 to 5 groups selected from the group consisting of:
(i) a halogen atom,
(ii) nitro,
(iii) cyano,
(iv) C1 to C6 alkyl optionally substituted with 1 to 3 halogen atoms,
(v) C2 to C6 alkenyl optionally substituted with 1 to 3 halogen atoms,
(vi) C2 to C6 alkynyl optionally substituted with 1 to 3 halogen atoms,
(vii) C3 to C6 cycloalkyl,
(xviii)hydroxyl,
(ix) C1 to C6 alkoxy optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(x) C1 to C5 alkylenedioxy,
(xi) C1 to C6 alkylthio optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(xii) amino,
(xiii) mono-C1 to C6 alkylamino,
(xiv) di-C1 to C6 alkylamino,
(xv) 5- to 6-membered cyclic amino,
(xvi) C1 to C6 alkylcarbonyl,
(xvii) carboxyl,
(xviii) C1 to C6 alkoxycarbonyl,
(xix) carbamoyl,
(xx) thiocarbamoyl,
(xxi) mono-C1 to C6 alkylcarbamoyl,
(xxii) di-C1 to C6 alkylcarbamoyl,
(xxii) 5- to 6-membered cyclic aminocarbonyl,
(xxiv) sulfo,
(xxv) C1 to C6 alkylsulfonyl
(xxvi) C1 to C6 alkoxycarbonylamino,
(xxvii) C1 to C6 alkylcarbonylamino,
(xxviii) mono- or di-C1 to C6 alkylaminocarbonylamino,
(xxix) aminosulfonyl, and
(xxx) mono- or di-C1 to C6 alkylaminosulfonyl, or substituted with 1 to 9 deuterium atoms,
X is (1) a connecting bond, (2) linear or branched C1 to C6 alkylene optionally substituted with 1 to 12 deuterium atoms, (3) an oxygen atom, (4) NR³, where R³ is a hydrogen atom or a C1 to C6 alkyl group, or (5) —S(O)m-, where m is an integer of 0 to 2,
Z is (1) a connecting bond or (2) CR⁴R⁵ where R⁴ and R⁵ are, independently,
(A) a hydrogen atom,
(B) a deuterium atom,
(C) C1 to C6 alkyl optionally substituted with 1 to 5 groups selected from the group consisting of (i) carboxyl, (ii) C1 to C6 alkoxycarbonyl, (iii) phenyl, (iv) hydroxyl, (v) C1 to C6 alkoxy, (vi) halogen atom, and (vii) C3 to C6 cycloalkyl, or substituted with 1 to 13 deuterium atoms,
(D) C3 to C6 cycloalkyl optionally substituted with 1 to 5 groups selected from the group consisting of (i) a halogen atom and (ii) an alkyl group optionally substituted with 1 to 3 halogen atoms or optionally substituted, with 1 to 11 deuterium atoms,
(E) COOR⁶, where R⁶ is a hydrogen atom or C1 to C6 alkyl, or
(F) CONR⁷R⁸, where R⁷ and R⁸ are, independently,
(a) hydrogen atom,
(b) C1 to C6 alkyl optionally substituted with 1 to 3 groups selected from the group consisting of (i) a halogen atom, (ii) C3 to C6 cycloalkyl, (iii) carboxyl, (iv) C1 to C6 alkoxycarbonyl, (v) C1 to C6 alkylcarbonyl, (vi) carbamoyl, (vii) mono-C1 to C6 alkylcarbamoyl, (viii) di-C1 to C6 alkylcarbamoyl, (ix) C6 to C12 aryl, and (x) C1 to C10 heteroaryl,
(c) C6 to C14 aromatic hydrocarbon group,
(d) a 5- to 8-membered aromatic heterocyclic group having 1 to 4 hetero atoms selected from the group consisting of a nitrogen atom, sulfur atom, and oxygen atom, in place of a carbon atom, or
(e) a bicyclic or tricyclic aromatic group formed by condensation of the above aromatic heterocyclic, group and a C6 to C14 aromatic hydrocarbon ring,
where each of the groups (c) to (e) is optionally substituted with 1 to 5 groups selected from the group consisting of (i) a halogen atom, (ii) nitro, (iii) cyano, (iv) C1 to C6 alkyl optionally substituted with 1 to 3 halogen atoms, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 halogen atoms, (x) C1 to C5 alkylenedioxy, (xi) C1 to C6 alkylthio optionally substituted with 1 to 3 halogen atoms, (xii) amino, (xiii) mono-C1 to C6 alkylamino, (xiv) di-C1 to C6 alkylamino, (xv) 5- to 6-membered cyclic amino, (xvi) C1 to C6 alkylcarbonyl, (xvii) carboxyl, (xviii) C1 to C6 alkoxycarbonyl, (xix) carbamoyl, (xx) thiocarbamoyl, (xxi) mono-C1 to C6 alkylcarbamoyl, (xxii) di-C1 to C6 alkylcarbamoyl, (xxiii) C6 to C10 arylcarbamoyl, (xxiv) C1 to C10 heteroarylcarbamoyl, (xxv) sulfo, (xxvi) C1 to C6 alkylsulfonyl, (xxvii) aminosulfonyl, and (xxviii) mono- or di-C1 to C6 alkylaminosulfonylamino, or substituted with 1 to 9 deuterium atoms,
W is (1) a hydrogen atom, (2) a C6 to C14 aromatic hydrocarbon group, (3) a 5- to 8-membered aromatic heterocyclic group having 1 to 4 hetero atoms selected from a nitrogen atom, a sulfur atom, and an oxygen atom, in place of a carbon atom, (4) a bicyclic, or tricyclic aromatic group formed by condensation of the above aromatic heterocyclic group and a C6 to C14 aromatic hydrocarbon ring, or a deuterium atom,
where, each of the groups (2) to (4) of the above W is optionally substituted with 1 to 5 groups selected from the group consisting of (i) a halogen atom, (ii) nitro, (iii) cyano, (iv) C1 to C6 alkyl optionally substituted with 1 to 3 groups selected from the group consisting of a halogen atom, amino, C1 to C6 alkoxycarbonyl, C1 to C6 alkoxycarbonylamino, and carboxyl, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 groups selected from the group consisting of a halogen atom, hydroxyl, C1 to C6 alkoxy, amino and mono- or di-C1 to C6 alkylamino, (x) C1 to C5 alkylenedioxy, (xi) C1 to C6 alkylthio optionally substituted with 1 to 3 groups selected from the group consisting of a halogen atom, hydroxyl, C1 to C6 alkoxy, amino, and mono- or di-C1 to C6 alkylamino, (xii) amino, (xiii) mono-C1 to C6 alkylamino, (xiv) di-C1 to C6 alkylamino, (xv) 5- to 6-membered cyclic amino, (xvi) C1 to C6 alkyl-carbonyl, (xvii) carboxyl, (xviii) C1 to C6 alkoxycarbonyl optionally substituted with a halogen atom, (xix) C7 to C16 aralkyloxycarbonyl optionally substituted with a halogen atom, (xx) carbamoyl, (xxi) mono-C1 to C6 alkylcarbamoyl optionally substituted with 1 to 3 groups which are selected from the group consisting of a halogen atom, hydroxyl, carboxyl, C1 to C6 alkoxy, amino, and mono- or di-C1 to C6 alkylamino, (xxii) di-C1 to C6 alkylcarbamoyl optionally substituted with hydroxyl, (xxiii) 5- to 6-membered cyclic aminocarbonyl optionally substituted with C1 to C6 alkoxycarbonyl, (xxiv) C6 to C10 arylcarbamoyl, (xxv) C1 to C10 heteroarylcarbamoyl, (xxvi) C7 to C16 aralkylcarbamoyl, (xxvii) C1 to C10 heteroaryl-C1 to C6 alkylcarbamoyl, (xxviii) N C1 to C6 alkyl-N—C6 to C12 arylcarbamoyl, (xxix) C3 to C6 cycloalkylcarbamoyl, (xxx) sulfo, (xxxi) C1 to C6 alkylsulfonyl, (xxxii) C1 to C6 alkylsulfonylamino, (xxxiii) C6 to C12 arylsulfonylamino optionally substituted with C1 to C6 alkyl, (xxxiv) C1 to C10 heteroarylsulfonylamino, (xxxv) C1 to C6 alkoxycarbonylamino, (xxxvi) C1 to C6 alkylcarbonylamino, (xxxvii) mono- or di-C1 to C6 alkylaminocarbonylamino, (xxxviii) C6 to C12 aryl, (xxxix) C1 to C10 heteroaryl, (xl) C6 to C10 aryloxy, (xli) C1 to C10 heteroaryloxy, (xlii) C7 to C16 aralkyloxy, (xliii) C1 to C10 heteroaryl-C1 to C6 alkyloxy, (xliv) aminosulfonyl, (xlv) mono- or di-C1 to C6 alkylaminosulfonyl, (xlvi) C7 to C16 aralkyloxycarbamoyl, and (xlvii) C1 to C10 heteroaryl-C1 to C6 alkyloxycarbamoyl, or substituted with 1 to 9 deuterium atoms,
R1 is
(1) a hydrogen atom,
(2) C1 to C6 alkyl optionally substituted with 1 to 3 of (i) a halogen atom or (ii) C3 to C6 cycloalkyl or substituted with (iii) 1 to 13 deuterium atoms,
(3) C2 to C6 alkenyl optionally substituted with 1 to 3 of (i) a halogen atom or (ii) C3 to C6 cycloalkyl or substituted with (iii) 1 to 11 deuterium atoms,
(4) C2 to C6 alkynyl optionally substituted with 1 to 3 of (i) a halogen atom or (ii) C3 to C6 cycloalkyl, or substituted with (iii) 1 to 9 deuterium atoms, or
(5) C3 to C6 cycloalkyl optionally substituted with 1 to 3 of (i) a halogen atom or (ii) C3 to C6 cycloalkyl, or substituted with (iii) 1 to 11 deuterium atoms,
R2 is (1) OR⁹ or (2) NR¹⁰R¹¹
where, R⁹, R¹⁰, and R¹¹ independently indicate
(A) a hydrogen atom,
(B) C1 to C6 alkyl,
(C) C2 to C6 alkenyl,
(D) C3 to C6 alkynyl,
(E) C3 to C6 cycloalkyl
(F) C6 to C14 aromatic hydrocarbon group,
(G) a 5- to 8-membered aromatic heterocyclic group having 1 to 4 hetero atoms selected from the group consisting of a nitrogen atom, sulfur atom, and oxygen atom, in place of a carbon atom, or
(H) a bicyclic or tricyclic aromatic group formed by condensation of the above aromatic heterocyclic group and a C6 to C14 aromatic hydrocarbon ring,
where each of the groups of the above (B) to (E) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5 to 6-membered cyclic amino, (ix) carboxyl, (x) C1 to C6 alkoxycarbonyl, (xi) C1 to C6 alkylcarbonyl, (xii) carbamoyl, (xiii) mono-C1 to C6 alkylcarbamoyl, (xiv) di-C1 to C6 alkylcarbamoyl, (xv) C6 to C12 aryl, and (xvi) C1 to C10 heteroaryl, or substituted with 1 to 13 deuterium atoms, further, each of the groups of the above (F) to (H) are optionally substituted with 1 to 5 groups which are selected from the group consisting of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms, or R1 and R2 may form 5- or 6-member heterocyclic ring together with the atoms they are bonded with, where the above 5- or 6-member heterocyclic ring is optionally substituted with 1 to 3 groups selected from the group consisting of
(A) a halogen atom,
(B) oxo,
(C) hydroxyl
(D) C1 to C6 alkyl,
(E) C2 to C6 alkenyl,
(F) C2 to C6 alkynyl,
(G) C3 to C6 cycloalkyl
(H) C6 to C14 aromatic hydrocarbon group,
(I) 5- to 8-membered aromatic heterocyclic group having 1 to 4 hetero atoms selected from the group consisting of a nitrogen atom, sulfur atom, and oxygen atom, in place of a carbon atom, and
(J) a bicyclic or tricyclic aromatic group formed by condensation of the above aromatic heterocyclic group and a C6 to C14 aromatic hydrocarbon ring, or substituted with
(K) 1 to 6 deuterium atoms,
where each of the groups of the above (D) to (G) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5- to 6-membered cyclic amino, (ix) carboxyl, (x) C1 to C6 alkoxycarbonyl, (xi) C1 to C6 alkyl-carbonyl, (xii) carbamoyl, (xiii) mono-C1 to C6 alkylcarbamoyl, (xiv) di-C1 to C6 alkylcarbamoyl, (xv) C6 to C12 aryl and (xvi) C1 to C10 heteroaryl, or substituted with 1 to 13 deuterium atoms, each of the groups of the above (H) to (J) is optionally substituted with 1 to 5 groups which are selected from the group consisting, of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms.

In an embodiment, the compound of Formula (I) or a pharmaceutically acceptable salt or solvate thereof is selected from the group consisting of:
(1) 4-{(1R)-1-[({(6R)-6-(5-chloro-2-methoxybenzyl)-7-oxo-3-[(pyridin-2-yloxy)imino]-1,4-diazepan-1-yl}carbonyl)amino]propyl}benzoic acid,
(2) 2-amino-4-[(1R)-1-({[(6S)-6-(5-chloro-2-methoxybenzyl)-3-(ethoxyimino)-7-oxo-1,4-diazepan-1-yl]carbonyl}amino)butyl]benzoic acid,
(3) 2-amino-4-{(1R)-1-[({(6S)-6-(5-chloro-2-methoxybenzyl)-7-oxo-3-[(2,2,2-trifluoroethoxy)imino]-1,4-diazepan-1-yl}carbonyl)amino]butyl}benzoic acid,
(4) 2-amino-4-{(1R)-1-[({(6R)-6-(5-chloro-2-methoxybenzyl)-3-[(3,5-difluorophenoxy)imino]-7-oxo-1,4-diazepan-1-yl}carbonyl)amino]ethyl}benzoic acid,
(5) 2-amino-4-[(1R)-1-({[(6S)-6-(5-chloro-2-methoxybenzyl)-3-(methoxyimino)-4-methyl-7-oxo-1,4-diazepan-1-yl]carbonyl}amino)butyl]benzoic acid,
(6) (4-{(1R)-1-[({(6R)-6-(5-chloro-2-methoxybenzyl)-3-[(3,5-difluorophenoxy)imino]-7-oxo-1,4-diazepan-1-yl}carbonyl)amino]ethyl}phenyl) acetic acid,
(7) 2-amino-4-{[({(6R)-6-(5-chloro-2-methoxybenzyl)-3-[(3,5-difluorophenoxy)imino]-7-oxo-1,4-diazepan-1-yl}carbonyl)amino]methyl}benzoic acid,
(8) {4-[(1R)-1-({[(6R)-6-(5-chloro-2-methoxybenzyl)-7-oxo-3-(phenoxyimino)-1,4-diazepan-1-yl]carbonyl}amino)propyl]phenyl}acetic acid,
(9) 2-amino-4-[(1R)-1-({[(6R)-6-(5-chloro-2-methoxybenzyl)-7-oxo-3-(phenoxyimino)-1,4-diazepan-1-yl]carbonyl}amino)propyl]benzoic acid, or
(10) 2-amino-4-[(1R)-1({[(6R)-6-(5-chloro-2-methoxybenzyl)-3-(ethoxyimino)-7-oxo-1,4-diazepan-1-yl]carbonyl}amino)butyl]benzoic acid.

In embodiments, the compound of Formula (I) is highly selective chymase inhibitor over cathepsin G. Due to high selectivity, compound of formula (I) has lesser side effects as compared to the non-selective chymase inhibitors after prolonged use in treating hepatobiliary disease

In embodiments, the compound of Formula (I) is 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid (compound 1):

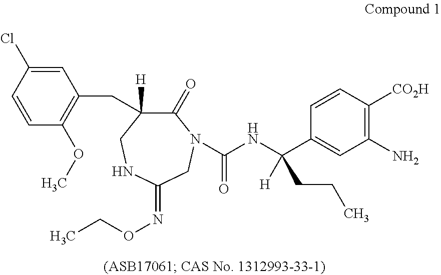 [SMILES: CCC[C@@H](NC(=O)N1C/C(=N/OCC)NC[C@H](Cc2cc(Cl)ccc2OC)C1=O)c1ccc(C(=O)O)c(N)c1]

or a pharmaceutically acceptable salt or solvate thereof.

In preferred embodiment, the compound 1 is monoacetic acid solvate also known as compound 1 monoacetic acid solvate:

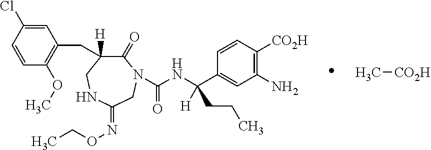

In embodiments, Compound 1 monoacetic acid solvate is approx. 1600 folds selective for chymase over cathepsin G. In embodiments, Compound 1 monoacetic acid solvate has lesser side effects than other non-selective chymase inhibitors in treating hepatobiliary disease.

In embodiments, hepatobiliary disease is selected from the group consisting of acute hepatitis, alcoholic hepatitis, alcoholic liver disease, extrahepatic cholestasis, hepatic fibrosis, liver abscess, liver cirrhosis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, nonalcoholic fatty liver disease, non-alcoholic steatohepatitis, polycystic liver disease, PBC, PSC, veno-occlusive disease, Wilson's disease. In embodiments, hepatobiliary disease includes PSC. In embodiments, hepatobiliary disease includes PBC.

In embodiments, the disclosure encompasses a method of treating hepatobiliary disease in a subject in need thereof, wherein the administration of compound of Formula (I) or a pharmaceutically acceptable salt or solvate thereof ameliorates jaundice.

In embodiments, the disclosure encompasses a method of treating hepatobiliary disease in a subject in need thereof, wherein the administration of compound of Formula (I) or a pharmaceutically acceptable salt or a solvate thereof ameliorates pruritis.

In embodiments, the disclosure encompasses a method of treating hepatobiliary disease in a subject in need thereof, wherein the administration of compound of Formula (I) or a pharmaceutically acceptable salt or a solvate thereof ameliorates inflammation.

In various embodiments, the disclosure encompasses a treatment regimen, which involves administration of pharmaceutical formulation to a patient in need thereof once a day, twice a day, thrice a day, once in two days, or once in three days or once a week. The administration is preferably oral, but other routes are also contemplated, including for example, rectal administration, or intracolonic administration.

In embodiments, the pharmaceutical formulation comprises one or more second therapeutic agents.

In certain embodiments, the method comprises administering one or more second formulations comprising one or more second therapeutic agents to the subject as an adjuvant therapy. In various embodiments, the second formulation may be administered either simultaneously or sequentially separated by a specific period of time. In embodiments, the second formulation may be administered by the same route of administration as the first formulation or a different route of administration as the first formulation. In embodiments, the second formulation is an immediate-release formulation, extended-release formulation or delayed-release formulation.

In embodiments, the one or more second therapeutic agents is steroids (for example, betamethasone etc.), immunosuppressants (for example, tacrolimus and pimecrolimus etc.), antiallergic agent (clemastine fumarate, d-chlorpheniramine maleate, cyproheptadine hydrochloride, promethazine hydrochloride, homochlorcyclizine hydrochloride, mequitazine, diphenhydramine hydrochloride, ebastine, cetirizine hydrochloride, olopatadine hydrochloride, fexofenadine hydrochloride, sodium cromoglicate, emedastine fumarate, suplatast tosilate and epinastine hydrochloride, etc.

In embodiments, the disclosure encompasses a method for treating or preventing hepatobiliary disease in a subject, comprising administering to the subject a therapeutic effective amount of compound 1 or a pharmaceutically acceptable salt or solvate thereof.

In embodiments, the disclosure encompasses a method for treating or preventing jaundice in a subject suffering with hepatobiliary disease in a subject, comprising administering to the subject a therapeutic effective amount of compound 1 or its pharmaceutically acceptable salt or solvate thereof.

In embodiments, the disclosure encompasses a method for treating or preventing one or more of hepatomegaly, cirrhosis, biliary obstruction, occult blood in stool, fecal incontinence, fever, fatigue, weight loss abdominal pain, diarrhea, nausea, spasm, mast cell and eosinophil counts, dysphagia, gastroesophageal reflux disease (GERD), bloating, early satiety, loss of appetite, and pruritis in a subject suffering with hepatobiliary disease in a subject, comprising administering to the subject a therapeutic effective amount of compound 1 or its pharmaceutically acceptable salt or solvate thereof.

In embodiments, the disclosure encompasses a method for treating or preventing inflammation in a subject suffering with hepatobiliary disease, comprising administering to the subject a therapeutic effective amount of compound 1 or its pharmaceutically acceptable salt or solvate thereof.

In embodiments, the disclosure encompasses a method for reducing mast cell infiltration in a subject suffering with hepatobiliary disease, comprising administering to the subject a therapeutic effective amount of compound 1 or its pharmaceutically acceptable salt or solvate thereof.

In embodiments, the disclosure encompasses a method of treating or preventing hepatobiliary disease in a subject comprising administering to said subject therapeutic effective amount of Compound 1 or a pharmaceutically acceptable salt or solvate thereof in an amount from about 10 mg to about 1000 mg per unit dose, e.g., about 50 mg to about 950 mg per unit dose, about 10 mg to about 900 mg per unit dose, about 10 mg to about 850 mg per unit dose, about 10 mg to about 800 mg per unit dose, about 10 mg to about 750 mg per unit dose, about 10 mg to about 700 mg per unit dose, about 10 mg to about 650 mg per unit dose, about 10 mg to about 600 mg per unit dose, about 50 mg to about 550 mg per unit dose, about 50 mg to about 500 mg per unit dose, about 50 mg to about 450 mg per unit dose, about 50 mg to about 400 mg per unit dose, about 50 mg to about 350 mg per unit dose, about 50 mg to about 300 mg per unit dose, about 50 mg to about 250 mg per unit dose, about 50 mg to about 200 mg per unit dose, about 50 mg to about 150 mg per unit dose, about 50 mg to about 100 mg per unit dose.

In embodiments, hepatobiliary disease is selected from the group consisting of acute hepatitis, alcoholic hepatitis, alcoholic liver disease, extrahepatic cholestasis, hepatic fibrosis, liver abscess, liver cirrhosis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, nonalcoholic fatty liver disease, non-alcoholic steatohepatitis, polycystic liver disease, PBC, PSC, veno-occlusive disease, Wilson's disease. In embodiments, hepatobiliary disease includes PSC. In embodiments, hepatobiliary disease includes PBC.

In embodiments, the disclosure encompasses a method for treating or preventing PSC in a subject in need thereof, said method comprising administering to said subject a therapeutic effective amount of compound 1 monoacetic acid solvate. In embodiments, compound 1 monoacetic acid solvate is in the range of about 10 mg-100 mg per unit dose

In embodiments, the disclosure encompasses a method for treating or preventing PBC in a subject in need thereof, said method comprising administering to said subject a therapeutic effective amount of compound 1 monoacetic acid solvate. In embodiments, compound 1 monoacetic acid solvate is in the range of about 10 mg-100 mg per unit dose

In embodiments, the disclosure encompasses a method for treating or preventing EGID in a subject in need thereof, said method comprising administering to said subject a therapeutic effective amount of a compound according to Formula (I) or a pharmaceutically acceptable salt thereof or a solvate thereof.

In embodiments, the disclosure encompasses a method for treating or preventing colitis in a subject in need thereof, said method comprising administering to said subject a therapeutically effective amount of the compounds of the present invention, including compound 1 monoacetic acid solvate. In embodiments, such compounds, including compound 1 monoacetic acid solvate, are in the range of about 10 mg-100 mg per unit dose. In embodiments, the disclosure further encompasses a method for treating or preventing Ulcerative colitis (UC) in a subject in need thereof, said method comprising administering to said subject a therapeutic effective amount of the compounds of the present invention, including compound 1 monoacetic acid solvate. In embodiments, such compounds, including compound 1 monoacetic acid solvate are in the range of about 10 mg-100 mg per unit dose.

In embodiments, the disclosure encompasses a method for treating or preventing Crohn's disease (CD) in a subject in need thereof, said method comprising administering to said subject a therapeutic effective amount of the compounds of the present invention, including compound 1 monoacetic acid solvate. In embodiments, such compounds, including compound 1 monoacetic acid solvate are in the range of about 10 mg-100 mg per unit dose

In embodiments, the disclosure encompasses a method for treating or preventing EGID in a subject in need thereof, said method comprising administering to said subject a therapeutic effective amount of a compound 1 or a pharmaceutically acceptable salt thereof or a solvate thereof.

In certain embodiments, EGID is selected from the group consisting of eosinophilic gastroenteritis, eosinophilic esophagitis, eosinophilic duodenitis, eosinophilic gastritis.

### IV. Synthesis of Compounds

The compounds of the present disclosure can be readily synthesized by a variety of methods known in the art. The synthesis of seven membered ring compounds of the present disclosure is disclosed in U.S. Pat. No. 8,846,660, which is hereby incorporated by reference in its entirety and relied upon.

### V. Dosage and Administration

In various embodiments, the compound of Formula (I) or a pharmaceutically acceptable salt or solvate thereof is administered at a dose ranging from, about 10 mg to 1000 mg per unit dose (about 50 mg to about 950 mg per unit dose, about 10 mg to about 900 mg per unit dose, about 10 mg to about 850 mg per unit dose, about 10 mg to about 800 mg per unit dose, about 10 mg to about 750 mg per unit dose, about 10 mg to about 700 mg per unit dose, about 10 mg to about 650 mg per unit dose, about 10 mg to about 600 mg per unit dose, about 50 mg to about 550 mg per unit dose, about 50 mg to about 500 mg per unit dose, about 50 mg to about 450 mg per unit dose, about 50 mg to about 400 mg per unit dose, about 50 mg to about 350 mg per unit dose, about 50 mg to about 300 mg per unit dose, about 50 mg to about 250 mg per unit dose, about 50 mg to about 200 mg per unit dose, about 50 mg to about 150 mg per unit dose, about 50 mg to about 100 mg per unit dose). In some embodiments, compound of formula (I) (in milligram per kg body weight of the subject) is in the range of from about 0.1 mg/kg to about 100 mg/kg.

In certain embodiments, the dose of compound of Formula (I) or a pharmaceutically acceptable salt thereof (in milligrams per kg body weight of the subject) is in the range of from about 0.1 to about 100 mg/kg, preferably about 1 to 50 mg/kg, more preferably about 1 to about 10 mg/kg, to be administered once a day, twice a day, thrice a day, once in two days, or once in three days or once a week. In some embodiments, the unit dose of Compound 1 or a pharmaceutically acceptable salt thereof (total unit dose) is in the range of from about 10 mg to about 1000 mg per unit dose, e.g., about 50 mg to about 500 mg.

In certain embodiments, the dose of compound 1, or a pharmaceutically acceptable salt or solvate thereof (based on the amount of active ingredient) (in milligrams per kg body weight of the subject) is about 0.1 mg/kg to about 100 mg/kg, e.g., about 0.2 mg/kg, about 0.4 mg/kg, about 0.6 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.8 mg/kg, about 3.0 mg/kg, about 3.2 mg/kg, about 3.4 mg/kg, about 3.6 mg/kg, about 3.8 mg/kg, about 4.0 mg/kg, about 4.2 mg/kg, about 4.4 mg/kg, about 4.6 mg/kg, about 4.8 mg/kg, about 5.0 mg/kg, about 5.2 mg/kg, about 5.4 mg/kg, about 5.6 mg/kg, about 5.8 mg/kg, about 6.0 mg/kg, about 6.2 mg/kg, about 6.4 mg/kg, about 6.6 mg/kg, about 6.8 mg/kg, about 7.0 mg/kg, about 7.2 mg/kg, about 7.4 mg/kg, about 7.6 mg/kg, about 7.8 mg/kg, about 8.0 mg/kg, about 8.2 mg/kg, about 8.4 mg/kg, about 8.6 mg/kg, about 8.8 mg/kg, about 9.0 mg/kg, about 9.2 mg/kg, about 9.4 mg/kg, about 9.6 mg/kg, about 9.8 mg/kg, or about 10.0 mg/kg, about 12 mg/kg, about 14 mg/kg, about 16 mg/kg, about 18 mg/kg, about 20 mg/kg, about 22 mg/kg, about 24 mg/kg, about 26 mg/kg, about 28 mg/kg, about 30 mg/kg, about 32 mg/kg, about 34 mg/kg, about 36 mg/kg, about 38 mg/kg, about 40 mg/kg, about 42 mg/kg, about 44 mg/kg, about 46 mg/kg, about 48 mg/kg, about 50 mg/kg, about 52 mg/kg, about 54 mg/kg, about 56 mg/kg, about 58 mg/kg, about 60 mg/kg, about 62 mg/kg, about 64 mg/kg, about 66 mg/kg, about 68 mg/kg, about 70 mg/kg, about 72 mg/kg, about 74 mg/kg, about 76 mg/kg, about 78 mg/kg, about 80 mg/kg, about 82 mg/kg, about 84 mg/kg, about 86 mg/kg, about 88 mg/kg, about 90 mg/kg, about 92 mg/kg, about 94 mg/kg, about 96 mg/kg, about 98 mg/kg, or about 100 mg/kg including all values and ranges in between.

The unit dose of compound 1 or a pharmaceutically acceptable salt or solvate thereof (total unit dose), is about 10 mg to 1000 mg, e.g., about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, including all values and ranges in between. In embodiments, the unit dose of compound 1 or a pharmaceutically acceptable salt or solvate thereof in a pharmaceutical formulation is administered, once a day, twice a day, thrice a day, once in two days, or once in three days or once a week. In embodiments, the unit dose of ondansetron is administered once daily. In embodiments, the unit dose of ondansetron is administered twice daily. In various embodiments, the pharmaceutical compositions, formulations, and dosage forms can be administered one or more times per day for a period of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days and can be extended for 1 or more month or administered at various intervals in the above time frame.

In various embodiments, a range of unit doses of compound 1 or a pharmaceutically acceptable salt or solvate thereof is used in the pharmaceutical formulation in the present disclosure includes about 10 mg to about 1000 mg, about 50 mg to about 950 mg, about 50 mg to about 900 mg, about 50 mg to about 850 mg, 50 mg to about 800 mg, about 50 mg to about 750 mg, about 50 mg to about 700 mg about 50 mg to about 650 mg, about 50 mg to about 600 mg, about 50 mg to about 550 mg, about 50 mg to about 500 mg, about 50 mg to about 450 mg, about 50 mg to about 400 mg, about 50 mg to about 350 mg, about 50 mg to about 300 mg about 50 mg to about 250 mg, about 50 mg to about 200 mg, about 50 mg to about 150 mg, about 50 mg to about 100 mg. In embodiments, the dose is administered once a day, twice a day, thrice a day, once in two days, or once in three days or once a week.

In various embodiments, the unit dose of compound 1 or its pharmaceutically acceptable salt or solvate thereof included in the present disclosure is about 10 mg, about 50 mg, about 100 mg, about 150 mg, about 200 mg, about 250 mg, about 300 mg, about 350 mg, about 400 mg about 450 mg, about 500 mg, about 550 mg, about 600 mg, about 650 mg, about 700 mg, about 750 mg, about 800 mg, about 900 mg, about 950 mg, about 1000 mg. The dose may be administered once a day, twice a day, thrice a day, once in two days, or once in three days or once a week.

In embodiments, the preferred dose of compound 1 or a pharmaceutically acceptable salt or solvate thereof is about 50 mg, about 55 mg, about 60 mg, about 65 mg, about 70 mg, about 75 mg, about 80 mg, about 85 mg, about 90 mg, about 95 mg or about 100 mg, about 150 mg. The dose may be administrated once a day, twice a day, thrice a day, once in two days, or once in three days or once a week.

### VI. Pharmaceutical Compositions

In embodiments, the present disclosure provides a pharmaceutical composition comprising compound of Formula (I) or a pharmaceutically acceptable salt or a solvate thereof for the treatment of hepatobiliary disease. In embodiments, the disease is selected from acute hepatitis, alcoholic hepatitis, alcoholic liver disease, colitis, extrahepatic cholestasis, hepatic fibrosis, liver abscess, liver cirrhosis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, non-alcoholic steatohepatitis, polycystic liver disease, PBC, PSC, veno-occlusive disease, Wilson's disease.

In another embodiment, the present disclosure provides a pharmaceutical composition comprising compound of Formula (I) or a pharmaceutically acceptable salt or a solvate thereof for the treatment of EGID. In embodiments, the EGID is selected from the group consisting of eosinophilic gastroenteritis and eosinophilic esophagitis, eosinophilic duodenitis, eosinophilic gastritis.

In an embodiment, compound of Formula (I) is compound 1 or pharmaceutically acceptable salt or solvate thereof. In embodiments, compound 1 or pharmaceutically acceptable salt or solvate thereof is 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate.

In embodiments, the present disclosure provides a pharmaceutical composition comprising a therapeutic effective amount of compound of Formula (I), or a pharmaceutically acceptable salt or a solvate thereof together with one or more acceptable adjuvants (e.g., carriers or excipients).

The pharmaceutical compositions of the present disclosure may be administered by any convenient route, which suits the desired therapy. The oral or parenteral route are suitable for administering pharmaceutical compositions to treat hepatobiliary disease (e.g., PSC, PBC).

The pharmaceutical composition of the present disclosure can also be administered orally with site specific or targeted delivery of the compound of formula (I) or compound 1 as active agent. In some embodiments, the compositions are designed for targeted delivery for example, gut targeted delivery, liver targeted delivery. In embodiments, the oral compositions may be immediate or modified or delayed or timed phase release. In some embodiments, the oral compositions for specific site delivery may include tablets, troches, lozenges, dispersible powders, pellets, multiunit particulate system (MUPS) granules, a hard or soft capsule, suspension, solution or the like. In some embodiments, the pharmaceutical formulations can be in unit dosage form.

Accordingly, the present disclosure also provides pharmaceutical compositions including a pharmaceutically acceptable carrier or excipient and either a conjugate of the present disclosure, or a pharmaceutically acceptable salt of a compound of the present disclosure. Appropriate excipients can be tailored to the particular composition and route of administration by methods well known in the art, e.g., REMINGTON'S PHARMACEUTICAL SCIENCES.

An oral pharmaceutical composition may further contain carrier, such as diluents, waxes, flavouring agents, binders, preservatives, tablet disintegrating agents, bioadhesive polymers, polymers or an encapsulating material or coating material (e.g., enteric coating material) or the like. In some embodiments, the polymer may be immediate release polymer, extended-release polymer, controlled release polymer, enteric coated polymer or colon release polymer or the like. Details on techniques for formulation and administration are well described in the scientific and patent literature, see, e.g., the latest edition of Remington's Pharmaceutical Sciences, Maack Publishing Co, Easton Pa. (“Remington's”).

In some embodiments, in solid compositions such as tablets the active agent is mixed with a pharmaceutical carrier. Pharmaceutical carrier which may be incorporated into the tablets of the present disclosure include one or more of the following: diluents, waxes, flavoring agents, binders, preservatives, tablet disintegrating agents, polymers or an encapsulating material or coating material (e.g., enteric coating material) or the like Conventional tableting ingredients include a binder such as starch, corn starch, polyvinylpyrrolidone, gums, gum acacia, gelatin, cellulose derivatives and combination thereof; a diluent such as Microcrystalline Cellulose, Lactose, Dibasic or Tribasic Calcium Phosphate, Saccharides Confectioner's Sugar, Compressible Sugar, Dextrates, Dextrin, Dextrose, Fructose, Lactitol, Mannitol, Sucrose, Starch Xylitol, Sorbitol, Talc, Calcium Carbonate, Calcium Sulphate and combination thereof; a lubricant such as Mg, Al or Ca or Zn Stearate, Polyethylene Glycol, Glyceryl Behenate, Glyceryl Monosterate, Mineral Oil Sodium Stearyl Fumarate, Stearic Acid, Hydrogenated Vegetable Oil, Talc, Hydrogenated Soybean Oil, Stearyl Alcohol and combination thereof; a glidant such as Magnesium Trisilicate, Powdered Cellulose, Starch, Talc, Tribasic Calcium Phosphate, Calcium Silicate, Magnesium Stearate, Magnesium Silicate, Colloidal Silicon Dioxide, or Silicon Hydrogel and combination thereof; a Surface active agent; a wax; a disintegrating agent; a bioadhesive polymer, which includes proteins such as Pectin, Zein, or the likes, Polysaccharides such as Cellulose, Dextrans, or the likes, Polyamides, Polycarbonates, Polyalkylenes, Polyalkylene Glycols, Polyalkylene Oxides, Polyalkylene Terephthalates, Polyvinyl Alcohols, Polyvinyl Ethers, Polyvinyl Esters, Polyvinyl Halides, Polyvinylpyrrolidone, Polyglycolides, Polysiloxanes, Polyurethanes, Polystyrene, Polymers of Acrylic And Methacrylic Esters, Polylactides, Polyanhydrides, Polyorthoesters and combination thereof; a Controlled release polymer may be but not limited to Ethyl Acrylate and Methyl Methacrylate copolymer dispersion individual and combination thereof; a modified release polymer such as Cellulose Derivatives Vinyl Pyrrolidone Polymers and copolymers; Alkylene Oxide and water. These can be used to form a solid preformulation containing a homogeneous mixture of a compound of the present disclosure, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form for site specific drug delivery, affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. In some embodiments the pharmaceutical formulation may be in the form of suspension

Additionally, pigments and for colorants may be added.

In some embodiments, pharmaceutical formulations include a drug targeting molecule conjugate of the present disclosure complexed with liposomes. Methods to produce liposomes are known to those of skill in the art.

In some embodiments, sustained-release preparations comprising drug targeting molecule conjugates described herein can be produced. Suitable examples of sustained-release preparations include semi-permeable matrices of solid hydrophobic polymers containing a drug conjugate, where the matrices are in the form of shaped articles (e.g., films or microcapsules). Methods to produce sustained-release preparation are known to those of skill in the art. In some embodiments, pharmaceutical composition suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include: water, ethanol, polyols (propyleneglycol, polyethyleneglycol, glycerol, and the like), suitable mixtures thereof, vegetable oils (such as olive oil), and injectable organic esters (such as ethyl oleate). Proper fluidity can be maintained, for example, by the use of a coating such as lecithin, by the maintenance of the required particle size in the case of dispersions, and by the use of surfactant.

### VII. Hepatobiliary Disease

In embodiments, the method of the present disclosure is useful for the treatment of various hepatobiliary diseases and hepatobiliary-related diseases. In embodiments, the disease is selected from the group consisting of acute hepatitis, alcoholic hepatitis, alcoholic liver disease, extrahepatic cholestasis, hepatic fibrosis, liver abscess, liver cirrhosis, neonatal jaundice, obstructive jaundice, primary bile acid malabsorption, acute cholecystitis, autoimmune hepatitis, benign recurrent intrahepatic cholestasis, colitis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, non-alcoholic steatohepatitis, polycystic liver disease, PBC, PSC, veno-occlusive disease, Wilson's disease. In embodiments, hepatobiliary disease is PSC. In embodiments, hepatobiliary disease is PBC.

In embodiments, the therapeutic effective amount of Compound of formula (I) or a pharmaceutically acceptable salt or solvate thereof is useful in treating acute hepatitis. In embodiments, the therapeutic effective amount of Compound of formula (I) or a pharmaceutically acceptable salt or solvate thereof is useful in treating alcoholic hepatitis. In embodiments, the therapeutic effective amount of Compound of formula (I) or a pharmaceutically acceptable salt or solvate thereof is useful in treating alcoholic liver disease. In embodiments, the therapeutic effective amount of Compound of formula (I) or a pharmaceutically acceptable salt or solvate thereof is useful in treating extrahepatic cholestasis. In embodiments, the therapeutic effective amount of Compound of formula (I) or a pharmaceutically acceptable salt or solvate thereof is useful in treating hepatic fibrosis. In embodiments, the therapeutic effective amount of Compound of formula (I) or a pharmaceutically acceptable salt or solvate thereof is useful in treating liver abscess. In embodiments, the therapeutic effective amount of Compound of formula (I) or a pharmaceutically acceptable salt or solvate thereof is useful in treating liver cirrhosis. In embodiments, the therapeutic effective amount of Compound of formula (I) or a pharmaceutically acceptable salt or solvate thereof is useful in treating neonatal jaundice. In embodiments, the therapeutic effective amount of Compound of formula (I) or a pharmaceutically acceptable salt or solvate thereof is useful in treating obstructive jaundice. In embodiments, the therapeutic effective amount of Compound of formula (I) or a pharmaceutically acceptable salt or solvate thereof is useful in treating primary bile acid malabsorption. In embodiments, the therapeutic effective amount of Compound of formula (I) or a pharmaceutically acceptable salt or solvate thereof is useful in treating acute cholecystitis. In embodiments, the therapeutic effective amount of Compound of formula (I) or a pharmaceutically acceptable salt or solvate thereof is useful in treating autoimmune hepatitis. In embodiments, the therapeutic effective amount of Compound of formula (I) or a pharmaceutically acceptable salt or solvate thereof is useful in treating benign recurrent intrahepatic cholestasis. In embodiments, the therapeutic effective amount of Compound of formula (I) or a pharmaceutically acceptable salt or solvate thereof is useful in treating biliary obstruction. In embodiments, the therapeutic effective amount of Compound of formula (I) or a pharmaceutically acceptable salt or solvate thereof is useful in treating cholestatic liver disease. In embodiments, the therapeutic effective amount of Compound of formula (I) or a pharmaceutically acceptable salt or solvate thereof is useful in treating crigler-najjar syndrome. In embodiments, the therapeutic effective amount of Compound of formula (I) or a pharmaceutically acceptable salt or solvate thereof is useful in treating drug-induced hepatitis. In embodiments, the therapeutic effective amount of Compound of formula (I) or a pharmaceutically acceptable salt or solvate thereof is useful in treating gilbert's syndrome, In embodiments, the therapeutic effective amount of Compound of formula (I) or a pharmaceutically acceptable salt or solvate thereof is useful in treating intrahepatic cholestasis of pregnancy. In embodiments, the therapeutic effective amount of Compound of formula (I) or a pharmaceutically acceptable salt or solvate thereof is useful in treating liver rejection. In embodiments, the therapeutic effective amount of Compound of formula (I) or a pharmaceutically acceptable salt or solvate thereof is useful in treating non-alcoholic fatty liver disease. In embodiments, the therapeutic effective amount of Compound of formula (I) or a pharmaceutically acceptable salt or solvate thereof is useful in treating non-alcoholic steatohepatitis. In embodiments, the therapeutic effective amount of Compound of formula (I) or a pharmaceutically acceptable salt or solvate thereof is useful in treating polycystic liver disease. In embodiments, the therapeutic effective amount of Compound of formula (I) or a pharmaceutically acceptable salt or solvate thereof is useful in treating PBC. In embodiments, the therapeutic effective amount of Compound of formula (I) or a pharmaceutically acceptable salt or solvate thereof is useful in treating PSC. In embodiments, the therapeutic effective amount of Compound of formula (I) or a pharmaceutically acceptable salt or solvate thereof is useful in treating veno-occlusive disease. In embodiments, the therapeutic effective amount of Compound of formula (I) or a pharmaceutically acceptable salt or solvate thereof is useful in treating wilson's disease, Ulcerative colitis, Crohn's disease, Irritable bowel syndrome (IBS), Proctitis, Celiac disease, ileitis, duodenitis, and diverticulitis.

In embodiments, the therapeutic effective amount of Compound 1 or a pharmaceutically acceptable salt or solvate thereof is useful in treating acute hepatitis. In embodiments, the therapeutic effective amount of Compound 1 or a pharmaceutically acceptable salt or solvate thereof is useful in treating alcoholic hepatitis. In embodiments, the therapeutic effective amount of Compound 1 or a pharmaceutically acceptable salt or solvate thereof is useful in treating alcoholic liver disease. In embodiments, the therapeutic effective amount of Compound 1 or a pharmaceutically acceptable salt or solvate thereof is useful in treating extrahepatic cholestasis. In embodiments, the therapeutic effective amount of Compound 1 or a pharmaceutically acceptable salt or solvate thereof is useful in treating hepatic fibrosis. In embodiments, the therapeutic effective amount of Compound 1 or a pharmaceutically acceptable salt or solvate thereof is useful in treating liver abscess. In embodiments, the therapeutic effective amount of Compound 1 or a pharmaceutically acceptable salt or solvate thereof is useful in treating liver cirrhosis. In embodiments, the therapeutic effective amount of Compound 1 or a pharmaceutically acceptable salt or solvate thereof is useful in treating neonatal jaundice. In embodiments, the therapeutic effective amount of Compound 1 or a pharmaceutically acceptable salt or solvate thereof is useful in treating obstructive jaundice. In embodiments, the therapeutic effective amount of Compound 1 or a pharmaceutically acceptable salt or solvate thereof is useful in treating primary bile acid malabsorption. In embodiments, the therapeutic effective amount of Compound 1 or a pharmaceutically acceptable salt or solvate thereof is useful in treating acute cholecystitis. In embodiments, the therapeutic effective amount of Compound 1 or a pharmaceutically acceptable salt or solvate thereof is useful in treating autoimmune hepatitis. In embodiments, the therapeutic effective amount of Compound 1 or a pharmaceutically acceptable salt or solvate thereof is useful in treating benign recurrent intrahepatic cholestasis. In embodiments,

the therapeutic effective amount of Compound 1 or a pharmaceutically acceptable salt or solvate thereof is useful in treating biliary obstruction. In embodiments, the therapeutic effective amount of Compound 1 or a pharmaceutically acceptable salt or solvate thereof is useful in treating cholestatic liver disease. In embodiments, the therapeutic effective amount of Compound 1 or a pharmaceutically acceptable salt or solvate thereof is useful in treating crigler-najjar syndrome. In embodiments, the therapeutic effective amount of Compound 1 or a pharmaceutically acceptable salt or solvate thereof is useful in treating drug-induced hepatitis. In embodiments, the therapeutic effective amount of Compound 1 or a pharmaceutically acceptable salt or solvate thereof is useful in treating gilbert's syndrome, In embodiments, the therapeutic effective amount of Compound 1 or a pharmaceutically acceptable salt or solvate thereof is useful in treating intrahepatic cholestasis of pregnancy, In embodiments, the therapeutic effective amount of Compound 1 or a pharmaceutically acceptable salt or solvate thereof is useful in treating liver rejection. In embodiments, the therapeutic effective amount of Compound 1 or a pharmaceutically acceptable salt or solvate thereof is useful in treating non-alcoholic fatty liver disease. In embodiments, the therapeutic effective amount of Compound 1 or a pharmaceutically acceptable salt or solvate thereof is useful in treating nonalcoholic steatohepatitis. In embodiments, the therapeutic effective amount of Compound 1 or a pharmaceutically acceptable salt or solvate thereof is useful in treating polycystic liver disease. In embodiments, the therapeutic effective amount of Compound 1 or a pharmaceutically acceptable salt or solvate thereof is useful in treating PBC. In embodiments, the therapeutic effective amount of Compound 1 or a pharmaceutically acceptable salt or solvate thereof is useful in treating PSC. In embodiments, the therapeutic effective amount of Compound 1 or a pharmaceutically acceptable salt or solvate thereof is useful in treating colitis. In embodiments, the therapeutic effective amount of Compound 1 or a pharmaceutically acceptable salt or solvate thereof is useful in treating Ulcerative colitis (UC) and/or Crohn's disease (CD). In embodiments, the therapeutic effective amount of Compound 1 or a pharmaceutically acceptable salt or solvate thereof is useful in treating veno-occlusive disease. In embodiments, the therapeutic effective amount of Compound 1 or a pharmaceutically acceptable salt or solvate thereof is useful in treating wilson's disease. In some embodiments, the disclosure encompasses a method for treating or preventing IBD in subjects in need thereof, said method comprising administering to said subject a therapeutic effective amount of the compounds of the present invention, including compound 1 or a pharmaceutically acceptable salt or solvate thereof. In embodiments, the disclosure encompasses a method for treating or preventing PSC in subjects suffering with IBD in need thereof, said method comprising administering to said subject a therapeutic effective amount of a compound 1 or a pharmaceutically acceptable salt or solvate thereof.

In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating acute hepatitis. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating alcoholic hepatitis. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating alcoholic liver disease. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating extrahepatic cholestasis. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating hepatic fibrosis. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating liver abscess. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating liver cirrhosis. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating neonatal jaundice. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating obstructive jaundice. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating primary bile acid malabsorption. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating acute cholecystitis. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating autoimmune hepatitis. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating benign recurrent intrahepatic cholestasis, In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating biliary obstruction. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating cholestatic liver disease. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating crigler-najjar syndrome. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating drug-induced hepatitis. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating gilbert's syndrome. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating intrahepatic cholestasis of pregnancy. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating liver rejection. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating non-alcoholic fatty liver disease. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating non-alcoholic steatohepatitis. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating polycystic liver disease. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating primary biliary cirrhosis. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating veno-occlusive disease. In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating wilson's disease.

### Other Diseases:

In embodiments, the method of the present disclosure is useful for the treatment of various EGID. In embodiments, EGID includes eosinophilic gastroenteritis and eosinophilic esophagitis, eosinophilic duodenitis, eosinophilic gastritis.

In embodiments, the therapeutic effective amount of Compound of formula (I) or a pharmaceutically acceptable salt or solvate thereof is useful in treating eosinophilic gastroenteritis. In embodiments, the therapeutic effective amount of Compound of formula (I) or a pharmaceutically acceptable salt or solvate thereof is useful in treating eosinophilic esophagitis. In embodiments, the therapeutic effective amount of Compound of formula (I) or a pharmaceutically acceptable salt or solvate thereof is useful in treating eosinophilic duodenitis. In embodiments, the therapeutic effective amount of Compound of formula (I) or a pharmaceutically acceptable salt or solvate thereof is useful in treating eosinophilic gastritis.

In embodiments, a therapeutic effective amount of Compound 1 or a pharmaceutically acceptable salt or a solvate thereof is useful in treating eosinophilic gastroenteritis. In embodiments, the therapeutic effective amount of Compound I or a pharmaceutically acceptable salt or a solvate thereof is useful in treating eosinophilic esophagitis. In embodiments, the therapeutic effective amount of Compound I or a pharmaceutically acceptable salt or solvate thereof is useful in treating eosinophilic duodenitis. In embodiments, a therapeutic effective amount of Compound 1 or a pharmaceutically acceptable salt or a solvate thereof is useful in treating eosinophilic gastritis.

In embodiments, a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating eosinophilic gastroenteritis. In embodiments, the therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating eosinophilic esophagitis. In embodiments, the therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating eosinophilic duodenitis. In embodiments, the therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate is useful in treating eosinophilic gastritis.

### SPECIFIC EMBODIMENTS OF THE PRESENT DISCLOSURE

Embodiment 1: A method for treating or preventing hepatobiliary disease in a subject in need thereof, said method comprising administering to said subject a therapeutic effective amount of a compound according to Formula (I) or a pharmaceutically acceptable salt or solvate thereof:

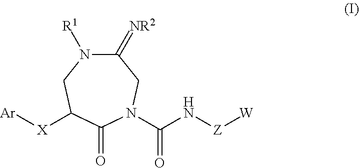

wherein,
Ar is (1) a C6 to C14 aromatic hydrocarbon group, (2) a 5 to 8-membered aromatic heterocyclic group having 1 to 4 hetero atoms selected from the group consisting of a nitrogen atom, sulfur atom, and oxygen atom, in place of a carbon atom, or (3) a bicyclic or tricyclic aromatic group formed by condensation of the above aromatic heterocyclic group and a C6 to C14 aromatic hydrocarbon ring,
where the above groups (1) to (3) of Ar are optionally substituted with 1 to 5 groups selected from the group consisting of:
(i) a halogen atom,
(ii) nitro,
(iii) cyano,
(iv) C1 to C6 alkyl optionally substituted with 1 to 3 halogen atoms,
(v) C2 to C6 alkenyl optionally substituted with 1 to 3 halogen atoms,
(vi) C2 to C6 alkynyl optionally substituted with 1 to 3 halogen atoms,
(vii) C3 to C6 cycloalkyl,
(viii) hydroxyl,
(ix) C1 to C6 alkoxy optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(x) C1 to C5 alkylenedioxy,
(xi) C1 to C6 alkylthio optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(xii) amino,
(xiii) mono-C1 to C6 alkylamino,
(xiv) di-C1 to C6 alkylamino,
(xv) 5- to 6-membered cyclic amino,
(xvi) C1 to C6 alkylcarbonyl,
(xvii) carboxyl,
(xviii) C1 to C6 alkoxycarbonyl,
(xix) carbamoyl,
(xx) thiocarbamoyl,
(xxi) mono-C1 to C6 alkylcarbamoyl,
(xxii) di-C1 to C6 alkylcarbamoyl,
(xxiii) 5- to 6-membered cyclic aminocarbonyl,
(xxiv) sulfo,
(xxv) C1 to C6 alkylsulfonyl
(xxvi) C1 to C6 alkoxycarbonylamino,
(xxvii) C1 to C6 alkylcarbonylamino,
(xxviii) mono- or di-C1 to C6 alkylaminocarbonylamino,
(xxix) aminosulfonyl, and
(xxx) mono- or di-C1 to C6 alkylaminosulfonyl, or substituted with 1 to 9 deuterium atoms,
X is (1) a connecting bond, (2) linear or branched C1 to C6 alkylene optionally substituted with 1 to 12 deuterium atoms, (3) an oxygen atom, (4) NR³, where R³ is a hydrogen atom or a C1 to C6 alkyl group, or (5) —S(O)m-, where m is an integer of 0 to 2,
Z is (1) a connecting bond or (2) CR⁴R⁵ where R⁴ and R⁵ are, independently,
(A) a hydrogen atom,
(B) a deuterium atom,
(C) C1 to C6 alkyl optionally substituted with 1 to 5 groups selected from the group consisting of (i) carboxyl, (ii) C1 to C6 alkoxycarbonyl, (iii) phenyl, (iv) hydroxyl, (v) C1 to C6 alkoxy, (vi) halogen atom, and (vii) C3 to C6 cycloalkyl, or substituted with 1 to 13 deuterium atoms,
(D) C3 to C6 cycloalkyl optionally substituted with 1 to 5 groups selected from the group consisting of (i) a halogen atom and (ii) an alkyl group optionally substituted with 1 to 3 halogen atoms or optionally substituted, with 1 to 11 deuterium atoms,
(E) COOR⁶, where R⁶ is a hydrogen atom or C1 to C6 alkyl, or
(F) CONR⁷R⁸, where R⁷ and R⁸ are, independently,
(a) hydrogen atom,
(b) C1 to C6 alkyl optionally substituted with 1 to 3 groups selected from the group consisting of (i) a halogen atom, (ii) C3 to C6 cycloalkyl, (iii) carboxyl, (iv) C1 to C6 alkoxycarbonyl, (v) C1 to C6 alkylcarbonyl, (vi) carbamoyl, (vii) mono-C1 to C6 alkylcarbamoyl, (viii) di-C1 to C6 alkylcarbamoyl, (ix) C6 to C12 aryl, and (x) C1 to C10 heteroaryl,
(c) C6 to C14 aromatic hydrocarbon group,
(d) a 5- to 8-membered aromatic heterocyclic group having 1 to 4 hetero atoms selected from the group consisting of a nitrogen atom, sulfur atom, and oxygen atom, in place of a carbon atom, or
(e) a bicyclic or tricyclic aromatic group formed by condensation of the above aromatic heterocyclic, group and a C6 to C14 aromatic hydrocarbon ring,
where each of the groups (c) to (e) is optionally substituted with 1 to 5 groups selected from the group consisting of (i) a halogen atom, (ii) nitro, (iii) cyano, (iv) C1 to C6 alkyl optionally substituted with 1 to 3 halogen atoms, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 halogen atoms, (x) C1 to C5 alkylenedioxy, (xi) C1 to C6 alkylthio optionally substituted with 1 to 3 halogen atoms, (xii) amino, (xiii) mono-C1 to C6 alkylamino, (xiv) di-C1 to C6 alkylamino, (xv) 5- to 6-membered cyclic amino, (xvi) C1 to C6 alkylcarbonyl, (xvii) carboxyl, (xviii) C1 to C6 alkoxycarbonyl, (xix) carbamoyl, (xx) thiocarbamoyl, (xxi) mono-C1 to C6 alkylcarbamoyl, (xxii) di-C1 to C6 alkylcarbamoyl, (xxiii) C6 to C10 arylcarbamoyl, (xxiv) C1 to C10 heteroarylcarbamoyl, (xxv) sulfo, (xxvi) C1 to C6 alkylsulfonyl, (xxvii) aminosulfonyl, and (xxviii) mono- or di-C1 to C6 alkylaminosulfonylamino, or substituted with 1 to 9 deuterium atoms,
W is (1) a hydrogen atom, (2) a C6 to C14 aromatic hydrocarbon group, (3) a 5- to 8-membered aromatic heterocyclic group having 1 to 4 hetero atoms selected from a nitrogen atom, a sulfur atom, and an oxygen atom, in place of a carbon atom, (4) a bicyclic, or tricyclic aromatic group formed by condensation of the above aromatic heterocyclic group and a C6 to C14 aromatic hydrocarbon ring, or a deuterium atom,
where, each of the groups (2) to (4) of the above W is optionally substituted with 1 to 5 groups selected from the group consisting of (i) a halogen atom, (ii) nitro, (iii) cyano, (iv) C1 to C6 alkyl optionally substituted with 1 to 3 groups selected from the group consisting of a halogen atom, amino, C1 to C6 alkoxycarbonyl, C1 to C6 alkoxycarbonylamino, and carboxyl, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 groups selected from the group consisting of a halogen atom, hydroxyl, C1 to C6 alkoxy, amino and mono- or di-C1 to C6 alkylamino, (x) C1 to C5 alkylenedioxy, (xi) C1 to C6 alkylthio optionally substituted with 1 to 3 groups selected from the group consisting of a halogen atom, hydroxyl, C1 to C6 alkoxy, amino, and mono- or di-C1 to C6 alkylamino, (xii) amino, (xiii) mono-C1 to C6 alkylamino, (xiv) di-C1 to C6 alkylamino, (xv) 5- to 6-membered cyclic amino, (xvi) C1 to C6 alkyl-carbonyl, (xvii) carboxyl, (xviii) C1 to C6 alkoxycarbonyl optionally substituted with a halogen atom, (xix) C7 to C16 aralkyloxycarbonyl optionally substituted with a halogen atom, (xx) carbamoyl, (xxi) mono-C1 to C6 alkylcarbamoyl optionally substituted with 1 to 3 groups which are selected from the group consisting of a halogen atom, hydroxyl, carboxyl, C1 to C6 alkoxy, amino, and mono- or di-C1 to C6 alkylamino, (xxii) di-C1 to C6 alkylcarbamoyl optionally substituted with hydroxyl,
(xxiii) 5- to 6-membered cyclic aminocarbonyl optionally substituted with C1 to C6 alkoxycarbonyl, (xxiv) C6 to C10 arylcarbamoyl, (xxv) C1 to C10 heteroarylcarbamoyl, (xxvi) C7 to C16 aralkylcarbamoyl, (xxvii) C1 to C10 heteroaryl-C1 to C6 alkylcarbamoyl, (xxviii) N C1 to C6 alkyl-N—C6 to C12 arylcarbamoyl, (xxix) C3 to C6 cycloalkylcarbamoyl, (xxx) sulfo, (xxxi) C1 to C6 alkylsulfonyl, (xxxii) C1 to C6 alkylsulfonylamino, (xxxiii) C6 to C12 arylsulfonylamino optionally substituted with C1 to C6 alkyl, (xxxiv) C1 to C10 heteroarylsulfonylamino, (xxxv) C1 to C6 alkoxycarbonylamino, (xxxvi) C1 to C6 alkylcarbonylamino, (xxxvii) mono- or di-C1 to C6 alkylaminocarbonylamino, (xxxviii) C6 to C12 aryl, (xxxix) C1 to C10 heteroaryl, (xl) C6 to C10 aryloxy, (xli) C1 to C10 heteroaryloxy, (xlii) C7 to C16 aralkyloxy, (xliii) C1 to C10 heteroaryl-C1 to C6 alkyloxy, (xliv) aminosulfonyl, (xlv) mono- or di-C1 to C6 alkylaminosulfonyl, (xlvi) C7 to C16 aralkyloxycarbamoyl, and (xlvii) C1 to C10 heteroaryl-C1 to C6 alkyloxycarbamoyl, or substituted with 1 to 9 deuterium atoms,
R1 is
(1) a hydrogen atom,
(2) C1 to C6 alkyl optionally substituted with 1 to 3 of (i) a halogen atom or (ii) C3 to C6 cycloalkyl or substituted with (iii) 1 to 13 deuterium atoms,
(3) C2 to C6 alkenyl optionally substituted with 1 to 3 of (i) a halogen atom or (ii) C3 to C6 cycloalkyl or substituted with (iii) 1 to 11 deuterium atoms,
(4) C2 to C6 alkynyl optionally substituted with 1 to 3 of (i) a halogen atom or (ii) C3 to C6 cycloalkyl, or substituted with (iii) 1 to 9 deuterium atoms, or
(5) C3 to C6 cycloalkyl optionally substituted with 1 to 3 of (i) a halogen atom or (ii) C3 to C6 cycloalkyl, or substituted with (iii) 1 to 11 deuterium atoms,
R2 is (1) OR⁹ or (2) NR¹⁰R¹¹
where, R⁹, R¹⁰, and R¹¹ independently indicate
(A) a hydrogen atom,
(B) C1 to C6 alkyl,
(C) C2 to C6 alkenyl,
(D) C3 to C6 alkynyl,
(E) C3 to C6 cycloalkyl
(F) C6 to C14 aromatic hydrocarbon group,
(G) a 5- to 8-membered aromatic heterocyclic group having 1 to 4 hetero atoms selected from the group consisting of a nitrogen atom, sulfur atom, and oxygen atom, in place of a carbon atom, or
(H) a bicyclic or tricyclic aromatic group formed by condensation of the above aromatic heterocyclic group and a C6 to C14 aromatic hydrocarbon ring,
where each of the groups of the above (B) to (E) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5 to 6-membered cyclic amino, (ix) carboxyl, (x) C1 to C6 alkoxycarbonyl, (xi) C1 to C6 alkylcarbonyl, (xii) carbamoyl, (xiii) mono-C1 to C6 alkylcarbamoyl, (xiv) di-C1 to C6 alkylcarbamoyl, (xv) C6 to C12 aryl, and (xvi) C1 to C10 heteroaryl, or substituted with 1 to 13 deuterium atoms, further, each of the groups of the above (F) to (H) are optionally substituted with 1 to 5 groups which are selected from the group consisting of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms, or R¹ and R² may form 5- or 6-member heterocyclic ring together with the atoms they are bonded with, where the above 5- or 6-member heterocyclic ring is optionally substituted with 1 to 3 groups selected from the group consisting of
(A) a halogen atom,
(B) oxo,
(C) hydroxyl
(D) C1 to C6 alkyl,
(E) C2 to C6 alkenyl,
(F) C2 to C6 alkynyl,
(G) C3 to C6 cycloalkyl
(H) C6 to C14 aromatic hydrocarbon group,
(I) 5- to 8-membered aromatic heterocyclic group having 1 to 4 hetero atoms selected from the group consisting of a nitrogen atom, sulfur atom, and oxygen atom, in place of a carbon atom, and
(J) a bicyclic or tricyclic aromatic group formed by condensation of the above aromatic heterocyclic group and a C6 to C14 aromatic hydrocarbon ring, or substituted with
(K) 1 to 6 deuterium atoms,
where each of the groups of the above (D) to (G) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5- to 6-membered cyclic amino, (ix) carboxyl, (x) C1 to C6 alkoxycarbonyl, (xi) C1 to C6 alkyl-carbonyl, (xii) carbamoyl, (xiii) mono-C1 to C6 alkylcarbamoyl, (xiv) di-C1 to C6 alkylcarbamoyl, (xv) C6 to C12 aryl and (xvi) C1 to C10 heteroaryl, or substituted with 1 to 13 deuterium atoms, each of the groups of the above (H) to (J) is optionally substituted with 1 to 5 groups which are selected from the group consisting, of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms.

Embodiment 2: The method according to embodiment 1, wherein the compound of Formula (I) is selected from the group consisting of:
(1) 4-{(1R)-1-[({(6R)-6-(5-chloro-2-methoxybenzyl)-7-oxo-3-[(pyridin-2-yloxy)imino]-1,4-diazepan-1-yl}carbonyl)amino]propyl}benzoic acid,
(2) 2-amino-4-[(1R)-1-({[(6S)-6-(5-chloro-2-methoxybenzyl)-3-(ethoxyimino)-7-oxo-1,4-diazepan-1-yl]carbonyl}amino)butyl]benzoic acid,
(3) 2-amino-4-{(1R)-1-[({(6S)-6-(5-chloro-2-methoxybenzyl)-7-oxo-3-[(2,2,2-trifluoroethoxy)imino]-1,4-diazepan-1-yl}carbonyl)amino]butyl}benzoic acid,
(4) 2-amino-4-{(1R)-1-[({(6R)-6-(5-chloro-2-methoxybenzyl)-3-[(3,5-difluorophenoxy)imino]-7-oxo-1,4-diazepan-1-yl}carbonyl)amino]ethyl}benzoic acid, (5) 2-amino-4-[(1R)-1-({[(6S)-6-(5-chloro-2-methoxybenzyl)-3-(methoxyimino)-4-methyl-7-oxo-1,4-diazepan-1-yl]carbonyl}amino)butyl]benzoic acid,
(6) (4-{(1R)-1-[({(6R)-6-(5-chloro-2-methoxybenzyl)-3-[(3,5-difluorophenoxy)imino]-7-oxo-1,4-diazepan-1-yl}carbonyl)amino]ethyl}phenyl) acetic acid,
(7) 2-amino-4-{[({(6R)-6-(5-chloro-2-methoxybenzyl)-3-[(3,5-difluorophenoxy)imino]-7-oxo-1,4-diazepan-1-yl}carbonyl)amino]methyl}benzoic acid,
(8) {4-[(1R)-1-({[(6R)-6-(5-chloro-2-methoxybenzyl)-7-oxo-3-(phenoxyimino)-1,4-diazepan-1-yl]carbonyl}amino)propyl]phenyl}acetic acid,
(9) 2-amino-4-[(1R)-1-({[(6R)-6-(5-chloro-2-methoxybenzyl)-7-oxo-3-(phenoxyimino)-1,4-diazepan-1-yl]carbonyl}amino)propyl]benzoic acid, or
(10) 2-amino-4-[(1R)-1({[(6R)-6-(5-chloro-2-methoxybenzyl)-3-(ethoxyimino)-7-oxo-1,4-diazepan-1-yl]carbonyl}amino)butyl]benzoic acid.

Embodiment 3: The method according to embodiment 1 or 2, wherein the compound of Formula (I) is 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid (compound 1) or a pharmaceutically acceptable salt or solvate thereof,

Embodiment 4: The method according to embodiment 3, wherein compound 1 is monoacetic acid solvate.

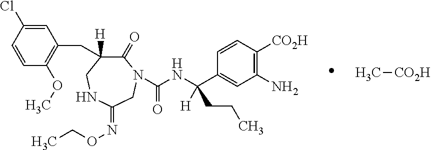

Embodiment 5: The method according to embodiment 1, wherein the compound of Formula I or a pharmaceutically acceptable salt or solvate thereof is selective for chymase over cathepsin G.

Embodiment 6: The method according to embodiment 1, wherein the compound of Formula I or a pharmaceutically acceptable salt or solvate thereof has lesser side effects to non-selective chymase inhibitors after prolonged use in treating hepatobiliary disease.

Embodiment 7: The method according to embodiment 1, wherein the administration of therapeutic effective amount of compound of formula (I) or a pharmaceutically acceptable salt or a solvate thereof ameliorates jaundice.

Embodiment 8: The method according to embodiment 1, wherein the administration of therapeutic effective amount of compound of formula (I) or a pharmaceutically acceptable salt or solvate thereof ameliorates pruritis.

Embodiment 9: The method according to embodiment 1, wherein the administration of therapeutic effective amount of compound of formula (I) or a pharmaceutically acceptable salt or solvate thereof ameliorates inflammation.

Embodiment 10: The method according to any of the embodiments 1-9, wherein the hepatobiliary disease is selected from the group consisting of acute hepatitis, alcoholic hepatitis, alcoholic liver disease, extrahepatic cholestasis, hepatic fibrosis, liver abscess, liver cirrhosis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, non-alcoholic steatohepatitis, polycystic liver disease, PBC, PSC, veno-occlusive disease, and Wilson's disease.

Embodiment 11: A method for treating or preventing hepatobiliary disease in a subject in need thereof, said method comprising administering to said subject a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid (compound 1) or a pharmaceutically acceptable salt or solvate thereof.

Embodiment 12: A method for reducing mast cell infiltration in a subject suffering from hepatic biliary disease, said method comprising administering to said subject a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid (compound 1) or a pharmaceutically acceptable salt or solvate thereof.

Embodiment 13: A method for treating or preventing pruritis in a subject suffering from hepatic biliary disease, said method comprising administering to said subject a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid (compound 1) or a pharmaceutically acceptable salt or solvate thereof.

Embodiment 14: A method for treating or preventing jaundice in a subject suffering from hepatic biliary disease, said method comprising administering to said subject a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid (compound 1) or a pharmaceutically acceptable salt or solvate thereof.

Embodiment 15: A method for reducing inflammation in a subject suffering from hepatic biliary disease, said method comprising administering to said subject a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid (compound 1) or a pharmaceutically acceptable salt or solvate thereof.

Embodiment 16: A method for reducing bile acid accumulation in a subject suffering from hepatic biliary disease, said method comprising administering to said subject a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid (compound 1) or a pharmaceutically acceptable salt or solvate thereof.

Embodiment 17: The method according to any of the embodiments 1-16, wherein the hepatobiliary disease is selected from the group consisting of acute hepatitis, alcoholic hepatitis, alcoholic liver disease, extrahepatic cholestasis, hepatic fibrosis, liver abscess, liver cirrhosis, neonatal jaundice, obstructive jaundice, colitis, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, non-alcoholic steatohepatitis, polycystic liver disease, PBC, PSC, veno-occlusive disease, Wilson's disease.

Embodiment 18: The method according to embodiment 17, wherein the hepatobiliary disease is PSC or PBC.

Embodiment 19: The method according to embodiment 17, wherein the hepatobiliary disease is associated with various gastrointestinal disorders, including colitis, Ulcerative colitis, Crohn's disease, Irritable bowel syndrome (IBS), Proctitis, Celiac disease, ileitis, duodenitis, and diverticulitis.

Embodiment 20: The method according to any of embodiments 8-18, wherein the therapeutic effective amount of Compound 1 is from about 10 mg to about 1000 mg per unit dose (e.g., about 50 mg to about 950 mg per unit dose, about 10 mg to about 900 mg per unit dose, about 10 mg to about 850 mg per unit dose, about 10 mg to about 800 mg per unit dose, about 10 mg to about 750 mg per unit dose, about 10 mg to about 700 mg per unit dose, about 10 mg to about 650 mg per unit dose, about 10 mg to about 600 mg per unit dose, about 50 mg to about 550 mg per unit dose, about 50 mg to about 500 mg per unit dose, about 50 mg to about 450 mg per unit dose, about 50 mg to about 400 mg per unit dose, about 50 mg to about 350 mg per unit dose, about 50 mg to about 300 mg per unit dose, about 50 mg to about 250 mg per unit dose, about 50 mg to about 200 mg per unit dose, about 50 mg to about 150 mg per unit dose, about 50 mg to about 100 mg per unit dose).

Embodiment 21: The method according to any of embodiments 11-16, wherein the therapeutic effective amount of compound 1 or a pharmaceutically acceptable salt thereof are administered once a day, twice a day, thrice a day, once in two days, or once in three days or once a week.

Embodiment 22: The method according to any of embodiments 11-16, wherein the therapeutic effective amount of compound 1 or a pharmaceutically acceptable salt or solvate thereof are administered orally to the subject.

Embodiment 23. The method according to embodiment 22, wherein the therapeutic effective amount of compound 1 or a pharmaceutically acceptable salt or solvate thereof are administered in unit dosage form for oral route formulated as a pellet, multiunit particulate system (MUPS), tablets, troches, lozenges, dispersible powders or granules, or a hard or soft capsule, powders, sustained-release formulations, microcapsules.

Embodiment 24: The method according to any of the previous embodiment, wherein the subject is mammal preferably human.

Embodiment 25: A method for treating PSC in a subject, said method comprising administering to said subject 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid monoacetic acid in a range of about 10 mg to about 1000 mg per unit dose.

Embodiment 26: A method for reducing biliary senescence in a subject suffering from PSC, said method comprising administering to said subject a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid monoacetic acid.

Embodiment 27: A method for reducing proliferation of ductular hepatocytes in a subject suffering from PSC, said method comprising administering to said subject a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid monoacetic acid.

Embodiment 28: A method for reducing angiogenesis in a subject suffering from PSC, said method comprising administering to said subject a therapeutic effective amount of 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid monoacetic acid.

Embodiment 29: A method for treating PBC in a subject, said method comprising administering to said subject 2-amino-4-[(1R)-1-({[(3Z,6S)-6-(5-chloro-2-methoxybenzyl)-3-(ethoxyimino)-7-oxo-1,4-diazepan-1-yl]carbonyl}amino) butyl]benzoic acid monoacetic acid in a range of about 10 mg to about 1000 mg per unit.

Embodiment 30: A pharmaceutical composition for treating hepatobiliary disease in a subject comprising a therapeutic effective amount of compound 1 or a pharmaceutically acceptable salt or solvate thereof.

Embodiment 31: The pharmaceutical composition of embodiment 30, wherein said therapeutic effective amount of compound 1 or a pharmaceutically acceptable salt or solvate thereof is from about 10 mg to about 1000 mg per unit dose.

Embodiment 32: The pharmaceutical composition of embodiment 30, wherein hepatobiliary disease is selected from the group comprising of acute hepatitis, alcoholic hepatitis, alcoholic liver disease, extrahepatic cholestasis, hepatic fibrosis, liver abscess, liver cirrhosis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, non-alcoholic steatohepatitis, polycystic liver disease, PBC, PSC, veno-occlusive disease, Wilson's disease.

Embodiment 33: The pharmaceutical composition of embodiment 30, wherein hepatobiliary disease is PSC or PBC.

Embodiment 34: The pharmaceutical composition of embodiment 30, wherein hepatobiliary disease is colitis.

Embodiment 35: A pharmaceutical composition for treating PSC or PBC in a subject comprising about 10 mg to about 1000 mg of compound 1 or a pharmaceutically acceptable salt thereof and optionally pharmaceutically acceptable carrier or excipient.

Embodiment 36: A pharmaceutical composition for treating colitis in a subject comprising about 10 mg to about 1000 mg of compound 1 or a pharmaceutically acceptable salt thereof and optionally pharmaceutically acceptable carrier or excipient

Embodiment 37: A method for treating or preventing EGID in a subject in need thereof, said method comprising administering to said subject a therapeutic effective amount of compound of Formula (I) or a pharmaceutically acceptable salt or solvate thereof:

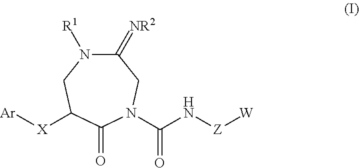

wherein,
Ar is (1) a C6 to C14 aromatic hydrocarbon group, (2) a 5 to 8-membered aromatic heterocyclic group having 1 to 4 hetero atoms selected from the group consisting of a nitrogen atom, sulfur atom, and oxygen atom, in place of a carbon atom, or (3) a bicyclic or tricyclic aromatic group formed by condensation of the above aromatic heterocyclic group and a C6 to C14 aromatic hydrocarbon ring,
where the above groups (1) to (3) of Ar are optionally substituted with 1 to 5 groups selected from the group consisting of:
(i) a halogen atom,
(ii) nitro,
(iii) cyano,
(iv) C1 to C6 alkyl optionally substituted with 1 to 3 halogen atoms,
(v) C2 to C6 alkenyl optionally substituted with 1 to 3 halogen atoms,
(vi) C2 to C6 alkynyl optionally substituted with 1 to 3 halogen atoms,
(vii) C3 to C6 cycloalkyl,
(viii) hydroxyl,
(ix) C1 to C6 alkoxy optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(x) C1 to C5 alkylenedioxy,
(xi) C1 to C6 alkylthio optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(xii) amino,
(xiii) mono-C1 to C6 alkylamino,
(xiv) di-C1 to C6 alkylamino,
(xv) 5- to 6-membered cyclic amino,
(xvi) C1 to C6 alkylcarbonyl,
(xvii) carboxyl,
(xviii) C1 to C6 alkoxycarbonyl,
(xix) carbamoyl,
(xx) thiocarbamoyl,
(xxi) mono-C1 to C6 alkylcarbamoyl,
(xxii) di-C1 to C6 alkylcarbamoyl,
(xxiii) 5- to 6-membered cyclic aminocarbonyl,
(xxiv) sulfo,
(xxv) C1 to C6 alkylsulfonyl
(xxvi) C1 to C6 alkoxycarbonylamino,
(xxvii) C1 to C6 alkylcarbonylamino,
(xxviii) mono- or di-C1 to C6 alkylaminocarbonylamino,
(xxix) aminosulfonyl, and
(xxx) mono- or di-C1 to C6 alkylaminosulfonyl, or substituted with 1 to 9 deuterium atoms,
X is (1) a connecting bond, (2) linear or branched C1 to C6 alkylene optionally substituted with 1 to 12 deuterium atoms, (3) an oxygen atom, (4) NR³, where R³ is a hydrogen atom or a C1 to C6 alkyl group, or (5) —S(O)m-, where m is an integer of 0 to 2,
Z is (1) a connecting bond or (2) CR⁴R⁵ where R⁴ and R⁵ are, independently,
(A) a hydrogen atom,
(B) a deuterium atom,
(C) C1 to C6 alkyl optionally substituted with 1 to 5 groups selected from the group consisting of (i) carboxyl, (ii) C1 to C6 alkoxycarbonyl, (iii) phenyl, (iv) hydroxyl, (v) C1 to C6 alkoxy, (vi) halogen atom, and (vii) C3 to C6 cycloalkyl, or substituted with 1 to 13 deuterium atoms,
(D) C3 to C6 cycloalkyl optionally substituted with 1 to 5 groups selected from the group consisting of (i) a halogen atom and (ii) an alkyl group optionally substituted with 1 to 3 halogen atoms or optionally substituted, with 1 to 11 deuterium atoms,
(E) COOR⁶, where R⁶ is a hydrogen atom or C1 to C6 alkyl, or
(F) CONR⁷R⁸, where R⁷ and R⁸ are, independently,
(a) hydrogen atom,
(b) C1 to C6 alkyl optionally substituted with 1 to 3 groups selected from the group consisting of (i) a halogen atom, (ii) C3 to C6 cycloalkyl, (iii) carboxyl, (iv) C1 to C6 alkoxycarbonyl, (v) C1 to C6 alkylcarbonyl, (vi) carbamoyl, (vii) mono-C1 to C6 alkylcarbamoyl, (viii) di-C1 to C6 alkylcarbamoyl, (ix) C6 to C12 aryl, and (x) C1 to C10 heteroaryl,
(c) C6 to C14 aromatic hydrocarbon group,
(d) a 5- to 8-membered aromatic heterocyclic group having 1 to 4 hetero atoms selected from the group consisting of a nitrogen atom, sulfur atom, and oxygen atom, in place of a carbon atom, or
(e) a bicyclic or tricyclic aromatic group formed by condensation of the above aromatic heterocyclic, group and a C6 to C14 aromatic hydrocarbon ring,
where each of the groups (c) to (e) is optionally substituted with 1 to 5 groups selected from the group consisting of (i) a halogen atom, (ii) nitro, (iii) cyano, (iv) C1 to C6 alkyl optionally substituted with 1 to 3 halogen atoms, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 halogen atoms, (x) C1 to C5 alkylenedioxy, (xi) C1 to C6 alkylthio optionally substituted with 1 to 3 halogen atoms, (xii) amino, (xiii) mono-C1 to C6 alkylamino, (xiv) di-C1 to C6 alkylamino, (xv) 5- to 6-membered cyclic amino, (xvi) C1 to C6 alkylcarbonyl, (xvii) carboxyl, (xviii) C1 to C6 alkoxycarbonyl, (xix) carbamoyl, (xx) thiocarbamoyl, (xxi) mono-C1 to C6 alkylcarbamoyl, (xxii) di-C1 to C6 alkylcarbamoyl, (xxiii) C6 to C10 arylcarbamoyl, (xxiv) C1 to C10 heteroarylcarbamoyl, (xxv) sulfo, (xxvi) C1 to C6 alkylsulfonyl, (xxvii) aminosulfonyl, and (xxviii) mono- or di-C1 to C6 alkylaminosulfonylamino, or substituted with 1 to 9 deuterium atoms,
W is (1) a hydrogen atom, (2) a C6 to C14 aromatic hydrocarbon group, (3) a 5- to 8-membered aromatic heterocyclic group having 1 to 4 hetero atoms selected from a nitrogen atom, a sulfur atom, and an oxygen atom, in place of a carbon atom, (4) a bicyclic, or tricyclic aromatic group formed by condensation of the above aromatic heterocyclic group and a C6 to C14 aromatic hydrocarbon ring, or a deuterium atom,
where, each of the groups (2) to (4) of the above W is optionally substituted with 1 to 5 groups selected from the group consisting of (i) a halogen atom, (ii) nitro, (iii) cyano, (iv) C1 to C6 alkyl optionally substituted with 1 to 3 groups selected from the group consisting of a halogen atom, amino, C1 to C6 alkoxycarbonyl, C1 to C6 alkoxycarbonylamino, and carboxyl, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 groups selected from the group consisting of a halogen atom, hydroxyl, C1 to C6 alkoxy, amino and mono- or di-C1 to C6 alkylamino, (x) C1 to C5 alkylenedioxy, (xi) C1 to C6 alkylthio optionally substituted with 1 to 3 groups selected from the group consisting of a halogen atom, hydroxyl, C1 to C6 alkoxy, amino, and mono- or di-C1 to C6 alkylamino, (xii) amino, (xiii) mono-C1 to C6 alkylamino, (xiv) di-C1 to C6 alkylamino, (xv) 5- to 6-membered cyclic amino, (xvi) C1 to C6 alkyl-carbonyl, (xvii) carboxyl, (xviii) C1 to C6 alkoxycarbonyl optionally substituted with a halogen atom, (xix) C7 to C16 aralkyloxycarbonyl optionally substituted with a halogen atom, (xx) carbamoyl, (xxi) mono-C1 to C6 alkylcarbamoyl optionally substituted with 1 to 3 groups which are selected from the group consisting of a halogen atom, hydroxyl, carboxyl, C1 to C6 alkoxy, amino, and mono- or di-C1 to C6 alkylamino, (xxii) di-C1 to C6 alkylcarbamoyl optionally substituted with hydroxyl,
(xxiii) 5- to 6-membered cyclic aminocarbonyl optionally substituted with C1 to C6 alkoxycarbonyl, (xxiv) C6 to C10 arylcarbamoyl, (xxv) C1 to C10 heteroarylcarbamoyl, (xxvi) C7 to C16 aralkylcarbamoyl, (xxvii) C1 to C10 heteroaryl-C1 to C6 alkylcarbamoyl, (xxviii) N C1 to C6 alkyl-N—C6 to C12 arylcarbamoyl, (xxix) C3 to C6 cycloalkylcarbamoyl, (xxx) sulfo, (xxxi) C1 to C6 alkylsulfonyl, (xxxii) C1 to C6 alkylsulfonylamino, (xxxiii) C6 to C12 arylsulfonylamino optionally substituted with C1 to C6 alkyl, (xxxiv) C1 to C10 heteroarylsulfonylamino, (xxxv) C1 to C6 alkoxycarbonylamino, (xxxvi) C1 to C6 alkylcarbonylamino, (xxxvii) mono- or di-C1 to C6 alkylaminocarbonylamino, (xxxviii) C6 to C12 aryl, (xxxix) C1 to C10 heteroaryl, (xl) C6 to C10 aryloxy, (xli) C1 to C10 heteroaryloxy, (xlii) C7 to C16 aralkyloxy, (xliii) C1 to C10 heteroaryl-C1 to C6 alkyloxy, (xliv) aminosulfonyl, (xlv) mono- or di-C1 to C6 alkylaminosulfonyl, (xlvi) C7 to C16 aralkyloxycarbamoyl, and (xlvii) C1 to C10 heteroaryl-C1 to C6 alkyloxycarbamoyl, or substituted with 1 to 9 deuterium atoms,
R1 is
(1) a hydrogen atom,
(2) C1 to C6 alkyl optionally substituted with 1 to 3 of (i) a halogen atom or (ii) C3 to C6 cycloalkyl or substituted with (iii) 1 to 13 deuterium atoms,
(3) C2 to C6 alkenyl optionally substituted with 1 to 3 of (i) a halogen atom or (ii) C3 to C6 cycloalkyl or substituted with (iii) 1 to 11 deuterium atoms,
(4) C2 to C6 alkynyl optionally substituted with 1 to 3 of (i) a halogen atom or (ii) C3 to C6 cycloalkyl, or substituted with (iii) 1 to 9 deuterium atoms, or
(5) C3 to C6 cycloalkyl optionally substituted with 1 to 3 of (i) a halogen atom or (ii) C3 to C6 cycloalkyl, or substituted with (iii) 1 to 11 deuterium atoms,
R2 is (1) OR⁹ or (2) NR¹⁰R¹¹
where, R⁹, R¹⁰, and R¹¹ independently indicate
(A) a hydrogen atom,
(B) C1 to C6 alkyl,
(C) C2 to C6 alkenyl,
(D) C3 to C6 alkynyl,
(E) C3 to C6 cycloalkyl
(F) C6 to C14 aromatic hydrocarbon group,
(G) a 5- to 8-membered aromatic heterocyclic group having 1 to 4 hetero atoms selected from the group consisting of a nitrogen atom, sulfur atom, and oxygen atom, in place of a carbon atom, or
(H) a bicyclic or tricyclic aromatic group formed by condensation of the above aromatic heterocyclic group and a C6 to C14 aromatic hydrocarbon ring,
where each of the groups of the above (B) to (E) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5 to 6-membered cyclic amino, (ix) carboxyl, (x) C1 to C6 alkoxycarbonyl, (xi) C1 to C6 alkylcarbonyl, (xii) carbamoyl, (xiii) mono-C1 to C6 alkylcarbamoyl, (xiv) di-C1 to C6 alkylcarbamoyl, (xv) C6 to C12 aryl, and (xvi) C1 to C10 heteroaryl, or substituted with 1 to 13 deuterium atoms, further, each of the groups of the above (F) to (H) are optionally substituted with 1 to 5 groups which are selected from the group consisting of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms, or R¹ and R² may form 5- or 6-member heterocyclic ring together with the atoms they are bonded with, where the above 5- or 6-member heterocyclic ring is optionally substituted with 1 to 3 groups selected from the group consisting of
(A) a halogen atom,
(B) oxo,
(C) hydroxyl
(D) C1 to C6 alkyl,
(E) C2 to C6 alkenyl,
(F) C2 to C6 alkynyl,
(G) C3 to C6 cycloalkyl
(H) C6 to C14 aromatic hydrocarbon group,
(I) 5- to 8-membered aromatic heterocyclic group having 1 to 4 hetero atoms selected from the group consisting of a nitrogen atom, sulfur atom, and oxygen atom, in place of a carbon atom, and
(J) a bicyclic or tricyclic aromatic group formed by condensation of the above aromatic heterocyclic group and a C6 to C14 aromatic hydrocarbon ring, or substituted with
(K) 1 to 6 deuterium atoms,
where each of the groups of the above (D) to (G) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5- to 6-membered cyclic amino, (ix) carboxyl, (x) C1 to C6 alkoxycarbonyl, (xi) C1 to C6 alkyl-carbonyl, (xii) carbamoyl, (xiii) mono-C1 to C6 alkylcarbamoyl, (xiv) di-C1 to C6 alkylcarbamoyl, (xv) C6 to C12 aryl and (xvi) C1 to C10 heteroaryl, or substituted with 1 to 13 deuterium atoms, each of the groups of the above (H) to (J) is optionally substituted with 1 to 5 groups which are selected from the group consisting, of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms.

Embodiment 38: The method according to embodiment 37, wherein the compound of Formula (I) or a pharmaceutically acceptable salt or solvate thereof is selected from the group consisting of:
(1) 4-{(1R)-1-[({(6R)-6-(5-chloro-2-methoxybenzyl)-7-oxo-3-[(pyridin-2-yloxy)imino]-1,4-diazepan-1-yl}carbonyl)amino]propyl}benzoic acid,
(2) 2-amino-4-[(1R)-1-({[(6S)-6-(5-chloro-2-methoxybenzyl)-3-(ethoxyimino)-7-oxo-1,4-diazepan-1-yl]carbonyl}amino)butyl]benzoic acid,
(3) 2-amino-4-{(1R)-1-[({(6S)-6-(5-chloro-2-methoxybenzyl)-7-oxo-3-[(2,2,2-trifluoroethoxy)imino]-1,4-diazepan-1-yl}carbonyl)amino]butyl}benzoic acid,
(4) 2-amino-4-{(1R)-1-[({(6R)-6-(5-chloro-2-methoxybenzyl)-3-[(3,5-difluorophenoxy)imino]-7-oxo-1,4-diazepan-1-yl}carbonyl)amino]ethyl}benzoic acid,
(5) 2-amino-4-[(1R)-1-({[(6S)-6-(5-chloro-2-methoxybenzyl)-3-(methoxyimino)-4-methyl-7-oxo-1,4-diazepan-1-yl]carbonyl}amino)butyl]benzoic acid,
(6) (4-{(1R)-1-[({(6R)-6-(5-chloro-2-methoxybenzyl)-3-[(3,5-difluorophenoxy)imino]-7-oxo-1,4-diazepan-1-yl}carbonyl)amino]ethyl}phenyl) acetic acid,
(7) 2-amino-4-{[({(6R)-6-(5-chloro-2-methoxybenzyl)-3-[(3,5-difluorophenoxy)imino]-7-oxo-1,4-diazepan-1-yl}carbonyl)amino]methyl}benzoic acid,
(8) {4-[(1R)-1-({[(6R)-6-(5-chloro-2-methoxybenzyl)-7-oxo-3-(phenoxyimino)-1,4-diazepan-1-yl]carbonyl}amino)propyl]phenyl}acetic acid,
(9) 2-amino-4-[(1R)-1-({[(6R)-6-(5-chloro-2-methoxybenzyl)-7-oxo-3-(phenoxyimino)-1,4-diazepan-1-yl]carbonyl}amino)propyl]benzoic acid, or
(10) 2-amino-4-[(1R)-1({[(6R)-6-(5-chloro-2-methoxybenzyl)-3-(ethoxyimino)-7-oxo-1,4-diazepan-1-yl]carbonyl}amino)butyl]benzoic acid.

Embodiment 37: The method according to embodiment 30 or 31, wherein the compound of Formula (I) or a pharmaceutically acceptable salt or solvate thereof is 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid (compound 1)

Embodiment 38 The method according to embodiment 30 or 31, wherein the solvate of compound 1 is 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid

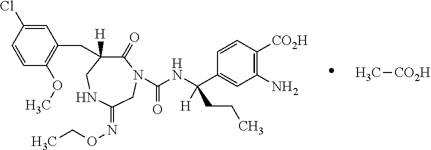

Embodiment 41: The method according to the embodiment 38, wherein the EGID is selected from the group comprising eosinophilic gastroenteritis and eosinophilic esophagitis, eosinophilic duodenitis, eosinophilic gastritis

Embodiment 42: A method for treating or preventing EGID in a subject in need thereof, said method comprising administering to said subject a therapeutic effective amount of compound 1 or a pharmaceutically acceptable salt or solvate thereof.

Embodiment 43: The method according to embodiment 42, wherein the EGID is selected from eosinophilic gastroenteritis and eosinophilic esophagitis, eosinophilic duodenitis, eosinophilic gastritis.

Embodiment 44: The method according to embodiment 42, wherein the therapeutic effective amount of Compound 1 is from about 10 mg to about 1000 mg per unit dose (e.g., about 50 mg to about 500 mg).

Embodiment 45. The method according to any of embodiments 42-44, wherein the therapeutic effective amount of compound 1 or a pharmaceutically acceptable salt thereof are administered once a day, twice a day, thrice a day, once in two days, or once in three days or once a week.

Embodiment 46. The method according to any of embodiments 42-44, wherein the therapeutic effective amount of compound 1 or a pharmaceutically acceptable salt thereof are administered orally to the subject.

Embodiment 47. The method according to embodiment 42-44, wherein the therapeutic effective amount of compound 1 or a pharmaceutically acceptable salt or solvate thereof are administered in unit dosage form for oral route formulated as a pellet, multiunit particulate system (MUPS), tablets, troches, lozenges, dispersible powders or granules, or a hard or soft capsule, powders, sustained-release formulations, microcapsules.

Embodiment 48: The method according to any of the previous embodiments, wherein the subject is mammal preferably human.

Embodiment 49: A pharmaceutical composition for treating EGID in a subject comprising a therapeutic effective amount of compound of formula (I) or a pharmaceutically acceptable salt or solvate thereof.

Embodiment 50: A pharmaceutical composition for treating EGID in a subject comprising a therapeutic effective amount of compound 1 or a pharmaceutically acceptable salt or solvate thereof.

Embodiment 51: The pharmaceutical composition of embodiment 48, wherein said therapeutic effective amount of compound 1 or a pharmaceutically acceptable salt or solvate thereof is from about 10 mg to about 1000 mg per unit dose.

Embodiment 52: The pharmaceutical composition of embodiment 49 or 50, wherein EGID is selected from the group consisting of eosinophilic gastroenteritis and eosinophilic esophagitis, eosinophilic duodenitis, eosinophilic gastritis.

Embodiment 53: A pharmaceutical composition for treating eosinophilic gastroenteritis in a subject comprising 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate and optionally pharmaceutically acceptable carrier or excipient.

Embodiment 54: A pharmaceutical composition for treating eosinophilic esophagitis in a subject comprising 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate and optionally pharmaceutically acceptable carrier or excipient.

Embodiment 55: A pharmaceutical composition for treating eosinophilic duodenitis in a subject comprising 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate and optionally pharmaceutically acceptable carrier or excipient.

Embodiment 56: A pharmaceutical composition for treating eosinophilic gastritis in a subject comprising 2-amino-4-[(1R)-1-({[(3Z,6S)-6-(5-chloro-2-methoxybenzyl)-3-(ethoxyimino)-7-oxo-1,4-diazepan-1-yl]carbonyl}amino)butyl]benzoic acid monoacetic acid solvate and optionally pharmaceutically acceptable carrier or excipient.

### EXAMPLES

While the disclosure has been described and illustrated with reference to certain particular embodiments thereof, those skilled in the art will appreciate that various adaptations, changes, modifications, substitutions, deletions, or additions of procedures and protocols may be made without departing from the spirit and scope of the disclosure. For example, effective dosages other than the particular dosages as set forth herein above may be applicable as a consequence of variations in responsiveness of the subject. The specific pharmacological responses observed may vary according to and depending upon the active compounds selected along with the present pharmaceutical carriers. Further, there responses may vary depending upon the type of formulation and mode of administration employed, and such expected variations or differences in the results are contemplated in accordance with the objects and practices of the present disclosure. It is intended, therefore, that the disclosure be defined by the scope of the claims which follow and that such claims be interpreted as broadly as is reasonable.

### Mouse Model

Mdr2 knock out (Mdr2 KO) mice are a genetic mouse model of PSC and display similar phenotypes to human PSC. Collagen content, ductular reaction and inflammation are elevated in Mdr2 knock out mice. Mdr2 KO mice also have enhanced mast cell (MC) presence. Total bile acid (TBA) content increases in Mdr2 knock out mice. Accumulation of chymase and tryptase positive mast cells (MC) in the liver was evaluated by immunohistochemistry. Metabolic parameters such as Total Bile Acids (serum and liver), Ductular Reaction (CK-19), Hepatic Fibrosis (Fast Green Sirius Red staining), Collagen deposition, Hydroxyproline levels, Fibrotic Gene Markers, cholangiocyte Activation (Desmin), inflammation and mast cell products, (F4/80, which is a macrophage marker) and Senescence and SASP markers (TGF-β1, p21, p16) are measured to determine the effect of compound 1 in mouse model of hepatobiliary disease.

### Example 1: Measuring the Efficacy of Test Compound in Treating Primary Sclerosing Cholangitis

### Object:

The purpose of this experiment was to evaluate the potential of test compounds of present disclosure in treating primary sclerosing cholangitis progression in a model of multi drug resistant 2 MDr2knockout (Mdr2 KO) mice. The model replicates the clinical features of PSC such as accumulation of bile salts and triglycerides in liver, hepatic steatosis, inflammation and fibrosis.

### Materials and Methods

Test Compound: compound 1 monoacetic acid solvate procured from Daiichi Sankyo.

Formulation of IP injection: compound 1 monoacetic acid solvate was suspended in 0.5% w/v Hydroxypropyl Cellulose

Animal: 10-12 weeks old Mdr2 homozygous knockout (Mdr2 KO) mice were raised on FVB/NJ background. All mice were maintained in a temperature-controlled environment (20° C. to 22° C.) with 12:12-hour light/dark cycles and fed standard chow with access to drinking water ad libitum.

### Experimental Procedure:

Administration of Drugs

10 and 20 mg/kg of compound 1 monoacetic acid solvate was administered intraperitoneally, daily, starting from 10 weeks of age to 12 weeks of age. Mdr2 knock out (Mdr2 KO) mice were randomized into different groups as indicated in Table 1.

TABLE 1
Dosing Groups

| Groups | Route | Dose | Dosing Regime | N |
|---|---|---|---|---|
| WT* | Intraperitoneal | Vehicle | 10 weeks to 12 | 8 |
| | | | weeks of age | |
| Mdr2 KO | Intraperitoneal | Vehicle | 10 weeks to 12 | 8 |
| mice | | | weeks of age | |
| Mdr2 knockout | Intraperitoneal | 10 mg/kg | 10 weeks to 12 | 8 |
| mice | | | weeks of age | |
| Mdr2 knockout | Intraperitoneal | 20 mg/kg | 10 weeks to 12 | 8 |
| mice | | | weeks of age | |

*WT—without treatment

### Assessment Procedures

### Reduction of Chymase and Tryptase Positive Mast Cells

The levels of chymase and tryptase positive cells was observed by measuring the reduction of expression of inflammatory markers such as mMCP1 and Tpbs2.

### Liver Bile Acid Accumulation

Liver and serum bile acid accumulation was evaluated by using a colorimetric assay from Crystal Chem Inc (cat #80471). For Liver, total bile acid (TBA) was extracted from homogenized liver. For serum, (TBAs) were measured in serum by enzyme immunoassay (EIA). 3-alpha-hydroxysteroid dehydrogenase and Nicotinamide adenine dinucleotide (NAD) converted TBA into 3-keto steroids and NADH. The NADH reacts with nitroblue tetrazolium (NBT) to form a dye and this is measured at 540 nm absorbance.

### Liver Inflammation and Fibrosis and Biliary Senescence

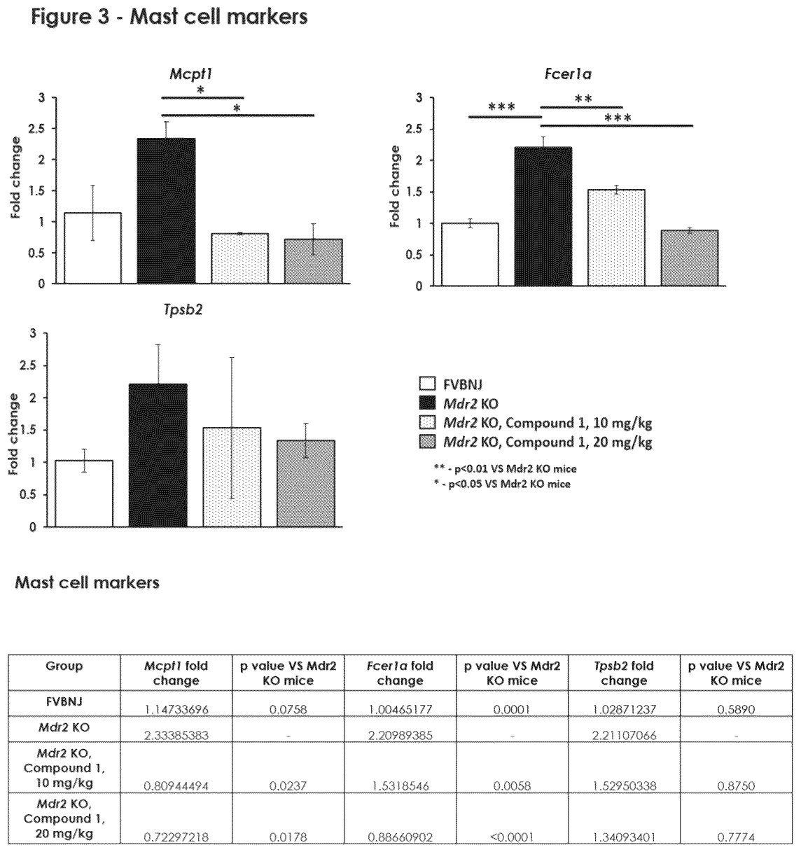

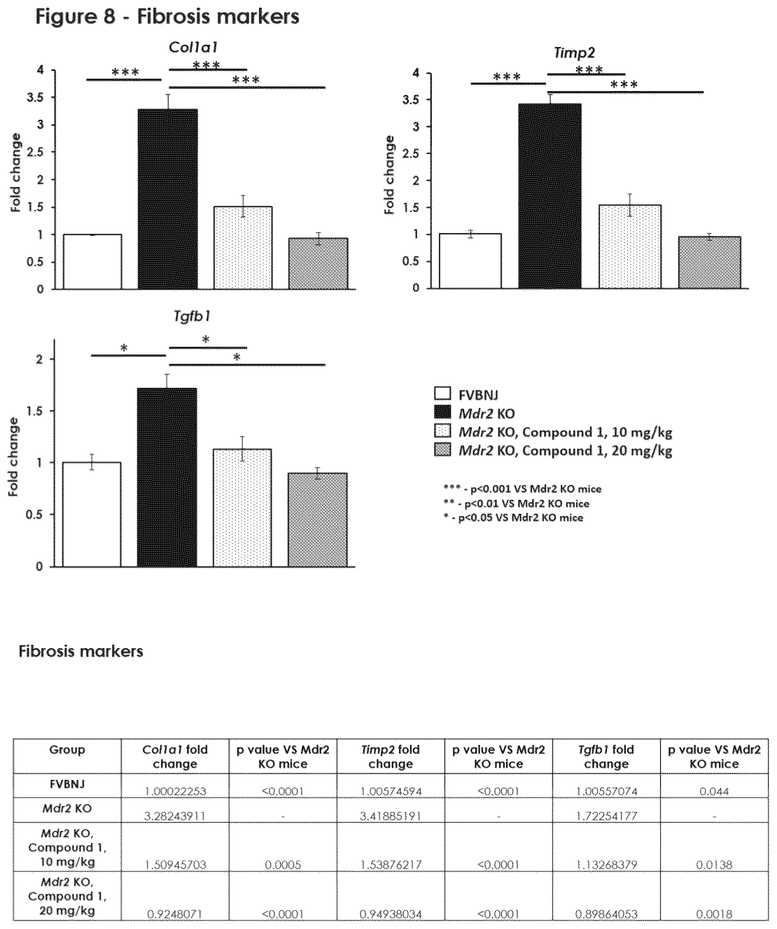

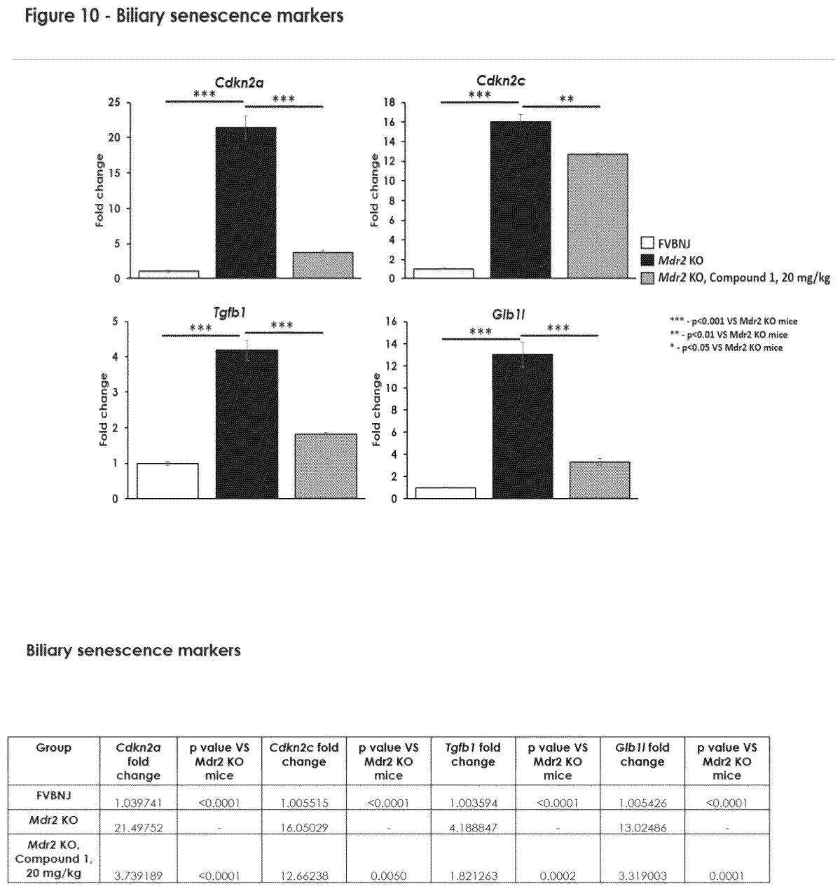

Liver inflammation, fibrosis and biliary senescence was evaluated in liver sections by quantitative real time PCR for mast cell markers such as Mcpt1, Fcer1a, Tpsb2 (See FIG. 3) and pro-fibrotic markers such as Col1a1, Timp2 and Tgfb1, (See FIG. 8)_biliary senescence markers such as Cdkn2a, Cdkn2c, Tgfb1 & Gl1bl. (See FIG. 10). Liver tissue was lysed, and total RNA was isolated using trizol and fold change was calculated using ΔΔCt method.

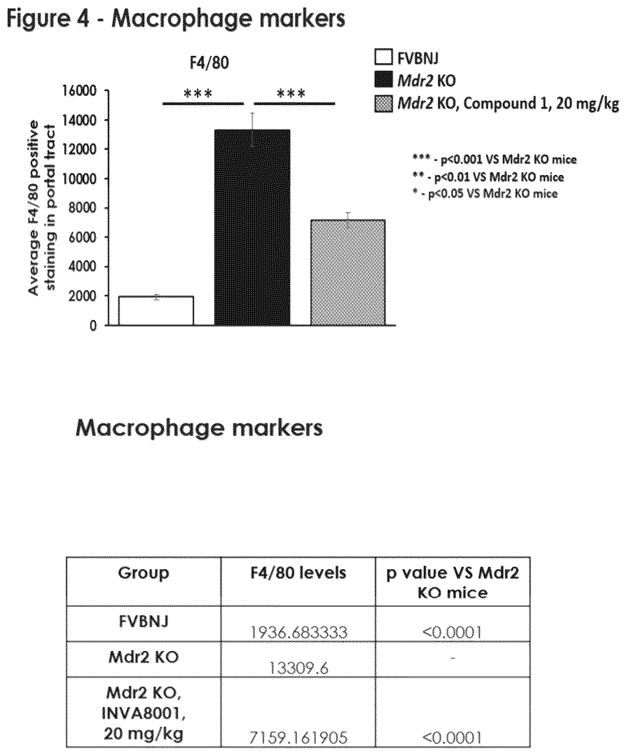

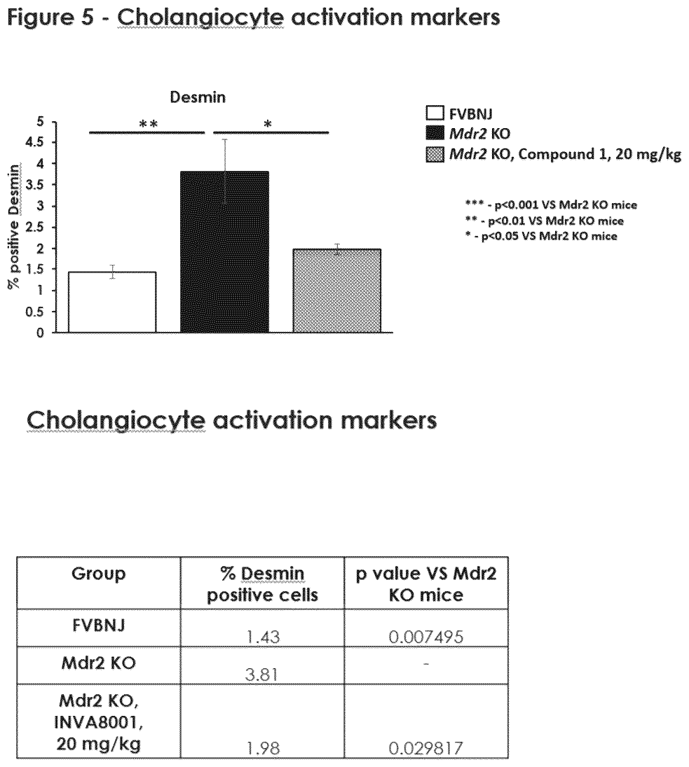

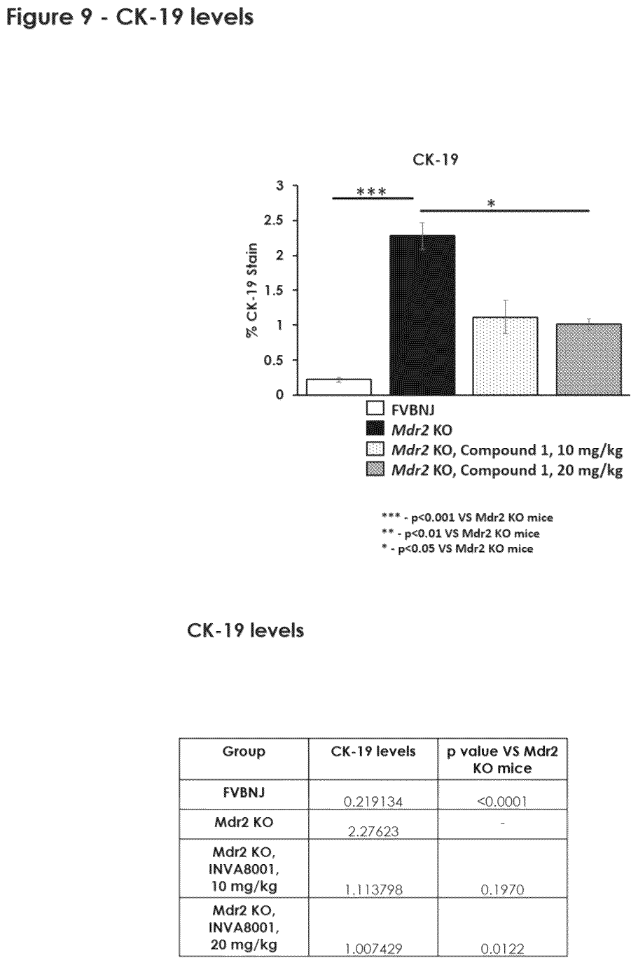

Fibrosis in liver was evaluated by quantitatively estimating collagen deposition with Fast green and Sirius red staining of formalin fixed paraffin-embedded liver sections (4 to 5 mm thick). Hepatic fibrosis and collagen deposition is evaluated by quantitative Fast Green/Sirius Red staining. Macrophage accumulation was determined by staining for F4/80 antibody. (See FIG. 4) Cholangiocyte activation was analyzed by immunofluorescence for Desmin (See FIG. 5) and CK19 (marker for cholangiocytes). (See FIG. 9)

### Ductular Reaction/Bile Duct Mass

The changes in intrahepatic bile duct mass (i.e., ductular reaction) and extent of cancer progression in liver was also assessed by quantitative immunohistochemistry in paraffin-embedded liver sections (4 to 5 mm thick) for CK19 (marker of proliferating ductal and lobular hepatocytes). (See FIG. 9)

### Statistical Analysis

All data is expressed as mean±SEM. Groups were analyzed by the student unpaired t test when two groups are analyzed or a two-way ANOVA when more than two groups are analyzed, followed by an appropriate post hoc test. p<0.05 was considered significant.

### Results

Treatment with Compound I in Mdr2 Knockout (Mdr2 KO) Mice Leads to Reduction in Accumulation of Chymase and Tryptase Positive Mast Cells (MC) in the Liver

### Reduction of Chymase and Tryptase Positive Mast Cells

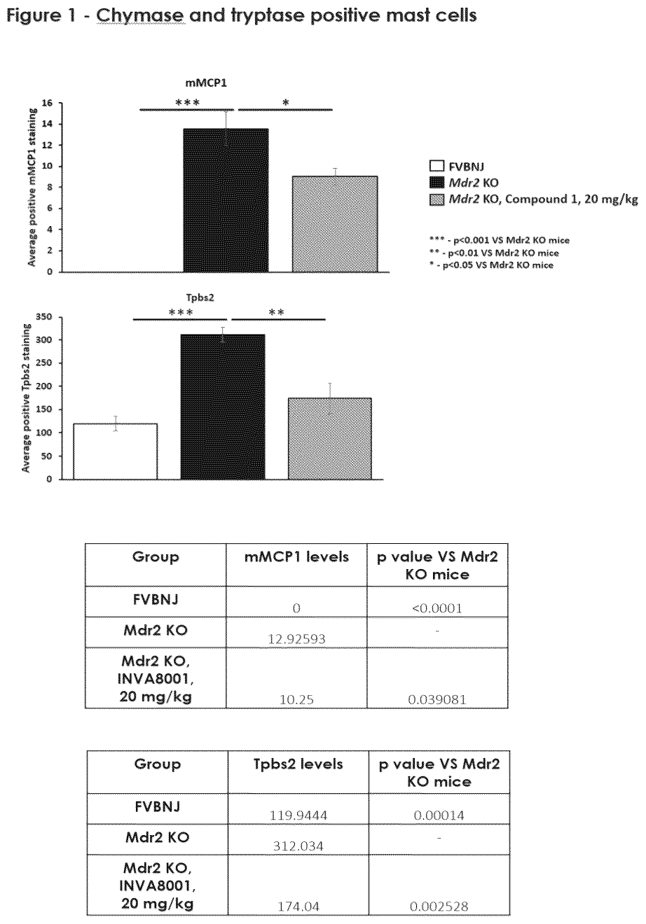

Compound 1 monoacetic acid solvate treatment reduced the levels of chymase and tryptase positive cells which was observed by reduction of expression of inflammatory markers such as mMCP1 and Tpbs2. (See FIG. 1) Mdr2 KO mice was treated with 20 mg/kg of Compound 1 monoacetic acid solvate and was compared with untreated Mdr2KO mice and FVBNJ wild type mice. The levels of both inflammatory markers (mMCP1 & Tpbs2) were significantly reduced indicating the reduction of chymase and tryptase positive cells.

### Liver Bile Acid Accumulation:

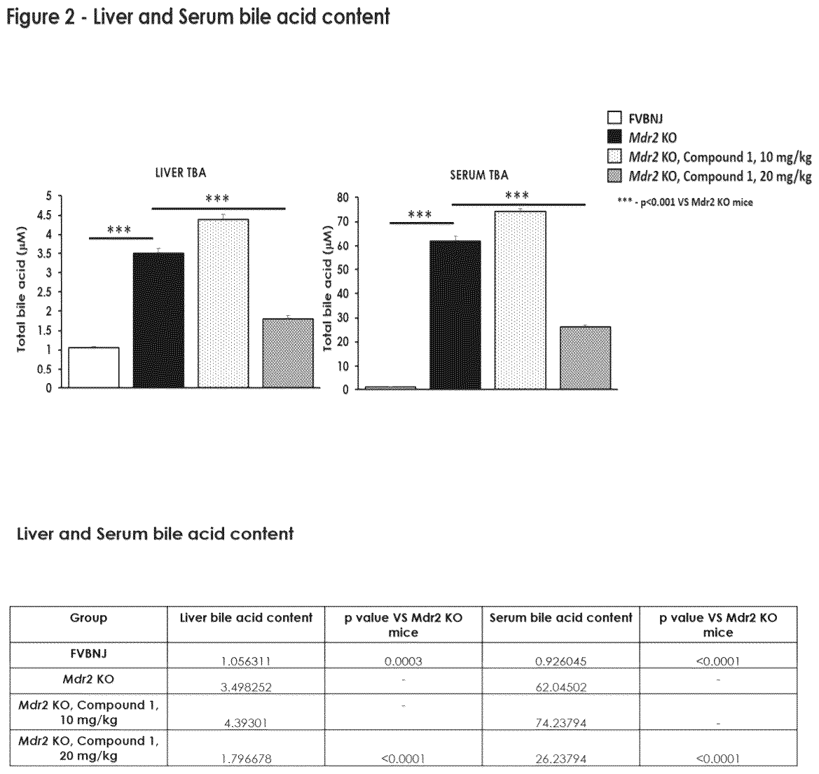

Liver total bile acid content (Mdr2 Knock Out=3.498252 μM, Mdr2 Knock Out, compound 1 monoacetic acid solvate (20 mg/kg)=1.796678, p<0.0001) and serum bile acid content (Mdr2 KO=62.04502 μM, Mdr2 KO, compound 1 monoacetic acid solvate (20 mg/kg)=26.23794, p<0.0001) was significantly decreased in Mdr2 knockout (Mdr2 KO) mice treated with 20 mg/kg compound 1 monoacetic acid solvate as shown in FIG. 2.

### Liver Inflammation, Fibrosis and Biliary Senescence

### Mast Cell Markers

Compound 1 monoacetic acid solvate treatment reduced the expression of inflammatory mast cell (MC) markers such as Mcpt1, (Mdr2 KO=2.33385383; Mdr2 KO, Compound 1 monoacetic acid solvate, 10 mg/kg=0.80944494, p=0.0237; Mdr2 KO Compound 1 monoacetic acid solvate, 20 mg/kg=0.72297218, p=0.0178), Fcer1 (Mdr2 KO=2.20989385; Mdr2 KO, Compound 1 monoacetic acid solvate, 10 mg/kg=1.5318546, p=0.0058; Mdr2 KO, compound 1 monoacetic acid solvate, 20 mg/kg=0.88660902, p=<0.0001), and Tpsb2 (Mdr2 KO=2.21107066; Mdr2 KO, compound 1 monoacetic acid solvate, 10 mg/kg=1.52950338, p=0.8750; Mdr2 KO, compound 1 monoacetic acid solvate, 20 mg/kg=1.34093401, p=0.7774) as shown in FIG. 3. The fold change, p values for each marker determined are provided in table at FIG. 3.

Likewise, treatment of Mdr2 knock out (Mdr2 KO) mice with compound 1 also reduces portal tract F4/80 immunoreactivity. (See FIG. 4). Treatment of Mdr2 knock out−/− mice with compound 1 also reduces portal tract F4/80 immunoreactivity. Mdr2 KO mice was treated with 20 mg/kg of Compound 1 monoacetic acid solvate and was compared with untreated Mdr2 KO mice and FVBNJ wild type mice.). The p values and F4/80 levels determined are provided in table at FIG. 4.

### Cholangiocyte Activation

Cholangiocyte activation was analyzed by observing immunofluorescence for Desmin (See FIG. 5) and CK19 (marker for cholangiocytes). (See FIG. 9). Treatment of Mdr2 Knock out mice with compound 1 shows reduction of expression of desmin levels when compared with untreated Mdr2 KO mice.). The p values and % of Desmin positive cells are provided in table at FIG. 5.

### Collagen Deposition and Hydroxy Proline Levels

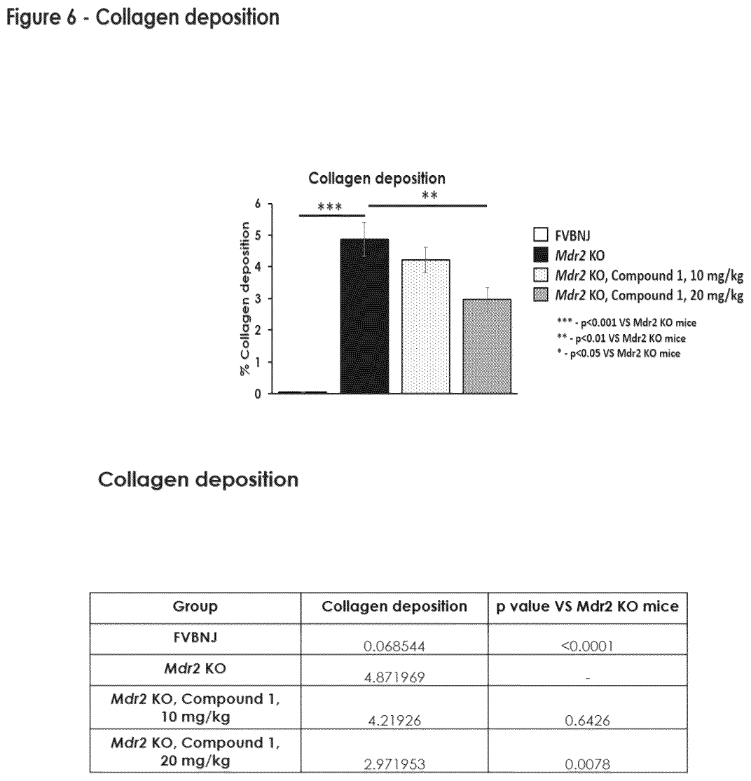

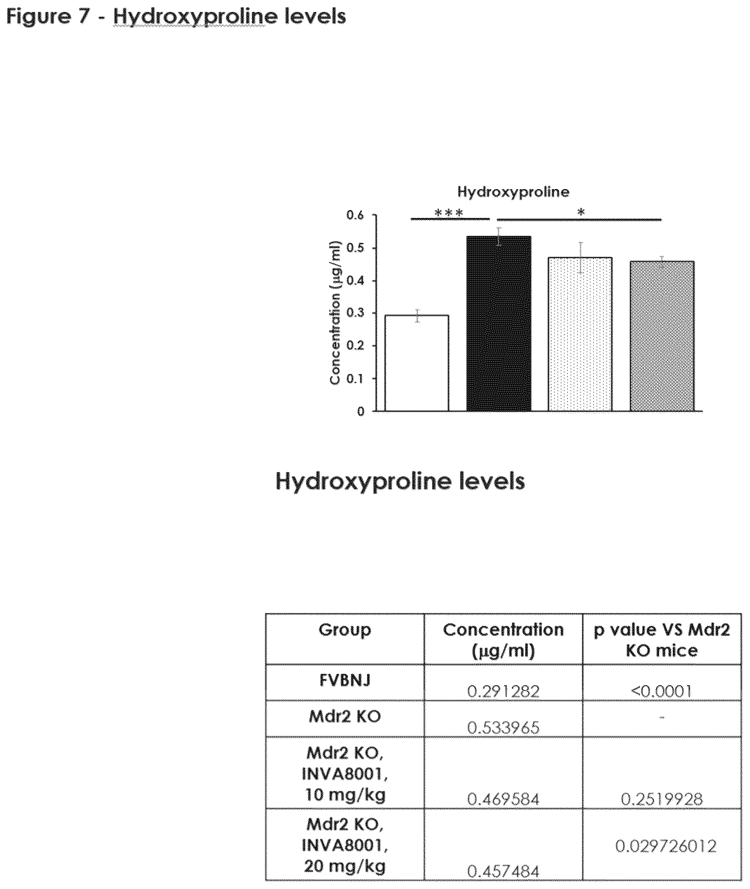

Compound 1 monoacetic acid solvate treatment restricted collagen deposition and hydroxyproline accumulation (Mdr2 KO=4.871969; Mdr2 KO, compound 1 monoacetic acid solvate, 10 mg/kg=4.21926, p=0.6426; Mdr2 KO, compound 1 monoacetic acid solvate, 20 mg/kg=2.971953, p=0.0078) as shown in FIGS. 6-7. The p values and the amount of collagen deposition determined are provided in table at FIG. 6. The p values and hydroxyproline levels determined are provided in table at FIG. 7.

### Fibrosis

Compound 1 monoacetic acid solvate treatment reduced the expression of pro-fibrotic fibrosis markers such as Col1a1 (Mdr2 KO=3.28243911; Mdr2 KO, compound 1 monoacetic acid solvate, 10 mg/kg=1.50945703, p=0.0005; Mdr2 KO, compound 1 monoacetic acid solvate, 20 mg/kg=0.9248071, p<0.0001), Timp2 (Mdr2 KO=3.41885191; Mdr2 KO, compound 1 monoacetic acid solvate, 10 mg/kg=1.53876217, p<0.0001; Mdr2 KO, compound 1 monoacetic acid solvate, 20 mg/kg=0.94938034, p<0.0001) and Tgfb1 (Mdr2 KO=1.72254177; Mdr2 KO, compound 1 monoacetic acid solvate, 10 mg/kg=1.13268379, p=0.0138; Mdr2 KO, compound 1 monoacetic acid solvate, 20 mg/kg=0.89864053, p=0.0018) in Mdr2 knockout (Mdr2 KO) mice as shown in FIG. 8. The fold change, p values for each marker determined are provided in table at FIG. 8.

### Ductular Reaction/Increase in Bile Duct Mass

Assessed by immunohistochemistry, compound 1 monoacetic acid solvate treatment reduced the number of proliferating ductal and lobular hepatocytes (CK 19, a marker of cancer progression also). (Mdr2 KO=2.27623 μM; Mdr2 KO, compound 1 monoacetic acid solvate, 10 mg/kg=1.113798 μM, p=0.1970; Mdr2 KO, compound 1 monoacetic acid solvate, 20 mg/kg=1.007429, p=0.0122) as shown in FIG. 9). The p values and Ck-19 levels determined are provided in table at FIG. 9.

### Biliary Senescence:

Compound 1 monoacetic acid solvate (20 mg/kg) administration restricted the expression of senescence markers Cdkn2a (Mdr2 KO=21.49752; Mdr2 KO, compound 1 monoacetic acid solvate, 20 mg/kg=3.739189, p<0.0001), Cdkn2c (Mdr2 KO=16.05029; Mdr2 KO, compound 1 monoacetic acid solvate, 20 mg/kg=12.66238, p=0.0050), Tgfb1 (Mdr2 KO=4.188847; Mdr2 KO, compound 1 monoacetic acid solvate, 20 mg/kg=1.821263, p=0.0002), Glb1l (Mdr2 KO=13.02486; Mdr2 KO, compound 1 monoacetic acid solvate, 20 mg/kg=3.319003, p=0.0001) in Mdr2 knockout mice (Mdr2 KO) as shown in FIG. 10. The fold change, p values for each marker determined are provided in table at FIG. 10.

### Conclusion:

This data proved that administration of compound 1 monoacetic acid solvate to a PSC animal model significantly reduced the expression of inflammatory mast cell (MC) and macrophage accumulation, limited cholangiocyte activation, reduced the expression of pro-fibrotic genes s, restricted collagen and hydroxyproline deposition, reduced the number of proliferating ductal and lobular hepatocytes and restricted the expression of senescence markers. Administration of compound 1 ameliorated ductular reaction (DR), hepatic damage, senescence/SASP, fibrosis along with chymase-positive mast cell (MC) infiltration and activation in Mdr2 knock out (Mdr2 KO) mice. Inhibition of chymase by compound 1 monoacetic acid solvate targets PSC phenotypes and ameliorates pathogenesis by decreased biliary senescence and Senescence-Associated Secretory Phenotype (SASP) factors thereby providing therapeutic benefit to subjects with liver diseases wherein the MCs promote pathogenesis.

Human PSC and Mdr2 knock out (Mdr2 KO) mice display enhanced ductular reaction and biliary senescence. Without being bound by theory, it is believed that senescence triggers cholangiocytes into a senescent-associated secretory phenotype (SASP) increasing factors like TGF-b1. Increased biliary senescence/SASP induces mast cell (MC) recruitment in PSC progression

Administration of Compound 1 results in decreased cholangiocyte proliferation, reduced angiogenesis and inflammation, inhibition of TGF-b and collagen-mediated liver fibrosis and decreased HSC activation.

Chymase positive mast cells (MC) are significantly upregulated in human PSC and in Mdr2 knock out (Mdr2 KO) mice. Treatment with the specific chymase inhibitor, Compound 1 decreases MC presence and MC marker gene expression. In Mdr2 knock out (Mdr2 KO) mice treated with Compound 1, PSC phenotypes are ameliorated including liver damage, ductular reaction, TGF-b1, biliary senescence, hepatic fibrosis and portal inflammation. Thus a 14-day treatment with Compound 1 improves PSC-associated phenotypes in Mdr2 knock out (Mdr2 KO) mice.

### Example 2: Measuring the Efficacy of Test Compound in Treating Ulcerative Colitis

The purpose of this experiment was to evaluate the potential of test compounds of the present disclosure in treating Ulcerative colitis (UC) in a model of DSS-induced colitis. Dextran sulfate sodium (DSS) is a sulfated polysaccharide with variable molecular weights. Administration of DSS in mice causes human ulcerative colitis-like pathologies due to its toxicity to colonic epithelial cells, which results in compromised mucosal barrier function. DSS-induced colitis is a well-studied animal model for ulcerative colitis. The model replicates the clinical features of UC such as weight loss, diarrhea and occult blood in stool etc.

### Materials and Methods

Test Compound: compound 1 monoacetic acid solvate procured from Daiichi Sankyo. Formulation of IP injection: compound 1 monoacetic acid solvate was suspended in 0.5% w/v Hydroxypropyl Cellulose

Animal: For the DSS-induced acute model of experimental colitis, 8-week-old C57Bl/6N mice (weight between 17 and 20 grams, all females from Charles River Laboratories) were used. All mice were maintained in a temperature-controlled environment (20° C.±4° C.) with 12:12-hour light/dark cycles, relative humidity was maintained at 55%±10%, ventilation with 8-10 air changes per hour and fed standard chow with access to drinking water ad libitum.

The experimental protocols for handling mice were submitted and approved by the Italian Ministry of Health (IACUC #1174; Authorization no: 1144/2020-PR (prot. DC8BD.226)). Regular use of environmental enrichment has been made in order to decrease stress in animals. To obtain an environment respectful of mice physiological needs, the following parameters have been observed.

Animal handling was reduced to the minimum necessary to reduce stress. For the proposed models, no consequences for other organs were described in the literature except for the colon. Mice were randomly grouped and randomly allocated in groups of four mice in standard rectangular cages, 1 week prior to the start of the study.

Mice were treated with one oral cycles of 2.5% DSS (weight/volume) (DSS, molecular mass, 40 kDa; MP Biomedicals), characterized by 7 days of DSS exposure in drinking water, followed by 3 days of regular drinking water.

### Experimental Procedure

Administration of drugs: 3 days after starting DSS administration, mice were treated with testing drugs up to day 9, according to the experimental groups and the dose regimen described in table below. 0.5% (w/v) Hydroxypropyl cellulose was used as the vehicle.

TABLE 2
Dosing Groups

| Groups | Route | Dose | Dosing Regime | N |
|---|---|---|---|---|
| Sham control | Intraperitoneal | Vehicle | Day 3 to 9, QD | 10 |
| DSS + vehicle IP | Intraperitoneal | Vehicle | Day 3 to 9, QD | 10 |
| DSS + | Intraperitoneal | 20 mg/kg | Day 3 to 9, QD | 10 |
| Compound 1 | | | | |
| DSS + anti-TNF | Intraperitoneal | 15 mg/kg | Day 3 to 9, QD | 10 |

Induction of Colitis

From day 0 to 7, the animals received DSS in drinking water and the drugs were administered from day 3 to 9. Body weight and clinical observations such as diarrhea and bleeding from rectum were recorded daily post colitis induction until day 10.

Assessment Procedures

Disease activity Index (DAI) and body weight loss were measured. Colitis severity was calculated using a disease activity index (DAI) score based on daily evaluation of body weight, stool consistency, and presence of blood in the stools, as determined using Hemoccult SENSA Cards (Beckman Coulter).

Grading of intestinal inflammation was determined according to the criteria proposed by Cooper et al (Cooper H S et al., Clinicopathologic study of dextran sulfate sodium experimental murine colitis. Lab Invest. 1993 August; 69(2):238-49, the DAI has been determined by scoring changes in variety of factors such as weight loss, stool consistency, and occult bleeding. The weight loss score ranges from 0 to 4, where zero signals no weight loss, score of 1 indicates the presence of 1-5% weight loss, a score of 2 signals the presence of 5-10% weight loss, a score of 3 signals the presence of 10-20% weight loss and a score of 4 represents greater than 20% weight loss. Likewise, the stool consistency score ranges from 0 to 4 with a score of zero representing normal stools, a score of 2 represents loose stools and a score of 4 represents diarrhea. The rectal bleeding is observed by scores ranging from 0 to 4, where a score of zero represents normal or no significant bleeding, a score of 2 indicates the presence of occult bleeding and a score of 4 indicates the presence of gross bleeding. A 5-point (0-4) DAI was obtained by calculating an average of body weight score, stool consistency and rectal bleeding.

### Results

Intraperitoneal treatment with Compound 1 in DSS induced colitis model significantly attenuated pathological symptoms of DSS-induced colitis such as body weight loss, diarrhea, rectal bleeding and DAI score with comparable efficiency to anti-TNF antibodies. The results indicate that Compound 1 monoacetic acid solvate is as efficient as anti-TNF antibodies for treating UC.

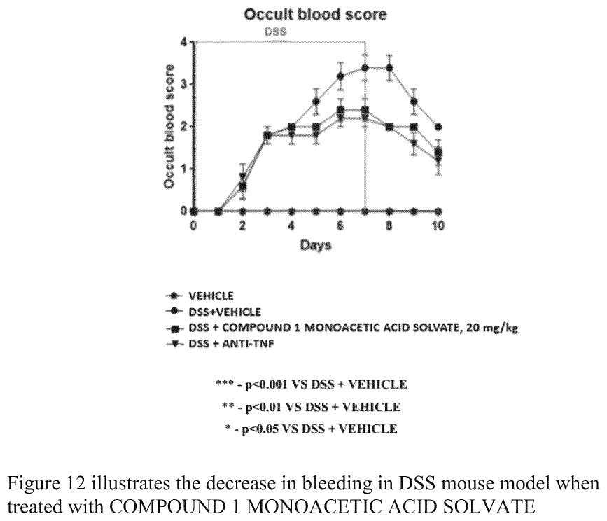

The Body weight loss graph is shown in FIG. 12. Mice not exposed to DSS did not have a body weight loss during the observed period of 0-10 days. Mice exposed to DSS developed colitis and exhibited loss in body weight. DSS exposed mice treated with compound 1 monoacetic acid solvate showed reduction in the body weight loss and were comparable to improvement seen in DSS exposed mice treated with TNF.

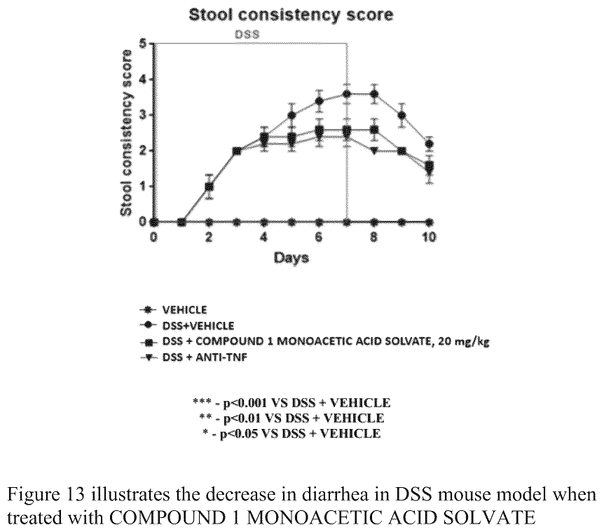

The Occult blood score graph is shown in FIG. 13. Mice not exposed to DSS did not have measurable blood loss during the observed period of 0-10 days. Mice exposed to DSS developed colitis and exhibited significant bleeding. DSS exposed mice treated with compound 1 monoacetic acid solvate showed reduction in bleeding and were comparable to improvement seen in DSS exposed mice treated with TNF. Administration of compound 1 significantly inhibits colon length reduction and improved the colon weight/length ratio.

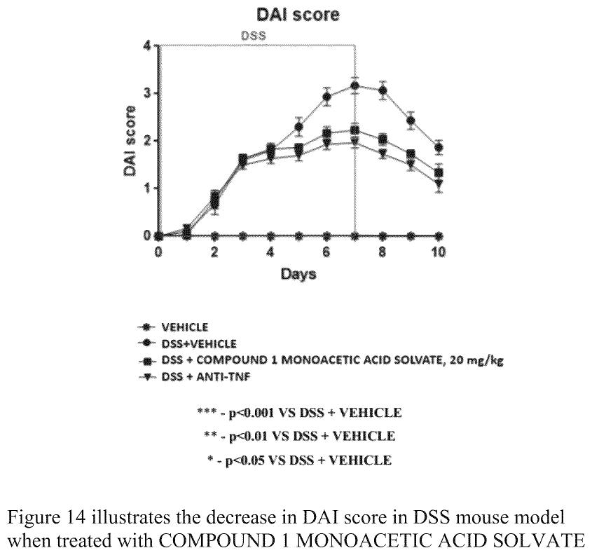

The Stool consistency score graph is shown in FIG. 14. Mice not exposed to DSS did not have diarrhea during the observed period of 0-10 days. Mice exposed to DSS developed colitis and exhibited significant diarrhea and loose stools. DSS exposed mice treated with compound 1 monoacetic acid solvate showed reduction in diarrhea and were comparable to improvement seen in DSS exposed mice treated with TNF.

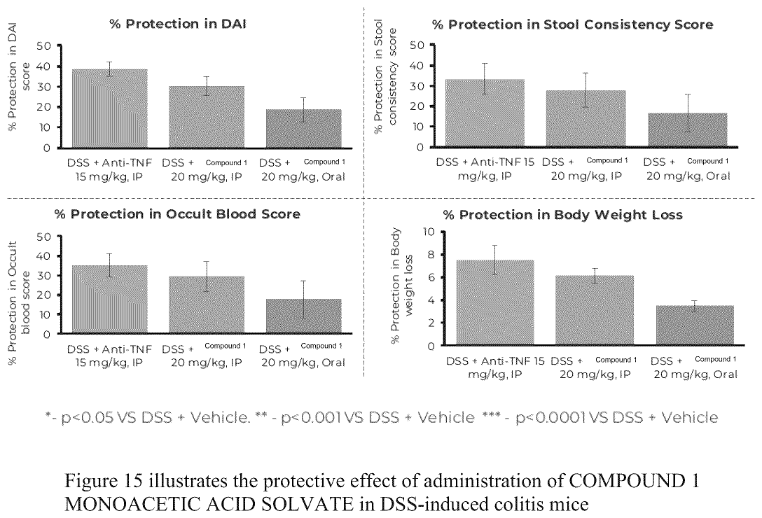

The DAI score graph is shown in FIG. 15. Mice not exposed to DSS had a low DAI score of zero during the observed period of 0-10 days. Mice exposed to DSS developed colitis and exhibited significant DAI scores of 3 and above. DSS exposed mice treated with compound 1 showed reduction in DAI scores (<2). FIG. 16 the protective effect of administration of compound 1 in DSS-induced colitis mice.

### Conclusion:

This data shows that administration of compound 1 monoacetic acid solvate to DSS induced colitis model significantly reduced body weight loss, reduced blood loss, reduced diarrhea and reduced DAI score indicating significant improvement of colitis condition. These results showed that compound 1 has a protective effect against DSS-induced acute colitis, indicating that compound 1 can serve as an effective treatment for IBD.

### Other Embodiments

While specific embodiments of the subject matter have been discussed, the above specification is illustrative and not restrictive. Many variations will become apparent to those skilled in the art upon review of this specification and the claims below.

Recitation of a listing of elements in any definition of a variable herein includes definitions of that variable as any single element or combination (or sub combination) of listed elements. Recitation of an embodiment herein includes that embodiment as any single embodiment or in combination with any other embodiments or portions thereof. The full scope of the invention should be determined by reference to the claims, along with their full scope of equivalents, and the specification, along with such variations.

What is claimed is:

1. A method for treating hepatobiliary disease in a subject in need thereof, said method comprising administering to the subject a therapeutically effective amount of 2-amino-4-[(1R-1-({[(3Z,6S)-6-(5-chloro-2-methyoxybenzyl)-3-(ethoxyimino)-7-oxo-1,4-diazepan-1-yl]carbonyl}amino)butyl]benzoic acid (compound 1) or a pharmaceutically acceptable salt or solvate thereof.

2. The method of claim 1, wherein said administration of said therapeutically effective amount of said compound 1 or a pharmaceutically acceptable salt or solvate thereof ameliorates one or more of infiltration of at least one of mast cells or macrophages, bile acid accumulation, cholangiocyte proliferation, biliary senescence, fibrosis, collagen deposition in said subject suffering from hepatobiliary disease.

3. The method of claim 1, wherein said administration of said therapeutically effective amount of said compound 1 or a pharmaceutically acceptable salt or solvate thereof ameliorates one or more of occult blood in stool, fecal incontinence, fever, fatigue, weight loss, mast cell and eosinophil counts, dysphagia, gastroesophageal reflux disease (GERD), bloating, early satiety, loss of appetite, jaundice, pruritis, hepatomegaly, cirrhosis, biliary obstruction, abdominal pain, nausea, diarrhea, Ulcerative colitis, Crohn's disease, Irritable bowel syndrome (IBS), Proctitis, Celiac disease, ileitis, duodenitis, diverticulitis, spasms, and inflammation in said subject suffering from hepatobiliary disease.

4. The method of claim 1, wherein the compound 1 or a pharmaceutically acceptable salt or solvate thereof is a selective for chymase over cathepsin G.

5. The method of claim 1, wherein the hepatobiliary disease is selected from the group consisting of acute hepatitis, alcoholic hepatitis, alcoholic liver disease, extrahepatic cholestasis, hepatic fibrosis, liver abscess, liver cirrhosis, colitis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, non-alcoholic steatohepatitis, polycystic liver disease, veno-occlusive disease, and Wilson's disease.

6. The method of claim 1, wherein said therapeutically effective amount of said compound 1 or a pharmaceutically acceptable salt or solvate thereof is from about 20 mg to about 400 mg per unit dose.

7. The method of claim 1 wherein said compound 1 is:

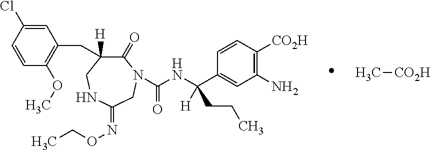

8. The method of claim 1, wherein said method for treating comprises preventing hepatobiliary disease in said subject.

9. The method according to claim 1, wherein said therapeutically effective amount of said compound 1 or a pharmaceutically acceptable salt or solvate thereof is administered orally to the subject once a day, twice a day, thrice a day, once in two days, once in three days, or once a week.

10. A method for treating primary sclerosing cholangitis in a subject in need thereof, said method comprising administering to said subject a therapeutically effective amount of 2-amino-4-[(1R-1-({[(3Z,6S)-6(5-chloro-2-methyoxybenzyl)-3-(ethoxyimino)-7-oxo-1,4-diazepan-1-yl]carbonyl}amino)butyl]benzoic acid (compound 1) or a pharmaceutically acceptable salt or solvate thereof.

11. The method of claim 10, wherein said administering of said therapeutically effective amount of said compound 1 or a pharmaceutically acceptable salt or solvate thereof reduces one or more of infiltration of at least one of mast cells or macrophages, bile acid accumulation, cholangiocyte proliferation, biliary senescence, fibrosis, and collagen deposition in said subject suffering from primary sclerosing cholangitis.

12. The method of claim 10, wherein said administration of said therapeutically effective amount of said compound 1 ameliorates one or more of occult blood in stool, fecal incontinence, fever, fatigue, weight loss, mast cell and eosinophil counts, dysphagia, gastroesophageal reflux disease (GERD), bloating, early satiety, loss of appetite, jaundice, pruritis, hepatomegaly, cirrhosis, biliary obstruction, abdominal pain, nausea, diarrhea, Ulcerative colitis, Crohn's disease, Irritable bowel syndrome (IBS), Proctitis, Celiac disease, ileitis, duodenitis, diverticulitis, spasms, and inflammation in said subject suffering from primary sclerosing cholangitis.

13. The method of claim 10, wherein said administering of said therapeutically effective amount of said compound 1 or a pharmaceutically acceptable salt or solvate thereof treats at least one of inflammatory bowel disease and colitis in said subject.

14. The method of claim 10, wherein said therapeutically effective amount of said compound 1 or a pharmaceutically acceptable salt or solvate thereof is from about 20 mg to about 400 mg per unit dose.

15. The method of claim 10 wherein said compound 1 is:

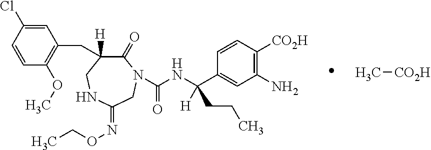

16. The method of claim 10, wherein said method for treating comprises preventing primary sclerosing cholangitis in said subject in need thereof.

17. The method according to claim 10, wherein said therapeutically effective amount of said compound or a pharmaceutically acceptable salt or solvate thereof is administered orally once a day, twice a day, thrice a day, once in two days, or once in three days or once a week.

18. A method for treating primary biliary cholangitis in a subject in need thereof, said method comprising administering to the subject a therapeutically effective amount of 2-amino-4-[(1R-1-({[(3Z,6S)-6(5-chloro-2-methyoxybenzyl)-3-(ethoxyimino)-7-oxo-1,4-diazepan-1-yl]carbonyl}amino)butyl]benzoic acid (compound 1) or a pharmaceutically acceptable salt or solvate thereof.

19. The method of claim 18, wherein said administering of said therapeutically effective amount of said compound 1 or a pharmaceutically acceptable salt or solvate thereof reduces infiltration of at least one of mast cells or macrophages, bile acid accumulation, cholangiocyte proliferation, biliary senescence, fibrosis, and collagen deposition in said subject suffering from primary biliary cholangitis.

20. The method of claim 18, wherein said administering of said therapeutically effective amount of said compound 1 ameliorates one or more of occult blood in stool, fecal incontinence, fever, fatigue, weight loss, mast cell and eosinophil counts, dysphagia, gastroesophageal reflux disease (GERD), bloating, early satiety, loss of appetite, jaundice, pruritis, hepatomegaly, cirrhosis, biliary obstruction, abdominal pain, nausea, diarrhea, Ulcerative colitis, Crohn's disease, Irritable bowel syndrome (IBS), Proctitis, Celiac disease, ileitis, duodenitis, diverticulitis, spasms, and inflammation in said subject suffering from primary biliary cholangitis.

21. The method of claim 18, wherein said administering of said therapeutically effective amount of said compound 1 or a pharmaceutically acceptable salt or solvate thereof treats at least one of inflammatory bowel disease and colitis in said subject.

22. The method of claim 18, wherein said therapeutically effective amount of said compound 1 or a pharmaceutically acceptable salt or solvate thereof is from about 20 mg to about 400 mg per unit dose.

23. The method of claim 18 wherein said compound 1 is:

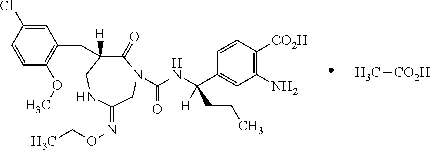

24. The method of claim 18, wherein said method for treating comprises preventing primary biliary cholangitis in said subject.

25. The method according to claim 18, wherein said therapeutically effective amount of said compound 1 or a pharmaceutically acceptable salt or solvate thereof is administered orally once a day, twice a day, thrice a day, once in two days, or once in three days or once a week.

26. The method according to claim 25, wherein the therapeutic effective amount of compound 1 or a pharmaceutically acceptable salt or solvate thereof are administered in unit dosage form as a pellet, multiunit particulate system (MUPS), tablets, troches, lozenges, dispersible powders or granules, or a hard or soft capsule, powders, sustained-release formulations, microcapsules.

27. A method for treating at least one of primary sclerosing cholangitis and primary biliary cholangitis in a subject in need thereof, said method comprising administering to the subject a therapeutically effective amount of a compound of Formula (I), or a pharmaceutically acceptable salt or solvate thereof: 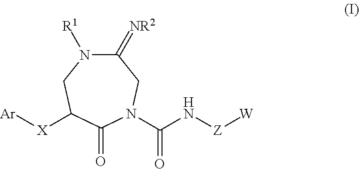
wherein,
Ar is (1) a C6 to C14 aromatic hydrocarbon group, (2) a 5 to 8-membered aromatic heterocyclic group having 1 to 4 hetero atoms selected from the group consisting of a nitrogen atom, sulfur atom, and oxygen atom, in place of a carbon atom, or (3) a bicyclic or tricyclic aromatic group formed by condensation of the above aromatic heterocyclic group and a C6 to C14 aromatic hydrocarbon ring,
where the above groups (1) to (3) of Ar are optionally substituted with 1 to 5 groups selected from the group consisting of:
(viii) a halogen atom,
(ix) nitro,
(x) cyano,
(xi) C1 to C6 alkyl optionally substituted with 1 to 3 halogen atoms,
(xii) C2 to C6 alkenyl optionally substituted with 1 to 3 halogen atoms,
(xiii) C2 to C6 alkynyl optionally substituted with 1 to 3 halogen atoms,
(xiv) C3 to C6 cycloalkyl,
(ix) hydroxyl,
(xiii) C1 to C6 alkoxy optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(xiv) C1 to C5 alkylenedioxy,
(xv) C1 to C6 alkylthio optionally substituted with 1 to 3 groups selected from the group consisting of a halogen atom, mono- or di-C1 to C6 alkylamino, C1 to C6 alkoxy, mono- or di-C1 to C6 alkylcarbamoyl, mono- or di-C7 to C16 aralkylcarbamoyl, mono- or di-C1 to C10 heteroaryl-C1 to C6 alkylcarbamoyl, carboxyl, and C1 to C6 alkoxycarbonyl, or substituted with 1 to 13 deuterium atoms,
(xvi) amino,
(xix) mono-C1 to C6 alkylamino,
(xx) di-C1 to C6 alkylamino,
(xxi) 5- to 6-membered cyclic amino,
(xxii) C1 to C6 alkylcarbonyl,
(xxiii)carboxyl,
(xxiv) C1 to C6 alkoxycarbonyl,
(xxviii)carbamoyl,
(xxix) thiocarbamoyl,
(xxx) mono-C1 to C6 alkylcarbamoyl,
(xxxi) di-C1 to C6 alkylcarbamoyl,
(xxxii) 5- to 6-membered cyclic aminocarbonyl,
(xxxiii)sulfo,
(xxxiv) C1 to C6 alkylsulfonyl,
(xxxv) C1 to C6 alkoxycarbonylamino,
(xxxvi) C1 to C6 alkylcarbonylamino,
(xxxi) mono- or di-C1 to C6 alkylaminocarbonylamino,
(xxxii) aminosulfonyl, and
(xxxiii) mono- or di-C1 to C6 alkylaminosulfonyl, or
substituted with 1 to 9 deuterium atoms,
X is (1) a connecting bond, (2) linear or branched C1 to C6 alkylene optionally substituted with 1 to 12 deuterium atoms, (3) an oxygen atom, (4) NR³, where R³ is a hydrogen atom or a C1 to C6 alkyl group, or (5) —S(O)m-, where m is an integer of 0 to 2,
Z is (1) a connecting bond or (2) CR⁴R⁵ where R⁴ and R⁵ are, independently,
(G) a hydrogen atom,
(H) a deuterium atom,
(I) C1 to C6 alkyl optionally substituted with 1 to 5 groups selected from the group consisting of (i) carboxyl, (ii) C1 to C6 alkoxycarbonyl, (iii) phenyl, (iv) hydroxyl, (v) C1 to C6 alkoxy, (vi) halogen atom, and (vii) C3 to C6 cycloalkyl, or substituted with 1 to 13 deuterium atoms,
(J) C3 to C6 cycloalkyl optionally substituted with 1 to 5 groups selected from the group consisting of (i) a halogen atom and (ii) an alkyl group optionally substituted with 1 to 3 halogen atoms or optionally substituted, with 1 to 11 deuterium atoms,
(K) COOR⁶, where R⁶ is a hydrogen atom or C1 to C6 alkyl, or
(L) CONR⁷R⁸, where R⁷ and R⁸ are, independently,
(d) hydrogen atom,
(e) C1 to C6 alkyl optionally substituted with 1 to 3 groups selected from the group consisting of (i) a halogen atom, (ii) C3 to C6 cycloalkyl, (iii) carboxyl, (iv) C1 to C6 alkoxycarbonyl, (v) C1 to C6 alkylcarbonyl, (vi) carbamoyl, (vii) mono-C1 to C6 alkylcarbamoyl, (viii) di-C1 to C6 alkylcarbamoyl, (ix) C6 to C12 aryl, and (x) C1 to C10 heteroaryl,
(f) C6 to C14 aromatic hydrocarbon group,
(f) a 5- to 8-membered aromatic heterocyclic group having 1 to 4 hetero atoms selected from the group consisting of a nitrogen atom, sulfur atom, and oxygen atom, in place of a carbon atom, or
(g) a bicyclic or tricyclic aromatic group formed by condensation of the above aromatic heterocyclic, group and a C6 to C14 aromatic hydrocarbon ring,
where each of the groups (c) to (e) is optionally substituted with 1 to 5 groups selected from the group consisting of (i) a halogen atom, (ii) nitro, (iii) cyano, (iv) C1 to C6 alkyl optionally substituted with 1 to 3 halogen atoms, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 halogen atoms, (x) C1 to C5 alkylenedioxy, (xi) C1 to C6 alkylthio optionally substituted with 1 to 3 halogen atoms, (xii) amino, (xiii) mono-C1 to C6 alkylamino, (xiv) di-C1 to C6 alkylamino, (xv) 5- to 6-membered cyclic amino, (xvi) C1 to C6 alkylcarbonyl, (xvii) carboxyl, (xviii) C1 to C6 alkoxycarbonyl, (xix) carbamoyl, (xx) thiocarbamoyl, (xxi) mono-C1 to C6 alkylcarbamoyl, (xxii) di-C1 to C6 alkylcarbamoyl, (xxiii) C6 to C10 arylcarbamoyl, (xxiv) C1 to C10 heteroarylcarbamoyl, (xxv) sulfo, (xxvi) C1 to C6 alkylsulfonyl, (xxvii) aminosulfonyl, and (xxviii) mono- or di-C1 to C6 alkylaminosulfonylamino, or substituted with 1 to 9 deuterium atoms,
W is (1) a hydrogen atom, (2) a C6 to C14 aromatic hydrocarbon group, (3) a 5- to 8-membered aromatic heterocyclic group having 1 to 4 hetero atoms selected from a nitrogen atom, a sulfur atom, and an oxygen atom, in place of a carbon atom, (4) a bicyclic, or tricyclic aromatic group formed by condensation of the above aromatic heterocyclic group and a C6 to C14 aromatic hydrocarbon ring, or a deuterium atom,
where, each of the groups (2) to (4) of the above W is optionally substituted with 1 to 5 groups selected from the group consisting of (i) a halogen atom, (ii) nitro, (iii) cyano, (iv) C1 to C6 alkyl optionally substituted with 1 to 3 groups selected from the group consisting of a halogen atom, amino, C1 to C6 alkoxycarbonyl, C1 to C6 alkoxycarbonylamino, and carboxyl, (v) C2 to C6 alkenyl optionally substituted with 1 to 3 halogen atoms, (vi) C2 to C6 alkynyl optionally substituted with 1 to 3 halogen atoms, (vii) C3 to C6 cycloalkyl, (viii) hydroxyl, (ix) C1 to C6 alkoxy optionally substituted with 1 to 3 groups selected from the group consisting of a halogen atom, hydroxyl, C1 to C6 alkoxy, amino and mono- or di-C1 to C6 alkylamino, (x) C1 to C5 alkylenedioxy, (xi) C1 to C6 alkylthio optionally substituted with 1 to 3 groups selected from the group consisting of a halogen atom, hydroxyl, C1 to C6 alkoxy, amino, and mono- or di-C1 to C6 alkylamino, (xii) amino, (xiii) mono-C1 to C6 alkylamino, (xiv) di-C1 to C6 alkylamino, (xv) 5- to 6-membered cyclic amino, (xvi) C1 to C6 alkyl-carbonyl, (xvii) carboxyl, (xviii) C1 to C6 alkoxycarbonyl optionally substituted with a halogen atom, (xix) C7 to C16 aralkyloxycarbonyl optionally substituted with a halogen atom, (xx) carbamoyl, (xxi) mono-C1 to C6 alkylcarbamoyl optionally substituted with 1 to 3 groups which are selected from the group consisting of a halogen atom, hydroxyl, carboxyl, C1 to C6 alkoxy, amino, and mono- or di-C1 to C6 alkylamino, (xxii) di-C1 to C6 alkylcarbamoyl optionally substituted with hydroxyl, (xxiii) 5- to 6-membered cyclic aminocarbonyl optionally substituted with C1 to C6 alkoxycarbonyl, (xxiv) C6 to C10 arylcarbamoyl, (xxv) C1 to C10 heteroarylcarbamoyl, (xxvi) C7 to C16 aralkylcarbamoyl, (xxvii) C1 to C10 heteroaryl-C1 to C6 alkylcarbamoyl, (xxviii) N C1 to C6 alkyl-N—C6 to C12 arylcarbamoyl, (xxix) C3 to C6 cycloalkylcarbamoyl, (xxx) sulfo, (xxxi) C1 to C6 alkylsulfonyl, (xxxii) C1 to C6 alkylsulfonylamino, (xxxiii) C6 to C12 arylsulfonylamino optionally substituted with C1 to C6 alkyl, (xxxiv) C1 to C10 heteroarylsulfonylamino, (xxxv) C1 to C6 alkoxycarbonylamino, (xxxvi) C1 to C6 alkylcarbonylamino, (xxxvii) mono- or di-C1 to C6 alkylaminocarbonylamino, (xxxviii) C6 to C12 aryl, (xxxix) C1 to C10 heteroaryl, (xl) C6 to C10 aryloxy, (xli) C1 to C10 heteroaryloxy, (xlii) C7 to C16 aralkyloxy, (xliii) C1 to C10 heteroaryl-C1 to C6 alkyloxy, (xliv) aminosulfonyl, (xlv) mono- or di-C1 to C6 alkylaminosulfonyl, (xlvi) C7 to C16 aralkyloxycarbamoyl, and (xlvii) C1 to C10 heteroaryl-C1 to C6 alkyloxycarbamoyl, or substituted with 1 to 9 deuterium atoms,
R1 is
(6) a hydrogen atom,
(7) C1 to C6 alkyl optionally substituted with 1 to 3 of (i) a halogen atom or (ii) C3 to C6 cycloalkyl or substituted with (iii) 1 to 13 deuterium atoms,
(8) C2 to C6 alkenyl optionally substituted with 1 to 3 of (i) a halogen atom or (ii) C3 to C6 cycloalkyl or substituted with (iii) 1 to 11 deuterium atoms,
(9) C2 to C6 alkynyl optionally substituted with 1 to 3 of (i) a halogen atom or (ii) C3 to C6 cycloalkyl, or substituted with (iii) 1 to 9 deuterium atoms, or
(10) C3 to C6 cycloalkyl optionally substituted with 1 to 3 of (i) a halogen atom or (ii) C3 to C6 cycloalkyl, or substituted with (iii) 1 to 11 deuterium atoms,
R2 is (1) OR⁹ or (2) NR¹⁰R¹¹
where, R⁹, R¹⁰, and R¹¹ independently indicate
(E) a hydrogen atom,
(F) C1 to C6 alkyl,
(G) C2 to C6 alkenyl,
(H) C3 to C6 alkynyl,
(I) C3 to C6 cycloalkyl
(J) C6 to C14 aromatic hydrocarbon group,
(K) a 5- to 8-membered aromatic heterocyclic group having 1 to 4 hetero atoms selected from the group consisting of a nitrogen atom, sulfur atom, and oxygen atom, in place of a carbon atom, or
(L) a bicyclic or tricyclic aromatic group formed by condensation of the above aromatic heterocyclic group and a C6 to C14 aromatic hydrocarbon ring,
where each of the groups of the above (B) to (E) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5 to 6-membered cyclic amino, (ix) carboxyl, (x) C1 to C6 alkoxycarbonyl, (xi) C1 to C6 alkylcarbonyl, (xii) carbamoyl, (xiii) mono-C1 to C6 alkylcarbamoyl, (xiv) di-C1 to C6 alkylcarbamoyl, (xv) C6 to C12 aryl, and (xvi) C1 to C10 heteroaryl, or substituted with 1 to 13 deuterium atoms,
further, each of the groups of the above (F) to (H) are optionally substituted with 1 to 5 groups which are selected from the group consisting of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms, or R¹ and R² may form 5- or 6-member heterocyclic ring together with the atoms they are bonded with, where the above 5- or 6-member heterocyclic ring is optionally substituted with 1 to 3 groups selected from the group consisting of
(I) a halogen atom,
(J) oxo,
(K) hydroxyl
(L) C1 to C6 alkyl,
(M) C2 to C6 alkenyl,
(N) C2 to C6 alkynyl,
(O) C3 to C6 cycloalkyl
(P) C6 to C14 aromatic hydrocarbon group,
(L) 5- to 8-membered aromatic heterocyclic group having 1 to 4 hetero atoms selected from the group consisting of a nitrogen atom, sulfur atom, and oxygen atom, in place of a carbon atom, and
(M) a bicyclic or tricyclic aromatic group formed by condensation of the above aromatic heterocyclic group and a C6 to C14 aromatic hydrocarbon ring, or substituted with
(N) 1 to 6 deuterium atoms,
where each of the groups of the above (D) to (G) is optionally substituted with 1 to 3 groups selected from the group consisting of (i) a halogen atom, (ii) C3 to C6 cycloalkyl, (iii) hydroxyl, (iv) C1 to C6 alkoxy optionally substituted with 1 to 3 halogen atoms, (v) amino, (vi) mono-C1 to C6 alkylamino, (vii) di-C1 to C6 alkylamino, (viii) 5- to 6-membered cyclic amino, (ix) carboxyl, (x) C1 to C6 alkoxycarbonyl, (xi) C1 to C6 alkyl-carbonyl, (xii) carbamoyl, (xiii) mono-C1 to C6 alkylcarbamoyl, (xiv) di-C1 to C6 alkylcarbamoyl, (xv) C6 to C12 aryl and (xvi) C1 to C10 heteroaryl, or substituted with 1 to 13 deuterium atoms, each of the groups of the above (H) to (J) is optionally substituted with 1 to 5 groups which are selected from the group consisting, of (i) a halogen atom, (ii) nitro, (iii) cyano, (iv) C1 to C6 alkyl optionally substituted with 1 to 3 halogen atoms, (v) C3 to C6 cycloalkyl optionally substituted with 1 to 3 halogen atoms, (vi) hydroxyl, (vii) C1 to C6 alkoxy optionally substituted with 1 to 3 halogen atoms, (viii) amino, (ix) mono-C1 to C6 alkylamino, (x) di-C1 to C6 alkylamino, (xi) 5- to 6-membered cyclic amino, (xii) C1 to C6 alkylcarbonyl, (xiii) carboxyl, (xiv) C1 to C6 alkoxycarbonyl, (xv) carbamoyl, (xvi) mono-C1 to C6 alkylcarbamoyl, (xvii) di-C1 to C6 alkylcarbamoyl, (xviii) C1 to C6 alkylsulfonyl, (xix) aminosulfonyl, and (xx) mono- or di-C1 to C6 alkylaminosulfonyl, or substituted with 1 to 9 deuterium atoms.

28. The method of claim 27, wherein said administration of said therapeutically effective amount of compound of Formula I or a pharmaceutically acceptable salt or solvate thereof ameliorates one or more of infiltration of at least one of mast cells or macrophages, bile acid accumulation, cholangiocyte proliferation, biliary senescence, fibrosis, collagen deposition in said subject suffering from hepatobiliary disease.

29. The method of claim 27, wherein said administration of said therapeutically effective amount of compound of Formula I or a pharmaceutically acceptable salt or solvate thereof ameliorates one or more of occult blood in stool, fecal incontinence, fever, fatigue, weight loss, mast cell and eosinophil counts, dysphagia, gastroesophageal reflux disease (GERD), bloating, early satiety, loss of appetite, jaundice, pruritis, hepatomegaly, cirrhosis, biliary obstruction, abdominal pain, nausea, diarrhea, spasms, Ulcerative colitis, Crohn's disease, Irritable bowel syndrome (IBS), Proctitis, Celiac disease, ileitis, duodenitis, diverticulitis, and inflammation in said subject suffering from hepatobiliary disease.

30. The method of claim 27, wherein the compound of formula (I) or a pharmaceutically acceptable salt or solvate thereof is a selective for chymase over cathepsin G.

31. The method of claim 27, wherein the hepatobiliary disease is selected from the group consisting of acute hepatitis, alcoholic hepatitis, alcoholic liver disease, extrahepatic cholestasis, hepatic fibrosis, liver abscess, liver cirrhosis, colitis, neonatal jaundice, obstructive jaundice, primary bile acid malabsorption, acute cholecystitis, autoimmune hepatitis, benign recurrent intrahepatic cholestasis, biliary obstruction, cholestatic liver disease, crigler-najjar syndrome, drug-induced hepatitis, gilbert's syndrome, intrahepatic cholestasis of pregnancy, liver rejection, non-alcoholic fatty liver disease, non-alcoholic steatohepatitis, polycystic liver disease, veno-occlusive disease, and Wilson's disease.

33. The method of claim 27, wherein said therapeutically effective amount of compound of Formula I or a pharmaceutically acceptable salt or solvate thereof is from about 20 mg to about 400 mg per unit dose.
